SEQUENCE LISTING (584 sequences)

SEQ ID NO: 1 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 2 - protein, 10 aa, Cytomegalovirus
KMQVIGDQYV

SEQ ID NO: 3 - protein, 10 aa, Cytomegalovirus
FTWPPWQAGI

SEQ ID NO: 4 - protein, 9 aa, Cytomegalovirus
AMAGASTSA

SEQ ID NO: 5 - protein, 10 aa, Cytomegalovirus
SDNEIHNPAV

SEQ ID NO: 6 - protein, 9 aa, Cytomegalovirus
KYQEFFWDA

SEQ ID NO: 7 - protein, 9 aa, Cytomegalovirus
RLLQTGIHV

SEQ ID NO: 8 - protein, 9 aa, Cytomegalovirus
VIGDQYVKV

SEQ ID NO: 9 - protein, 9 aa, Cytomegalovirus
YLESFCEDV

SEQ ID NO: 10 - protein, 10 aa, Cytomegalovirus
RPHERNGFTV

SEQ ID NO: 11 - protein, 12 aa, Cytomegalovirus
DDVWTSGSDSDE

SEQ ID NO: 12 - protein, 10 aa, Cytomegalovirus
TPRVTGGGAM

SEQ ID NO: 13 - protein, 10 aa, Cytomegalovirus
KMQVIGDQYV

SEQ ID NO: 14 - protein, 9 aa, Cytomegalovirus
GILGFVFTL

SEQ ID NO: 15 - protein, 9 aa, Cytomegalovirus
GLCTLVAML

SEQ ID NO: 16 - protein, 14 aa, Cytomegalovirus
YARFQSQTTLKQKT

SEQ ID NO: 17 - protein, 14 aa, Cytomegalovirus
YPKYVKQNTLKLAT

SEQ ID NO: 18 - protein, 20 aa, Cytomegalovirus
IDELKTNSSLLTSILTYHVV

SEQ ID NO: 19 - protein, 15 aa, Cytomegalovirus
TGSGAGIAQAAAGTV

SEQ ID NO: 20 - protein, 16 aa, Cytomegalovirus
GVSTANATVYMIDSVL

SEQ ID NO: 21 - protein, 16 aa, Cytomegalovirus
NFAGIEAAASAIQGNV

SEQ ID NO: 22 - protein, 17 aa, Cytomegalovirus
AETPGCVAYIGISFLDQ

SEQ ID NO: 23 - protein, 16 aa, Cytomegalovirus
VSDLKSSTAVIPGYPV

SEQ ID NO: 24 - protein, 16 aa, Cytomegalovirus
NFLLPDAQSIQAAAAG

SEQ ID NO: 25 - protein, 17 aa, Cytomegalovirus
YNINISLPSYYPDQKSL

SEQ ID NO: 26 - protein, 15 aa, Cytomegalovirus
MESRGRRCPEMISVL

SEQ ID NO: 27 - protein, 15 aa, Cytomegalovirus
ESRGRRCPEMISVLG

SEQ ID NO: 28 - protein, 15 aa, Cytomegalovirus
SRGRRCPEMISVLGP

SEQ ID NO: 29 - protein, 15 aa, Cytomegalovirus
RGRRCPEMISVLGPI

SEQ ID NO: 30 - protein, 15 aa, Cytomegalovirus
GRRCPEMISVLGPIS

SEQ ID NO: 31 - protein, 15 aa, Cytomegalovirus
RRCPEMISVLGPISG

SEQ ID NO: 32 - protein, 15 aa, Cytomegalovirus
RCPEMISVLGPISGH

SEQ ID NO: 33 - protein, 15 aa, Cytomegalovirus
CPEMISVLGPISGHV

SEQ ID NO: 34 - protein, 15 aa, Cytomegalovirus
PEMISVLGPISGHVL

SEQ ID NO: 35 - protein, 15 aa, Cytomegalovirus
EMISVLGPISGHVLK

SEQ ID NO: 36 - protein, 15 aa, Cytomegalovirus
MISVLGPISGHVLKA

SEQ ID NO: 37 - protein, 15 aa, Cytomegalovirus
ISVLGPISGHVLKAV

SEQ ID NO: 38 - protein, 15 aa, Cytomegalovirus
SVLGPISGHVLKAVF

SEQ ID NO: 39 - protein, 15 aa, Cytomegalovirus
VLGPISGHVLKAVFS

SEQ ID NO: 40 - protein, 15 aa, Cytomegalovirus
LGPISGHVLKAVFSR

SEQ ID NO: 41 - protein, 15 aa, Cytomegalovirus
GPISGHVLKAVFSRG

SEQ ID NO: 42 - protein, 15 aa, Cytomegalovirus
PISGHVLKAVFSRGD

SEQ ID NO: 43 - protein, 15 aa, Cytomegalovirus
ISGHVLKAVFSRGDT

SEQ ID NO: 44 - protein, 15 aa, Cytomegalovirus
SGHVLKAVFSRGDTP

SEQ ID NO: 45 - protein, 15 aa, Cytomegalovirus
GHVLKAVFSRGDTPV

SEQ ID NO: 46 - protein, 15 aa, Cytomegalovirus
HVLKAVFSRGDTPVL

SEQ ID NO: 47 - protein, 15 aa, Cytomegalovirus
VLKAVFSRGDTPVLP

SEQ ID NO: 48 - protein, 15 aa, Cytomegalovirus
LKAVFSRGDTPVLPH

SEQ ID NO: 49 - protein, 15 aa, Cytomegalovirus
KAVFSRGDTPVLPHE

SEQ ID NO: 50 - protein, 15 aa, Cytomegalovirus
AVFSRGDTPVLPHET

SEQ ID NO: 51 - protein, 15 aa, Cytomegalovirus
VFSRGDTPVLPHETR

SEQ ID NO: 52 - protein, 15 aa, Cytomegalovirus
FSRGDTPVLPHETRL

SEQ ID NO: 53 - protein, 15 aa, Cytomegalovirus
SRGDTPVLPHETRLL

SEQ ID NO: 54 - protein, 15 aa, Cytomegalovirus
RGDTPVLPHETRLLQ

SEQ ID NO: 55 - protein, 15 aa, Cytomegalovirus
GDTPVLPHETRLLQT

SEQ ID NO: 56 - protein, 15 aa, Cytomegalovirus
DTPVLPHETRLLQTG

SEQ ID NO: 57 - protein, 15 aa, Cytomegalovirus
TPVLPHETRLLQTGI

SEQ ID NO: 58 - protein, 15 aa, Cytomegalovirus
PVLPHETRLLQTGIH

SEQ ID NO: 59 - protein, 15 aa, Cytomegalovirus
VLPHETRLLQTGIHV

SEQ ID NO: 60 - protein, 15 aa, Cytomegalovirus
LPHETRLLQTGIHVR

SEQ ID NO: 61 - protein, 15 aa, Cytomegalovirus
PHETRLLQTGIHVRV

SEQ ID NO: 62 - protein, 15 aa, Cytomegalovirus
HETRLLQTGIHVRVS

SEQ ID NO: 63 - protein, 15 aa, Cytomegalovirus
ETRLLQTGIHVRVSQ

SEQ ID NO: 64 - protein, 15 aa, Cytomegalovirus
TRLLQTGIHVRVSQP

SEQ ID NO: 65 - protein, 15 aa, Cytomegalovirus
RLLQTGIHVRVSQPS

SEQ ID NO: 66 - protein, 15 aa, Cytomegalovirus
LLQTGIHVRVSQPSL

SEQ ID NO: 67 - protein, 15 aa, Cytomegalovirus
LQTGIHVRVSQPSLI

SEQ ID NO: 68 - protein, 15 aa, Cytomegalovirus
QTGIHVRVSQPSLIL

SEQ ID NO: 69 - protein, 15 aa, Cytomegalovirus
TGIHVRVSQPSLILV

SEQ ID NO: 70 - protein, 15 aa, Cytomegalovirus
GIHVRVSQPSLILVS

SEQ ID NO: 71 - protein, 15 aa, Cytomegalovirus
IHVRVSQPSLILVSQ

SEQ ID NO: 72 - protein, 15 aa, Cytomegalovirus
HVRVSQPSLILVSQY

SEQ ID NO: 73 - protein, 15 aa, Cytomegalovirus
VRVSQPSLILVSQYT

SEQ ID NO: 74 - protein, 15 aa, Cytomegalovirus
RVSQPSLILVSQYTP

SEQ ID NO: 75 - protein, 15 aa, Cytomegalovirus
VSQPSLILVSQYTPD

SEQ ID NO: 76 - protein, 15 aa, Cytomegalovirus
SQPSLILVSQYTPDS

SEQ ID NO: 77 - protein, 15 aa, Cytomegalovirus
QPSLILVSQYTPDST

SEQ ID NO: 78 - protein, 15 aa, Cytomegalovirus
PSLILVSQYTPDSTP

SEQ ID NO: 79 - protein, 15 aa, Cytomegalovirus
SLILVSQYTPDSTPC

SEQ ID NO: 80 - protein, 15 aa, Cytomegalovirus
LILVSQYTPDSTPCH

SEQ ID NO: 81 - protein, 15 aa, Cytomegalovirus
ILVSQYTPDSTPCHR

SEQ ID NO: 82 - protein, 15 aa, Cytomegalovirus
LVSQYTPDSTPCHRG

SEQ ID NO: 83 - protein, 15 aa, Cytomegalovirus
VSQYTPDSTPCHRGD

SEQ ID NO: 84 - protein, 15 aa, Cytomegalovirus
SQYTPDSTPCHRGDN

SEQ ID NO: 85 - protein, 15 aa, Cytomegalovirus
QYTPDSTPCHRGDNQ

SEQ ID NO: 86 - protein, 15 aa, Cytomegalovirus
YTPDSTPCHRGDNQL

SEQ ID NO: 87 - protein, 15 aa, Cytomegalovirus
TPDSTPCHRGDNQLQ

SEQ ID NO: 88 - protein, 15 aa, Cytomegalovirus
PDSTPCHRGDNQLQV

SEQ ID NO: 89 - protein, 15 aa, Cytomegalovirus
DSTPCHRGDNQLQVQ

SEQ ID NO: 90 - protein, 15 aa, Cytomegalovirus
STPCHRGDNQLQVQH

SEQ ID NO: 91 - protein, 15 aa, Cytomegalovirus
TPCHRGDNQLQVQHT

SEQ ID NO: 92 - protein, 15 aa, Cytomegalovirus
PCHRGDNQLQVQHTY

SEQ ID NO: 93 - protein, 15 aa, Cytomegalovirus
CHRGDNQLQVQHTYF

SEQ ID NO: 94 - protein, 15 aa, Cytomegalovirus
HRGDNQLQVQHTYFT

SEQ ID NO: 95 - protein, 15 aa, Cytomegalovirus
RGDNQLQVQHTYFTG

SEQ ID NO: 96 - protein, 15 aa, Cytomegalovirus
GDNQLQVQHTYFTGS

SEQ ID NO: 97 - protein, 15 aa, Cytomegalovirus
DNQLQVQHTYFTGSE

SEQ ID NO: 98 - protein, 15 aa, Cytomegalovirus
NQLQVQHTYFTGSEV

SEQ ID NO: 99 - protein, 15 aa, Cytomegalovirus
QLQVQHTYFTGSEVE

SEQ ID NO: 100 - protein, 15 aa, Cytomegalovirus
LQVQHTYFTGSEVEN

SEQ ID NO: 101 - protein, 15 aa, Cytomegalovirus
QVQHTYFTGSEVENV

SEQ ID NO: 102 - protein, 15 aa, Cytomegalovirus
VQHTYFTGSEVENVS

SEQ ID NO: 103 - protein, 15 aa, Cytomegalovirus
QHTYFTGSEVENVSV

SEQ ID NO: 104 - protein, 15 aa, Cytomegalovirus
HTYFTGSEVENVSVN

SEQ ID NO: 105 - protein, 15 aa, Cytomegalovirus
TYFTGSEVENVSVNV

SEQ ID NO: 106 - protein, 15 aa, Cytomegalovirus
YFTGSEVENVSVNVH

SEQ ID NO: 107 - protein, 15 aa, Cytomegalovirus
FTGSEVENVSVNVHN

SEQ ID NO: 108 - protein, 15 aa, Cytomegalovirus
TGSEVENVSVNVHNP

SEQ ID NO: 109 - protein, 15 aa, Cytomegalovirus
GSEVENVSVNVHNPT

SEQ ID NO: 110 - protein, 15 aa, Cytomegalovirus
SEVENVSVNVHNPTG

SEQ ID NO: 111 - protein, 15 aa, Cytomegalovirus
EVENVSVNVHNPTGR

SEQ ID NO: 112 - protein, 15 aa, Cytomegalovirus
VENVSVNVHNPTGRS

SEQ ID NO: 113 - protein, 15 aa, Cytomegalovirus
ENVSVNVHNPTGRSI

SEQ ID NO: 114 - protein, 15 aa, Cytomegalovirus
NVSVNVHNPTGRSIC

SEQ ID NO: 115 - protein, 15 aa, Cytomegalovirus
VSVNVHNPTGRSICP

SEQ ID NO: 116 - protein, 15 aa, Cytomegalovirus
SVNVHNPTGRSICPS

SEQ ID NO: 117 - protein, 15 aa, Cytomegalovirus
VNVHNPTGRSICPSQ

SEQ ID NO: 118 - protein, 15 aa, Cytomegalovirus
NVHNPTGRSICPSQE

SEQ ID NO: 119 - protein, 15 aa, Cytomegalovirus
VHNPTGRSICPSQEP

SEQ ID NO: 120 - protein, 15 aa, Cytomegalovirus
HNPTGRSICPSQEPM

SEQ ID NO: 121 - protein, 15 aa, Cytomegalovirus
NPTGRSICPSQEPMS

SEQ ID NO: 122 - protein, 15 aa, Cytomegalovirus
PTGRSICPSQEPMSI

SEQ ID NO: 123 - protein, 15 aa, Cytomegalovirus
TGRSICPSQEPMSIY

SEQ ID NO: 124 - protein, 15 aa, Cytomegalovirus
GRSICPSQEPMSIYV

SEQ ID NO: 125 - protein, 15 aa, Cytomegalovirus
RSICPSQEPMSIYVY

SEQ ID NO: 126 - protein, 15 aa, Cytomegalovirus
SICPSQEPMSIYVYA

SEQ ID NO: 127 - protein, 15 aa, Cytomegalovirus
ICPSQEPMSIYVYAL

SEQ ID NO: 128 - protein, 15 aa, Cytomegalovirus
CPSQEPMSIYVYALP

SEQ ID NO: 129 - protein, 15 aa, Cytomegalovirus
PSQEPMSIYVYALPL

SEQ ID NO: 130 - protein, 15 aa, Cytomegalovirus
SQEPMSIYVYALPLK

SEQ ID NO: 131 - protein, 15 aa, Cytomegalovirus
QEPMSIYVYALPLKM

SEQ ID NO: 132 - protein, 15 aa, Cytomegalovirus
EPMSIYVYALPLKML

SEQ ID NO: 133 - protein, 15 aa, Cytomegalovirus
PMSIYVYALPLKMLN

SEQ ID NO: 134 - protein, 15 aa, Cytomegalovirus
MSIYVYALPLKMLNI

SEQ ID NO: 135 - protein, 15 aa, Cytomegalovirus
SIYVYALPLKMLNIP

SEQ ID NO: 136 - protein, 15 aa, Cytomegalovirus
IYVYALPLKMLNIPS

SEQ ID NO: 137 - protein, 15 aa, Cytomegalovirus
YVYALPLKMLNIPSI

SEQ ID NO: 138 - protein, 15 aa, Cytomegalovirus
VYALPLKMLNIPSIN

SEQ ID NO: 139 - protein, 15 aa, Cytomegalovirus
YALPLKMLNIPSINV

SEQ ID NO: 140 - protein, 15 aa, Cytomegalovirus
ALPLKMLNIPSINVH

SEQ ID NO: 141 - protein, 15 aa, Cytomegalovirus
LPLKMLNIPSINVHH

SEQ ID NO: 142 - protein, 15 aa, Cytomegalovirus
PLKMLNIPSINVHHY

SEQ ID NO: 143 - protein, 15 aa, Cytomegalovirus
LKMLNIPSINVHHYP

SEQ ID NO: 144 - protein, 15 aa, Cytomegalovirus
KMLNIPSINVHHYPS

SEQ ID NO: 145 - protein, 15 aa, Cytomegalovirus
MLNIPSINVHHYPSA

SEQ ID NO: 146 - protein, 15 aa, Cytomegalovirus
LNIPSINVHHYPSAA

SEQ ID NO: 147 - protein, 15 aa, Cytomegalovirus
NIPSINVHHYPSAAE

SEQ ID NO: 148 - protein, 15 aa, Cytomegalovirus
IPSINVHHYPSAAER

SEQ ID NO: 149 - protein, 15 aa, Cytomegalovirus
PSINVHHYPSAAERK

SEQ ID NO: 150 - protein, 15 aa, Cytomegalovirus
SINVHHYPSAAERKH

SEQ ID NO: 151 - protein, 15 aa, Cytomegalovirus
INVHHYPSAAERKHR

SEQ ID NO: 152 - protein, 15 aa, Cytomegalovirus
NVHHYPSAAERKHRH

SEQ ID NO: 153 - protein, 15 aa, Cytomegalovirus
VHHYPSAAERKHRHL

SEQ ID NO: 154 - protein, 15 aa, Cytomegalovirus
HHYPSAAERKHRHLP

SEQ ID NO: 155 - protein, 15 aa, Cytomegalovirus
HYPSAAERKHRHLPV

SEQ ID NO: 156 - protein, 15 aa, Cytomegalovirus
YPSAAERKHRHLPVA

SEQ ID NO: 157 - protein, 15 aa, Cytomegalovirus
PSAAERKHRHLPVAD

SEQ ID NO: 158 - protein, 15 aa, Cytomegalovirus
SAAERKHRHLPVADA

SEQ ID NO: 159 - protein, 15 aa, Cytomegalovirus
AAERKHRHLPVADAV

SEQ ID NO: 160 - protein, 15 aa, Cytomegalovirus
AERKHRHLPVADAVI

SEQ ID NO: 161 - protein, 15 aa, Cytomegalovirus
ERKHRHLPVADAVIH

SEQ ID NO: 162 - protein, 15 aa, Cytomegalovirus
RKHRHLPVADAVIHA

SEQ ID NO: 163 - protein, 15 aa, Cytomegalovirus
KHRHLPVADAVIHAS

SEQ ID NO: 164 - protein, 15 aa, Cytomegalovirus
HRHLPVADAVIHASG

SEQ ID NO: 165 - protein, 15 aa, Cytomegalovirus
RHLPVADAVIHASGK

SEQ ID NO: 166 - protein, 15 aa, Cytomegalovirus
HLPVADAVIHASGKQ

SEQ ID NO: 167 - protein, 15 aa, Cytomegalovirus
LPVADAVIHASGKQM

SEQ ID NO: 168 - protein, 15 aa, Cytomegalovirus
PVADAVIHASGKQMW

SEQ ID NO: 169 - protein, 15 aa, Cytomegalovirus
VADAVIHASGKQMWQ

SEQ ID NO: 170 - protein, 15 aa, Cytomegalovirus
ADAVIHASGKQMWQA

SEQ ID NO: 171 - protein, 15 aa, Cytomegalovirus
DAVIHASGKQMWQAR

SEQ ID NO: 172 - protein, 15 aa, Cytomegalovirus
AVIHASGKQMWQARL

SEQ ID NO: 173 - protein, 15 aa, Cytomegalovirus
VIHASGKQMWQARLT

SEQ ID NO: 174 - protein, 15 aa, Cytomegalovirus
IHASGKQMWQARLTV

SEQ ID NO: 175 - protein, 15 aa, Cytomegalovirus
HASGKQMWQARLTVS

SEQ ID NO: 176 - protein, 15 aa, Cytomegalovirus
ASGKQMWQARLTVSG

SEQ ID NO: 177 - protein, 15 aa, Cytomegalovirus
SGKQMWQARLTVSGL

SEQ ID NO: 178 - protein, 15 aa, Cytomegalovirus
GKQMWQARLTVSGLA

SEQ ID NO: 179 - protein, 15 aa, Cytomegalovirus
KQMWQARLTVSGLAW

SEQ ID NO: 180 - protein, 15 aa, Cytomegalovirus
QMWQARLTVSGLAWT

SEQ ID NO: 181 - protein, 15 aa, Cytomegalovirus
MWQARLTVSGLAWTR

SEQ ID NO: 182 - protein, 15 aa, Cytomegalovirus
WQARLTVSGLAWTRQ

SEQ ID NO: 183 - protein, 15 aa, Cytomegalovirus
QARLTVSGLAWTRQQ

SEQ ID NO: 184 - protein, 15 aa, Cytomegalovirus
ARLTVSGLAWTRQQN

SEQ ID NO: 185 - protein, 15 aa, Cytomegalovirus
RLTVSGLAWTRQQNQ

SEQ ID NO: 186 - protein, 15 aa, Cytomegalovirus
LTVSGLAWTRQQNQW

SEQ ID NO: 187 - protein, 15 aa, Cytomegalovirus
TVSGLAWTRQQNQWK

SEQ ID NO: 188 - protein, 15 aa, Cytomegalovirus
VSGLAWTRQQNQWKE

SEQ ID NO: 189 - protein, 15 aa, Cytomegalovirus
SGLAWTRQQNQWKEP

SEQ ID NO: 190 - protein, 15 aa, Cytomegalovirus
GLAWTRQQNQWKEPD

SEQ ID NO: 191 - protein, 15 aa, Cytomegalovirus
LAWTRQQNQWKEPDV

SEQ ID NO: 192 - protein, 15 aa, Cytomegalovirus
AWTRQQNQWKEPDVY

SEQ ID NO: 193 - protein, 15 aa, Cytomegalovirus
WTRQQNQWKEPDVYY

SEQ ID NO: 194 - protein, 15 aa, Cytomegalovirus
TRQQNQWKEPDVYYT

SEQ ID NO: 195 - protein, 15 aa, Cytomegalovirus
RQQNQWKEPDVYYTS

SEQ ID NO: 196 - protein, 15 aa, Cytomegalovirus
QQNQWKEPDVYYTSA

SEQ ID NO: 197 - protein, 15 aa, Cytomegalovirus
QNQWKEPDVYYTSAF

SEQ ID NO: 198 - protein, 15 aa, Cytomegalovirus
NQWKEPDVYYTSAFV

SEQ ID NO: 199 - protein, 15 aa, Cytomegalovirus
QWKEPDVYYTSAFVF

SEQ ID NO: 200 - protein, 15 aa, Cytomegalovirus
WKEPDVYYTSAFVFP

SEQ ID NO: 201 - protein, 15 aa, Cytomegalovirus
KEPDVYYTSAFVFPT

SEQ ID NO: 202 - protein, 15 aa, Cytomegalovirus
EPDVYYTSAFVFPTK

SEQ ID NO: 203 - protein, 15 aa, Cytomegalovirus
PDVYYTSAFVFPTKD

SEQ ID NO: 204 - protein, 15 aa, Cytomegalovirus
DVYYTSAFVFPTKDV

SEQ ID NO: 205 - protein, 15 aa, Cytomegalovirus
VYYTSAFVFPTKDVA

SEQ ID NO: 206 - protein, 15 aa, Cytomegalovirus
YYTSAFVFPTKDVAL

SEQ ID NO: 207 - protein, 15 aa, Cytomegalovirus
YTSAFVFPTKDVALR

SEQ ID NO: 208 - protein, 15 aa, Cytomegalovirus
TSAFVFPTKDVALRH

SEQ ID NO: 209 - protein, 15 aa, Cytomegalovirus
SAFVFPTKDVALRHV

SEQ ID NO: 210 - protein, 15 aa, Cytomegalovirus
AFVFPTKDVALRHVV

SEQ ID NO: 211 - protein, 15 aa, Cytomegalovirus
FVFPTKDVALRHVVC

SEQ ID NO: 212 - protein, 15 aa, Cytomegalovirus
VFPTKDVALRHVVCA

SEQ ID NO: 213 - protein, 15 aa, Cytomegalovirus
FPTKDVALRHVVCAH

SEQ ID NO: 214 - protein, 15 aa, Cytomegalovirus
PTKDVALRHVVCAHE

SEQ ID NO: 215 - protein, 15 aa, Cytomegalovirus
TKDVALRHVVCAHEL

SEQ ID NO: 216 - protein, 15 aa, Cytomegalovirus
KDVALRHVVCAHELV

SEQ ID NO: 217 - protein, 15 aa, Cytomegalovirus
DVALRHVVCAHELVC

SEQ ID NO: 218 - protein, 15 aa, Cytomegalovirus
VALRHVVCAHELVCS

SEQ ID NO: 219 - protein, 15 aa, Cytomegalovirus
ALRHVVCAHELVCSM

SEQ ID NO: 220 - protein, 15 aa, Cytomegalovirus
LRHVVCAHELVCSME

SEQ ID NO: 221 - protein, 15 aa, Cytomegalovirus
RHVVCAHELVCSMEN

SEQ ID NO: 222 - protein, 15 aa, Cytomegalovirus
HVVCAHELVCSMENT

SEQ ID NO: 223 - protein, 15 aa, Cytomegalovirus
VVCAHELVCSMENTR

SEQ ID NO: 224 - protein, 15 aa, Cytomegalovirus
VCAHELVCSMENTRA

SEQ ID NO: 225 - protein, 15 aa, Cytomegalovirus
CAHELVCSMENTRAT

SEQ ID NO: 226 - protein, 15 aa, Cytomegalovirus
AHELVCSMENTRATK

SEQ ID NO: 227 - protein, 15 aa, Cytomegalovirus
HELVCSMENTRATKM

SEQ ID NO: 228 - protein, 15 aa, Cytomegalovirus
ELVCSMENTRATKMQ

SEQ ID NO: 229 - protein, 15 aa, Cytomegalovirus
LVCSMENTRATKMQV

SEQ ID NO: 230 - protein, 15 aa, Cytomegalovirus
VCSMENTRATKMQVI

SEQ ID NO: 231 - protein, 15 aa, Cytomegalovirus
CSMENTRATKMQVIG

SEQ ID NO: 232 - protein, 15 aa, Cytomegalovirus
SMENTRATKMQVIGD

SEQ ID NO: 233 - protein, 15 aa, Cytomegalovirus
MENTRATKMQVIGDQ

SEQ ID NO: 234 - protein, 15 aa, Cytomegalovirus
ENTRATKMQVIGDQY

SEQ ID NO: 235 - protein, 15 aa, Cytomegalovirus
NTRATKMQVIGDQYV

SEQ ID NO: 236 - protein, 15 aa, Cytomegalovirus
TRATKMQVIGDQYVK

SEQ ID NO: 237 - protein, 15 aa, Cytomegalovirus
RATKMQVIGDQYVKV

SEQ ID NO: 238 - protein, 15 aa, Cytomegalovirus
ATKMQVIGDQYVKVY

SEQ ID NO: 239 - protein, 15 aa, Cytomegalovirus
TKMQVIGDQYVKVYL

SEQ ID NO: 240 - protein, 15 aa, Cytomegalovirus
KMQVIGDQYVKVYLE

SEQ ID NO: 241 - protein, 15 aa, Cytomegalovirus
MQVIGDQYVKVYLES

SEQ ID NO: 242 - protein, 15 aa, Cytomegalovirus
QVIGDQYVKVYLESF

SEQ ID NO: 243 - protein, 15 aa, Cytomegalovirus
VIGDQYVKVYLESFC

SEQ ID NO: 244 - protein, 15 aa, Cytomegalovirus
IGDQYVKVYLESFCE

SEQ ID NO: 245 - protein, 15 aa, Cytomegalovirus
GDQYVKVYLESFCED

SEQ ID NO: 246 - protein, 15 aa, Cytomegalovirus
DQYVKVYLESFCEDV

SEQ ID NO: 247 - protein, 15 aa, Cytomegalovirus
QYVKVYLESFCEDVP

SEQ ID NO: 248 - protein, 15 aa, Cytomegalovirus
YVKVYLESFCEDVPS

SEQ ID NO: 249 - protein, 15 aa, Cytomegalovirus
VKVYLESFCEDVPSG

SEQ ID NO: 250 - protein, 15 aa, Cytomegalovirus
KVYLESFCEDVPSGK

SEQ ID NO: 251 - protein, 15 aa, Cytomegalovirus
VYLESFCEDVPSGKL

SEQ ID NO: 252 - protein, 15 aa, Cytomegalovirus
YLESFCEDVPSGKLF

SEQ ID NO: 253 - protein, 15 aa, Cytomegalovirus
LESFCEDVPSGKLFM

SEQ ID NO: 254 - protein, 15 aa, Cytomegalovirus
ESFCEDVPSGKLFMH

SEQ ID NO: 255 - protein, 15 aa, Cytomegalovirus
SFCEDVPSGKLFMHV

SEQ ID NO: 256 - protein, 15 aa, Cytomegalovirus
FCEDVPSGKLFMHVT

SEQ ID NO: 257 - protein, 15 aa, Cytomegalovirus
CEDVPSGKLFMHVTL

SEQ ID NO: 258 - protein, 15 aa, Cytomegalovirus
EDVPSGKLFMHVTLG

SEQ ID NO: 259 - protein, 15 aa, Cytomegalovirus
DVPSGKLFMHVTLGS

SEQ ID NO: 260 - protein, 15 aa, Cytomegalovirus
VPSGKLFMHVTLGSD

SEQ ID NO: 261 - protein, 15 aa, Cytomegalovirus
PSGKLFMHVTLGSDV

SEQ ID NO: 262 - protein, 15 aa, Cytomegalovirus
SGKLFMHVTLGSDVE

SEQ ID NO: 263 - protein, 15 aa, Cytomegalovirus
GKLFMHVTLGSDVEE

SEQ ID NO: 264 - protein, 15 aa, Cytomegalovirus
KLFMHVTLGSDVEED

SEQ ID NO: 265 - protein, 15 aa, Cytomegalovirus
LFMHVTLGSDVEEDL

SEQ ID NO: 266 - protein, 15 aa, Cytomegalovirus
FMHVTLGSDVEEDLT

SEQ ID NO: 267 - protein, 15 aa, Cytomegalovirus
MHVTLGSDVEEDLTM

SEQ ID NO: 268 - protein, 15 aa, Cytomegalovirus
HVTLGSDVEEDLTMT

SEQ ID NO: 269 - protein, 15 aa, Cytomegalovirus
VTLGSDVEEDLTMTR

SEQ ID NO: 270 - protein, 15 aa, Cytomegalovirus
TLGSDVEEDLTMTRN

SEQ ID NO: 271 - protein, 15 aa, Cytomegalovirus
LGSDVEEDLTMTRNP

SEQ ID NO: 272 - protein, 15 aa, Cytomegalovirus
GSDVEEDLTMTRNPQ

SEQ ID NO: 273 - protein, 15 aa, Cytomegalovirus
SDVEEDLTMTRNPQP

SEQ ID NO: 274 - protein, 15 aa, Cytomegalovirus
DVEEDLTMTRNPQPF

SEQ ID NO: 275 - protein, 15 aa, Cytomegalovirus
VEEDLTMTRNPQPFM

SEQ ID NO: 276 - protein, 15 aa, Cytomegalovirus
EEDLTMTRNPQPFMR

SEQ ID NO: 277 - protein, 15 aa, Cytomegalovirus
EDLTMTRNPQPFMRP

SEQ ID NO: 278 - protein, 15 aa, Cytomegalovirus
DLTMTRNPQPFMRPH

SEQ ID NO: 279 - protein, 15 aa, Cytomegalovirus
LTMTRNPQPFMRPHE

SEQ ID NO: 280 - protein, 15 aa, Cytomegalovirus
TMTRNPQPFMRPHER

SEQ ID NO: 281 - protein, 15 aa, Cytomegalovirus
MTRNPQPFMRPHERN

SEQ ID NO: 282 - protein, 15 aa, Cytomegalovirus
TRNPQPFMRPHERNG

SEQ ID NO: 283 - protein, 15 aa, Cytomegalovirus
RNPQPFMRPHERNGF

SEQ ID NO: 284 - protein, 15 aa, Cytomegalovirus
NPQPFMRPHERNGFT

SEQ ID NO: 285 - protein, 15 aa, Cytomegalovirus
PQPFMRPHERNGFTV

SEQ ID NO: 286 - protein, 15 aa, Cytomegalovirus
QPFMRPHERNGFTVL

SEQ ID NO: 287 - protein, 15 aa, Cytomegalovirus
PFMRPHERNGFTVLC

SEQ ID NO: 288 - protein, 15 aa, Cytomegalovirus
FMRPHERNGFTVLCP

SEQ ID NO: 289 - protein, 15 aa, Cytomegalovirus
MRPHERNGFTVLCPK

SEQ ID NO: 290 - protein, 15 aa, Cytomegalovirus
RPHERNGFTVLCPKN

SEQ ID NO: 291 - protein, 15 aa, Cytomegalovirus
PHERNGFTVLCPKNM

SEQ ID NO: 292 - protein, 15 aa, Cytomegalovirus
HERNGFTVLCPKNMI

SEQ ID NO: 293 - protein, 15 aa, Cytomegalovirus
ERNGFTVLCPKNMII

SEQ ID NO: 294 - protein, 15 aa, Cytomegalovirus
RNGFTVLCPKNMIIK

SEQ ID NO: 295 - protein, 15 aa, Cytomegalovirus
NGFTVLCPKNMIIKP

SEQ ID NO: 296 - protein, 15 aa, Cytomegalovirus
GFTVLCPKNMIIKPG

SEQ ID NO: 297 - protein, 15 aa, Cytomegalovirus
FTVLCPKNMIIKPGK

SEQ ID NO: 298 - protein, 15 aa, Cytomegalovirus
TVLCPKNMIIKPGKI

SEQ ID NO: 299 - protein, 15 aa, Cytomegalovirus
VLCPKNMIIKPGKIS

SEQ ID NO: 300 - protein, 15 aa, Cytomegalovirus
LCPKNMIIKPGKISH

SEQ ID NO: 301 - protein, 15 aa, Cytomegalovirus
CPKNMIIKPGKISHI

SEQ ID NO: 302 - protein, 15 aa, Cytomegalovirus
PKNMIIKPGKISHIM

SEQ ID NO: 303 - protein, 15 aa, Cytomegalovirus
KNMIIKPGKISHIML

SEQ ID NO: 304 - protein, 15 aa, Cytomegalovirus
NMIIKPGKISHIMLD

SEQ ID NO: 305 - protein, 15 aa, Cytomegalovirus
MIIKPGKISHIMLDV

SEQ ID NO: 306 - protein, 15 aa, Cytomegalovirus
IIKPGKISHIMLDVA

SEQ ID NO: 307 - protein, 15 aa, Cytomegalovirus
IKPGKISHIMLDVAF

SEQ ID NO: 308 - protein, 15 aa, Cytomegalovirus
KPGKISHIMLDVAFT

SEQ ID NO: 309 - protein, 15 aa, Cytomegalovirus
PGKISHIMLDVAFTS

SEQ ID NO: 310 - protein, 15 aa, Cytomegalovirus
GKISHIMLDVAFTSH

SEQ ID NO: 311 - protein, 15 aa, Cytomegalovirus
KISHIMLDVAFTSHE

SEQ ID NO: 312 - protein, 15 aa, Cytomegalovirus
ISHIMLDVAFTSHEH

SEQ ID NO: 313 - protein, 15 aa, Cytomegalovirus
SHIMLDVAFTSHEHF

SEQ ID NO: 314 - protein, 15 aa, Cytomegalovirus
HIMLDVAFTSHEHFG

SEQ ID NO: 315 - protein, 15 aa, Cytomegalovirus
IMLDVAFTSHEHFGL

SEQ ID NO: 316 - protein, 15 aa, Cytomegalovirus
MLDVAFTSHEHFGLL

SEQ ID NO: 317 - protein, 15 aa, Cytomegalovirus
LDVAFTSHEHFGLLC

SEQ ID NO: 318 - protein, 15 aa, Cytomegalovirus
DVAFTSHEHFGLLCP

SEQ ID NO: 319 - protein, 15 aa, Cytomegalovirus
VAFTSHEHFGLLCPK

SEQ ID NO: 320 - protein, 15 aa, Cytomegalovirus
AFTSHEHFGLLCPKS

SEQ ID NO: 321 - protein, 15 aa, Cytomegalovirus
FTSHEHFGLLCPKSI

SEQ ID NO: 322 - protein, 15 aa, Cytomegalovirus
TSHEHFGLLCPKSIP

SEQ ID NO: 323 - protein, 15 aa, Cytomegalovirus
SHEHFGLLCPKSIPG

SEQ ID NO: 324 - protein, 15 aa, Cytomegalovirus
HEHFGLLCPKSIPGL

SEQ ID NO: 325 - protein, 15 aa, Cytomegalovirus
EHFGLLCPKSIPGLS

SEQ ID NO: 326 - protein, 15 aa, Cytomegalovirus
HFGLLCPKSIPGLSI

SEQ ID NO: 327 - protein, 15 aa, Cytomegalovirus
FGLLCPKSIPGLSIS

SEQ ID NO: 328 - protein, 15 aa, Cytomegalovirus
GLLCPKSIPGLSISG

SEQ ID NO: 329 - protein, 15 aa, Cytomegalovirus
LLCPKSIPGLSISGN

SEQ ID NO: 330 - protein, 15 aa, Cytomegalovirus
LCPKSIPGLSISGNL

SEQ ID NO: 331 - protein, 15 aa, Cytomegalovirus
CPKSIPGLSISGNLL

SEQ ID NO: 332 - protein, 15 aa, Cytomegalovirus
PKSIPGLSISGNLLM

SEQ ID NO: 333 - protein, 15 aa, Cytomegalovirus
KSIPGLSISGNLLMN

SEQ ID NO: 334 - protein, 15 aa, Cytomegalovirus
SIPGLSISGNLLMNG

SEQ ID NO: 335 - protein, 15 aa, Cytomegalovirus
IPGLSISGNLLMNGQ

SEQ ID NO: 336 - protein, 15 aa, Cytomegalovirus
PGLSISGNLLMNGQQ

SEQ ID NO: 337 - protein, 15 aa, Cytomegalovirus
GLSISGNLLMNGQQI

SEQ ID NO: 338 - protein, 15 aa, Cytomegalovirus
LSISGNLLMNGQQIF

SEQ ID NO: 339 - protein, 15 aa, Cytomegalovirus
SISGNLLMNGQQIFL

SEQ ID NO: 340 - protein, 15 aa, Cytomegalovirus
ISGNLLMNGQQIFLE

SEQ ID NO: 341 - protein, 15 aa, Cytomegalovirus
SGNLLMNGQQIFLEV

SEQ ID NO: 342 - protein, 15 aa, Cytomegalovirus
GNLLMNGQQIFLEVQ

SEQ ID NO: 343 - protein, 15 aa, Cytomegalovirus
NLLMNGQQIFLEVQA

SEQ ID NO: 344 - protein, 15 aa, Cytomegalovirus
LLMNGQQIFLEVQAI

SEQ ID NO: 345 - protein, 15 aa, Cytomegalovirus
LMNGQQIFLEVQAIR

SEQ ID NO: 346 - protein, 15 aa, Cytomegalovirus
MNGQQIFLEVQAIRE

SEQ ID NO: 347 - protein, 15 aa, Cytomegalovirus
NGQQIFLEVQAIRET

SEQ ID NO: 348 - protein, 15 aa, Cytomegalovirus
GQQIFLEVQAIRETV

SEQ ID NO: 349 - protein, 15 aa, Cytomegalovirus
QQIFLEVQAIRETVE

SEQ ID NO: 350 - protein, 15 aa, Cytomegalovirus
QIFLEVQAIRETVEL

SEQ ID NO: 351 - protein, 15 aa, Cytomegalovirus
IFLEVQAIRETVELR

SEQ ID NO: 352 - protein, 15 aa, Cytomegalovirus
FLEVQAIRETVELRQ

SEQ ID NO: 353 - protein, 15 aa, Cytomegalovirus
LEVQAIRETVELRQY

SEQ ID NO: 354 - protein, 15 aa, Cytomegalovirus
EVQAIRETVELRQYD

SEQ ID NO: 355 - protein, 15 aa, Cytomegalovirus
VQAIRETVELRQYDP

SEQ ID NO: 356 - protein, 15 aa, Cytomegalovirus
QAIRETVELRQYDPV

SEQ ID NO: 357 - protein, 15 aa, Cytomegalovirus
AIRETVELRQYDPVA

SEQ ID NO: 358 - protein, 15 aa, Cytomegalovirus
IRETVELRQYDPVAA

SEQ ID NO: 359 - protein, 15 aa, Cytomegalovirus
RETVELRQYDPVAAL

SEQ ID NO: 360 - protein, 15 aa, Cytomegalovirus
ETVELRQYDPVAALF

SEQ ID NO: 361 - protein, 15 aa, Cytomegalovirus
TVELRQYDPVAALFF

SEQ ID NO: 362 - protein, 15 aa, Cytomegalovirus
VELRQYDPVAALFFF

SEQ ID NO: 363 - protein, 15 aa, Cytomegalovirus
ELRQYDPVAALFFFD

SEQ ID NO: 364 - protein, 15 aa, Cytomegalovirus
LRQYDPVAALFFFDI

SEQ ID NO: 365 - protein, 15 aa, Cytomegalovirus
RQYDPVAALFFFDID

SEQ ID NO: 366 - protein, 15 aa, Cytomegalovirus
QYDPVAALFFFDIDL

SEQ ID NO: 367 - protein, 15 aa, Cytomegalovirus
YDPVAALFFFDIDLL

SEQ ID NO: 368 - protein, 15 aa, Cytomegalovirus
DPVAALFFFDIDLLL

SEQ ID NO: 369 - protein, 15 aa, Cytomegalovirus
PVAALFFFDIDLLLQ

SEQ ID NO: 370 - protein, 15 aa, Cytomegalovirus
VAALFFFDIDLLLQR

SEQ ID NO: 371 - protein, 15 aa, Cytomegalovirus
AALFFFDIDLLLQRG

SEQ ID NO: 372 - protein, 15 aa, Cytomegalovirus
ALFFFDIDLLLQRGP

SEQ ID NO: 373 - protein, 15 aa, Cytomegalovirus
LFFFDIDLLLQRGPQ

SEQ ID NO: 374 - protein, 15 aa, Cytomegalovirus
FFFDIDLLLQRGPQY

SEQ ID NO: 375 - protein, 15 aa, Cytomegalovirus
FFDIDLLLQRGPQYS

SEQ ID NO: 376 - protein, 15 aa, Cytomegalovirus
FDIDLLLQRGPQYSE

SEQ ID NO: 377 - protein, 15 aa, Cytomegalovirus
DIDLLLQRGPQYSEH

SEQ ID NO: 378 - protein, 15 aa, Cytomegalovirus
IDLLLQRGPQYSEHP

SEQ ID NO: 379 - protein, 15 aa, Cytomegalovirus
DLLLQRGPQYSEHPT

SEQ ID NO: 380 - protein, 15 aa, Cytomegalovirus
LLLQRGPQYSEHPTF

SEQ ID NO: 381 - protein, 15 aa, Cytomegalovirus
LLQRGPQYSEHPTFT

SEQ ID NO: 382 - protein, 15 aa, Cytomegalovirus
LQRGPQYSEHPTFTS

SEQ ID NO: 383 - protein, 15 aa, Cytomegalovirus
QRGPQYSEHPTFTSQ

SEQ ID NO: 384 - protein, 15 aa, Cytomegalovirus
RGPQYSEHPTFTSQY

SEQ ID NO: 385 - protein, 15 aa, Cytomegalovirus
GPQYSEHPTFTSQYR

SEQ ID NO: 386 - protein, 15 aa, Cytomegalovirus
PQYSEHPTFTSQYRI

SEQ ID NO: 387 - protein, 15 aa, Cytomegalovirus
QYSEHPTFTSQYRIQ

SEQ ID NO: 388 - protein, 15 aa, Cytomegalovirus
YSEHPTFTSQYRIQG

SEQ ID NO: 389 - protein, 15 aa, Cytomegalovirus
SEHPTFTSQYRIQGK

SEQ ID NO: 390 - protein, 15 aa, Cytomegalovirus
EHPTFTSQYRIQGKL

SEQ ID NO: 391 - protein, 15 aa, Cytomegalovirus
HPTFTSQYRIQGKLE

SEQ ID NO: 392 - protein, 15 aa, Cytomegalovirus
PTFTSQYRIQGKLEY

SEQ ID NO: 393 - protein, 15 aa, Cytomegalovirus
TFTSQYRIQGKLEYR

SEQ ID NO: 394 - protein, 15 aa, Cytomegalovirus
FTSQYRIQGKLEYRH

SEQ ID NO: 395 - protein, 15 aa, Cytomegalovirus
TSQYRIQGKLEYRHT

SEQ ID NO: 396 - protein, 15 aa, Cytomegalovirus
SQYRIQGKLEYRHTW

SEQ ID NO: 397 - protein, 15 aa, Cytomegalovirus
QYRIQGKLEYRHTWD

SEQ ID NO: 398 - protein, 15 aa, Cytomegalovirus
YRIQGKLEYRHTWDR

SEQ ID NO: 399 - protein, 15 aa, Cytomegalovirus
RIQGKLEYRHTWDRH

SEQ ID NO: 400 - protein, 15 aa, Cytomegalovirus
IQGKLEYRHTWDRHD

SEQ ID NO: 401 - protein, 15 aa, Cytomegalovirus
QGKLEYRHTWDRHDE

SEQ ID NO: 402 - protein, 15 aa, Cytomegalovirus
GKLEYRHTWDRHDEG

SEQ ID NO: 403 - protein, 15 aa, Cytomegalovirus
KLEYRHTWDRHDEGA

SEQ ID NO: 404 - protein, 15 aa, Cytomegalovirus
LEYRHTWDRHDEGAA

SEQ ID NO: 405 - protein, 15 aa, Cytomegalovirus
EYRHTWDRHDEGAAQ

SEQ ID NO: 406 - protein, 15 aa, Cytomegalovirus
YRHTWDRHDEGAAQG

SEQ ID NO: 407 - protein, 15 aa, Cytomegalovirus
RHTWDRHDEGAAQGD

SEQ ID NO: 408 - protein, 15 aa, Cytomegalovirus
HTWDRHDEGAAQGDD

SEQ ID NO: 409 - protein, 15 aa, Cytomegalovirus
TWDRHDEGAAQGDDD

SEQ ID NO: 410 - protein, 15 aa, Cytomegalovirus
WDRHDEGAAQGDDDV

SEQ ID NO: 411 - protein, 15 aa, Cytomegalovirus
DRHDEGAAQGDDDVW

SEQ ID NO: 412 - protein, 15 aa, Cytomegalovirus
RHDEGAAQGDDDVWT

SEQ ID NO: 413 - protein, 15 aa, Cytomegalovirus
HDEGAAQGDDDVWTS

SEQ ID NO: 414 - protein, 15 aa, Cytomegalovirus
DEGAAQGDDDVWTSG

SEQ ID NO: 415 - protein, 15 aa, Cytomegalovirus
EGAAQGDDDVWTSGS

SEQ ID NO: 416 - protein, 15 aa, Cytomegalovirus
GAAQGDDDVWTSGSD

SEQ ID NO: 417 - protein, 15 aa, Cytomegalovirus
AAQGDDDVWTSGSDS

SEQ ID NO: 418 - protein, 15 aa, Cytomegalovirus
AQGDDDVWTSGSDSD

SEQ ID NO: 419 - protein, 15 aa, Cytomegalovirus
QGDDDVWTSGSDSDE

SEQ ID NO: 420 - protein, 15 aa, Cytomegalovirus
GDDDVWTSGSDSDEE

SEQ ID NO: 421 - protein, 15 aa, Cytomegalovirus
DDDVWTSGSDSDEEL

SEQ ID NO: 422 - protein, 15 aa, Cytomegalovirus
DDVWTSGSDSDEELV

SEQ ID NO: 423 - protein, 15 aa, Cytomegalovirus
DVWTSGSDSDEELVT

SEQ ID NO: 424 - protein, 15 aa, Cytomegalovirus
VWTSGSDSDEELVTT

SEQ ID NO: 425 - protein, 15 aa, Cytomegalovirus
WTSGSDSDEELVTTE

SEQ ID NO: 426 - protein, 15 aa, Cytomegalovirus
TSGSDSDEELVTTER

SEQ ID NO: 427 - protein, 15 aa, Cytomegalovirus
SGSDSDEELVTTERK

SEQ ID NO: 428 - protein, 15 aa, Cytomegalovirus
GSDSDEELVTTERKT

SEQ ID NO: 429 - protein, 15 aa, Cytomegalovirus
SDSDEELVTTERKTP

SEQ ID NO: 430 - protein, 15 aa, Cytomegalovirus
DSDEELVTTERKTPR

SEQ ID NO: 431 - protein, 15 aa, Cytomegalovirus
SDEELVTTERKTPRV

SEQ ID NO: 432 - protein, 15 aa, Cytomegalovirus
DEELVTTERKTPRVT

SEQ ID NO: 433 - protein, 15 aa, Cytomegalovirus
EELVTTERKTPRVTG

SEQ ID NO: 434 - protein, 15 aa, Cytomegalovirus
ELVTTERKTPRVTGG

SEQ ID NO: 435 - protein, 15 aa, Cytomegalovirus
LVTTERKTPRVTGGG

SEQ ID NO: 436 - protein, 15 aa, Cytomegalovirus
VTTERKTPRVTGGGA

SEQ ID NO: 437 - protein, 15 aa, Cytomegalovirus
TTERKTPRVTGGGAM

SEQ ID NO: 438 - protein, 15 aa, Cytomegalovirus
TERKTPRVTGGGAMA

SEQ ID NO: 439 - protein, 15 aa, Cytomegalovirus
ERKTPRVTGGGAMAG

SEQ ID NO: 440 - protein, 15 aa, Cytomegalovirus
RKTPRVTGGGAMAGA

SEQ ID NO: 441 - protein, 15 aa, Cytomegalovirus
KTPRVTGGGAMAGAS

SEQ ID NO: 442 - protein, 15 aa, Cytomegalovirus
TPRVTGGGAMAGAST

SEQ ID NO: 443 - protein, 15 aa, Cytomegalovirus
PRVTGGGAMAGASTS

SEQ ID NO: 444 - protein, 15 aa, Cytomegalovirus
RVTGGGAMAGASTSA

SEQ ID NO: 445 - protein, 15 aa, Cytomegalovirus
VTGGGAMAGASTSAG

SEQ ID NO: 446 - protein, 15 aa, Cytomegalovirus
TGGGAMAGASTSAGR

SEQ ID NO: 447 - protein, 15 aa, Cytomegalovirus
GGGAMAGASTSAGRK

SEQ ID NO: 448 - protein, 15 aa, Cytomegalovirus
GGAMAGASTSAGRKR

SEQ ID NO: 449 - protein, 15 aa, Cytomegalovirus
GAMAGASTSAGRKRK

SEQ ID NO: 450 - protein, 15 aa, Cytomegalovirus
AMAGASTSAGRKRKS

SEQ ID NO: 451 - protein, 15 aa, Cytomegalovirus
MAGASTSAGRKRKSA

SEQ ID NO: 452 - protein, 15 aa, Cytomegalovirus
AGASTSAGRKRKSAS

SEQ ID NO: 453 - protein, 15 aa, Cytomegalovirus
GASTSAGRKRKSASS

SEQ ID NO: 454 - protein, 15 aa, Cytomegalovirus
ASTSAGRKRKSASSA

SEQ ID NO: 455 - protein, 15 aa, Cytomegalovirus
STSAGRKRKSASSAT

SEQ ID NO: 456 - protein, 15 aa, Cytomegalovirus
TSAGRKRKSASSATA

SEQ ID NO: 457 - protein, 15 aa, Cytomegalovirus
SAGRKRKSASSATAC

SEQ ID NO: 458 - protein, 15 aa, Cytomegalovirus
AGRKRKSASSATACT

SEQ ID NO: 459 - protein, 15 aa, Cytomegalovirus
GRKRKSASSATACTS

SEQ ID NO: 460 - protein, 15 aa, Cytomegalovirus
RKRKSASSATACTSG

SEQ ID NO: 461 - protein, 15 aa, Cytomegalovirus
KRKSASSATACTSGV

SEQ ID NO: 462 - protein, 15 aa, Cytomegalovirus
RKSASSATACTSGVM

SEQ ID NO: 463 - protein, 15 aa, Cytomegalovirus
KSASSATACTSGVMT

SEQ ID NO: 464 - protein, 15 aa, Cytomegalovirus
SASSATACTSGVMTR

SEQ ID NO: 465 - protein, 15 aa, Cytomegalovirus
ASSATACTSGVMTRG

SEQ ID NO: 466 - protein, 15 aa, Cytomegalovirus
SSATACTSGVMTRGR

SEQ ID NO: 467 - protein, 15 aa, Cytomegalovirus
SATACTSGVMTRGRL

SEQ ID NO: 468 - protein, 15 aa, Cytomegalovirus
ATACTSGVMTRGRLK

SEQ ID NO: 469 - protein, 15 aa, Cytomegalovirus
TACTSGVMTRGRLKA

SEQ ID NO: 470 - protein, 15 aa, Cytomegalovirus
ACTSGVMTRGRLKAE

SEQ ID NO: 471 - protein, 15 aa, Cytomegalovirus
CTSGVMTRGRLKAES

SEQ ID NO: 472 - protein, 15 aa, Cytomegalovirus
TSGVMTRGRLKAEST

SEQ ID NO: 473 - protein, 15 aa, Cytomegalovirus
SGVMTRGRLKAESTV

SEQ ID NO: 474 - protein, 15 aa, Cytomegalovirus
GVMTRGRLKAESTVA

SEQ ID NO: 475 - protein, 15 aa, Cytomegalovirus
VMTRGRLKAESTVAP

SEQ ID NO: 476 - protein, 15 aa, Cytomegalovirus
MTRGRLKAESTVAPE

SEQ ID NO: 477 - protein, 15 aa, Cytomegalovirus
TRGRLKAESTVAPEE

SEQ ID NO: 478 - protein, 15 aa, Cytomegalovirus
RGRLKAESTVAPEED

SEQ ID NO: 479 - protein, 15 aa, Cytomegalovirus
GRLKAESTVAPEEDT

SEQ ID NO: 480 - protein, 15 aa, Cytomegalovirus
RLKAESTVAPEEDTD

SEQ ID NO: 481 - protein, 15 aa, Cytomegalovirus
LKAESTVAPEEDTDE

SEQ ID NO: 482 - protein, 15 aa, Cytomegalovirus
KAESTVAPEEDTDED

SEQ ID NO: 483 - protein, 15 aa, Cytomegalovirus
AESTVAPEEDTDEDS

SEQ ID NO: 484 - protein, 15 aa, Cytomegalovirus
ESTVAPEEDTDEDSD

SEQ ID NO: 485 - protein, 15 aa, Cytomegalovirus
STVAPEEDTDEDSDN

SEQ ID NO: 486 - protein, 15 aa, Cytomegalovirus
TVAPEEDTDEDSDNE

SEQ ID NO: 487 - protein, 15 aa, Cytomegalovirus
VAPEEDTDEDSDNEI

SEQ ID NO: 488 - protein, 15 aa, Cytomegalovirus
APEEDTDEDSDNEIH

SEQ ID NO: 489 - protein, 15 aa, Cytomegalovirus
PEEDTDEDSDNEIHN

SEQ ID NO: 490 - protein, 15 aa, Cytomegalovirus
EEDTDEDSDNEIHNP

SEQ ID NO: 491 - protein, 15 aa, Cytomegalovirus
EDTDEDSDNEIHNPA

SEQ ID NO: 492 - protein, 15 aa, Cytomegalovirus
DTDEDSDNEIHNPAV

SEQ ID NO: 493 - protein, 15 aa, Cytomegalovirus
TDEDSDNEIHNPAVF

SEQ ID NO: 494 - protein, 15 aa, Cytomegalovirus
DEDSDNEIHNPAVFT

SEQ ID NO: 495 - protein, 15 aa, Cytomegalovirus
EDSDNEIHNPAVFTW

SEQ ID NO: 496 - protein, 15 aa, Cytomegalovirus
DSDNEIHNPAVFTWP

SEQ ID NO: 497 - protein, 15 aa, Cytomegalovirus
SDNEIHNPAVFTWPP

SEQ ID NO: 498 - protein, 15 aa, Cytomegalovirus
DNEIHNPAVFTWPPW

SEQ ID NO: 499 - protein, 15 aa, Cytomegalovirus
NEIHNPAVFTWPPWQ

SEQ ID NO: 500 - protein, 15 aa, Cytomegalovirus
EIHNPAVFTWPPWQA

SEQ ID NO: 501 - protein, 15 aa, Cytomegalovirus
IHNPAVFTWPPWQAG

SEQ ID NO: 502 - protein, 15 aa, Cytomegalovirus
HNPAVFTWPPWQAGI

SEQ ID NO: 503 - protein, 15 aa, Cytomegalovirus
NPAVFTWPPWQAGIL

SEQ ID NO: 504 - protein, 15 aa, Cytomegalovirus
PAVFTWPPWQAGILA

SEQ ID NO: 505 - protein, 15 aa, Cytomegalovirus
AVFTWPPWQAGILAR

SEQ ID NO: 506 - protein, 15 aa, Cytomegalovirus
VFTWPPWQAGILARN

SEQ ID NO: 507 - protein, 15 aa, Cytomegalovirus
FTWPPWQAGILARNL

SEQ ID NO: 508 - protein, 15 aa, Cytomegalovirus
TWPPWQAGILARNLV

SEQ ID NO: 509 - protein, 15 aa, Cytomegalovirus
WPPWQAGILARNLVP

SEQ ID NO: 510 - protein, 15 aa, Cytomegalovirus
PPWQAGILARNLVPM

SEQ ID NO: 511 - protein, 15 aa, Cytomegalovirus
PWQAGILARNLVPMV

SEQ ID NO: 512 - protein, 15 aa, Cytomegalovirus
WQAGILARNLVPMVA

SEQ ID NO: 513 - protein, 15 aa, Cytomegalovirus
QAGILARNLVPMVAT

SEQ ID NO: 514 - protein, 15 aa, Cytomegalovirus
AGILARNLVPMVATV

SEQ ID NO: 515 - protein, 15 aa, Cytomegalovirus
GILARNLVPMVATVQ

SEQ ID NO: 516 - protein, 15 aa, Cytomegalovirus
ILARNLVPMVATVQG

SEQ ID NO: 517 - protein, 15 aa, Cytomegalovirus
LARNLVPMVATVQGQ

SEQ ID NO: 518 - protein, 15 aa, Cytomegalovirus
ARNLVPMVATVQGQN

SEQ ID NO: 519 - protein, 15 aa, Cytomegalovirus
RNLVPMVATVQGQNL

SEQ ID NO: 520 - protein, 15 aa, Cytomegalovirus
NLVPMVATVQGQNLK

SEQ ID NO: 521 - protein, 15 aa, Cytomegalovirus
LVPMVATVQGQNLKY

SEQ ID NO: 522 - protein, 15 aa, Cytomegalovirus
VPMVATVQGQNLKYQ

SEQ ID NO: 523 - protein, 15 aa, Cytomegalovirus
PMVATVQGQNLKYQE

SEQ ID NO: 524 - protein, 15 aa, Cytomegalovirus
MVATVQGQNLKYQEF

SEQ ID NO: 525 - protein, 15 aa, Cytomegalovirus
VATVQGQNLKYQEFF

SEQ ID NO: 526 - protein, 15 aa, Cytomegalovirus
ATVQGQNLKYQEFFW

SEQ ID NO: 527 - protein, 15 aa, Cytomegalovirus
TVQGQNLKYQEFFWD

SEQ ID NO: 528 - protein, 15 aa, Cytomegalovirus
VQGQNLKYQEFFWDA

SEQ ID NO: 529 - protein, 15 aa, Cytomegalovirus
QGQNLKYQEFFWDAN

SEQ ID NO: 530 - protein, 15 aa, Cytomegalovirus
GQNLKYQEFFWDAND

SEQ ID NO: 531 - protein, 15 aa, Cytomegalovirus
QNLKYQEFFWDANDI

SEQ ID NO: 532 - protein, 15 aa, Cytomegalovirus
NLKYQEFFWDANDIY

SEQ ID NO: 533 - protein, 15 aa, Cytomegalovirus
LKYQEFFWDANDIYR

SEQ ID NO: 534 - protein, 15 aa, Cytomegalovirus
KYQEFFWDANDIYRI

SEQ ID NO: 535 - protein, 15 aa, Cytomegalovirus
YQEFFWDANDIYRIF

SEQ ID NO: 536 - protein, 15 aa, Cytomegalovirus
QEFFWDANDIYRIFA

SEQ ID NO: 537 - protein, 15 aa, Cytomegalovirus
EFFWDANDIYRIFAE

SEQ ID NO: 538 - protein, 15 aa, Cytomegalovirus
FFWDANDIYRIFAEL

SEQ ID NO: 539 - protein, 15 aa, Cytomegalovirus
FWDANDIYRIFAELE

SEQ ID NO: 540 - protein, 15 aa, Cytomegalovirus
WDANDIYRIFAELEG

SEQ ID NO: 541 - protein, 15 aa, Cytomegalovirus
DANDIYRIFAELEGV

SEQ ID NO: 542 - protein, 15 aa, Cytomegalovirus
ANDIYRIFAELEGVW

SEQ ID NO: 543 - protein, 15 aa, Cytomegalovirus
NDIYRIFAELEGVWQ

SEQ ID NO: 544 - protein, 15 aa, Cytomegalovirus
DIYRIFAELEGVWQP

SEQ ID NO: 545 - protein, 15 aa, Cytomegalovirus
IYRIFAELEGVWQPA

SEQ ID NO: 546 - protein, 15 aa, Cytomegalovirus
YRIFAELEGVWQPAA

SEQ ID NO: 547 - protein, 15 aa, Cytomegalovirus
RIFAELEGVWQPAAQ

SEQ ID NO: 548 - protein, 15 aa, Cytomegalovirus
IFAELEGVWQPAAQP

SEQ ID NO: 549 - protein, 15 aa, Cytomegalovirus
FAELEGVWQPAAQPK

SEQ ID NO: 550 - protein, 15 aa, Cytomegalovirus
AELEGVWQPAAQPKR

SEQ ID NO: 551 - protein, 15 aa, Cytomegalovirus
ELEGVWQPAAQPKRR

SEQ ID NO: 552 - protein, 15 aa, Cytomegalovirus
LEGVWQPAAQPKRRR

SEQ ID NO: 553 - protein, 15 aa, Cytomegalovirus
EGVWQPAAQPKRRRH

SEQ ID NO: 554 - protein, 15 aa, Cytomegalovirus
GVWQPAAQPKRRRHR

SEQ ID NO: 555 - protein, 15 aa, Cytomegalovirus
VWQPAAQPKRRRHRQ

SEQ ID NO: 556 - protein, 15 aa, Cytomegalovirus
WQPAAQPKRRRHRQD

SEQ ID NO: 557 - protein, 15 aa, Cytomegalovirus
QPAAQPKRRRHRQDA

SEQ ID NO: 558 - protein, 15 aa, Cytomegalovirus
PAAQPKRRRHRQDAL

SEQ ID NO: 559 - protein, 15 aa, Cytomegalovirus
AAQPKRRRHRQDALP

SEQ ID NO: 560 - protein, 15 aa, Cytomegalovirus
AQPKRRRHRQDALPG

SEQ ID NO: 561 - protein, 15 aa, Cytomegalovirus
QPKRRRHRQDALPGP

SEQ ID NO: 562 - protein, 15 aa, Cytomegalovirus
PKRRRHRQDALPGPC

SEQ ID NO: 563 - protein, 15 aa, Cytomegalovirus
KRRRHRQDALPGPCI

SEQ ID NO: 564 - protein, 15 aa, Cytomegalovirus
RRRHRQDALPGPCIA

SEQ ID NO: 565 - protein, 15 aa, Cytomegalovirus
RRHRQDALPGPCIAS

SEQ ID NO: 566 - protein, 15 aa, Cytomegalovirus
RHRQDALPGPCIAST

SEQ ID NO: 567 - protein, 15 aa, Cytomegalovirus
HRQDALPGPCIASTP

SEQ ID NO: 568 - protein, 15 aa, Cytomegalovirus
RQDALPGPCIASTPK

SEQ ID NO: 569 - protein, 15 aa, Cytomegalovirus
QDALPGPCIASTPKK

SEQ ID NO: 570 - protein, 15 aa, Cytomegalovirus
DALPGPCIASTPKKH

SEQ ID NO: 571 - protein, 15 aa, Cytomegalovirus
ALPGPCIASTPKKHR

SEQ ID NO: 572 - protein, 15 aa, Cytomegalovirus
LPGPCIASTPKKHRG

SEQ ID NO: 573 - protein, 9 aa, Cytomegalovirus
NLVPMVATV

SEQ ID NO: 574 - protein, 9 aa, Cytomegalovirus
ILKEPVHGV

SEQ ID NO: 575 - protein, 9 aa, Cytomegalovirus
RIFAELEGV

SEQ ID NO: 576 - protein, 9 aa, Cytomegalovirus
YTPDSTPCH

SEQ ID NO: 577 - protein, 21 aa, Cytomegalovirus
LILVSQYTPDSTPCHRGDNQL

SEQ ID NO: 578 - protein, 16 aa, Cytomegalovirus
DEDSDNEIHNPAVFTW

SEQ ID NO: 579 - protein, 29 aa, Cytomegalovirus
PSLILVSQYTPDSTPCHRGDNQLQVQHTR

SEQ ID NO: 580 - protein, 23 aa, Cytomegalovirus
QWKEPDVYYTSAFVFPTKDVALR

SEQ ID NO: 581 - protein, 22 aa, Cytomegalovirus
LLQRGPQYSEHPTFTSQYRIQG

SEQ ID NO: 582 - protein, 20 aa, Cytomegalovirus
YRHTWDRHDEGAAQGDDDVW

SEQ ID NO: 583 - protein, 24 aa, Cytomegalovirus
TSGVMTRGRLKAESTVAPEEDTDE

SEQ ID NO: 584 - protein, 21 aa, Cytomegalovirus
QGQNLKYQEFFWDANDIYRIF

(19) United States
(12) Patent Application Publication
(10) Pub. No.: US 2005/0221381 A1
(43) Pub. Date: Oct. 6, 2005

(54) Method for isolating ligands

(76) Inventors: Christof Klade, Wiener Neustadt (AT); Juliane Schalich, The Hague (NL); Oresta Vytvytska, Vienna (AT); Wolfgang Zauner, Vienna (AT); Max Birnstiel, Lugaggia (CH); Gerald Aichinger, Vienna (AT); Alexander Otava, Vienna (AT); Frank Mattner, Vienna (AT)

Correspondence Address: FULBRIGHT & JAWORSKI L.L.P., 600 CONGRESS AVE., SUITE 2400, AUSTIN, TX, 78701, US

(21) Appl. No.: 10/505,929

(22) PCT Filed: Feb. 27, 2003
(86) PCT No.: PCT/EP03/02005

§ 371 Date: Mar. 10, 2005

(30) Foreign Application Priority Data
Feb. 28, 2002 (AT) 316/2002
Sep. 11, 2002 (AT) 1376/2002

(51) Int. Cl. C07K 16/28; C07K 14/74; G01N 33/53

(52) U.S. Cl. 435/7.1; 530/350; 530/388.22

(57) ABSTRACT

Described is a method for isolating ligands which have a binding capacity to a MHC/HLA molecule or a complex comprising said ligand and said MHC/HLA molecule which method comprises the following steps:—providing a pool of ligands, said pool containing ligands which bind to said MHC/HLA molecule and ligands which do not bind to said MHC/HLA molecule,—contacting said MHC/HLA molecule with said pool of ligands whereby a ligand which has a binding capacity to said MHC/HLA molecule binds to said MHC/HLA molecule and a complex comprising said ligand and said MHC/HLA molecule is formed,—detecting and optionally separating said complex from the ligands which do not bind to said MHC/HLA molecule and—optionally isolating and characterising the ligand from said complex as well as a method for isolating T cell epitopes which have a binding capacity to a MHC/HLA molecule.

### CROSS-REFERENCE TO RELATED APPLICATIONS

This application is a U.S. national phase application under 35 U.S.C. § 371 of International Application No. PCT/EP03/02005 filed 27 Feb. 2003, which claims priority to Austrian Applications No. A 316/2002 filed 28 Feb. 2002 and A 1376/2002 filed 13 Sep. 2002. The entire contents of each of the above-referenced disclosures is incorporated herein by reference.

### BACKGROUND OF THE INVENTION

The present invention relates to a method for isolating ligands, especially for isolating T cell epitopes which have a binding capacity to a MHC/HLA molecule.

The immune system is a complex network of inter-related cell types and molecules, which has evolved in order to protect multicellular organisms from infectious microorganisms. It can be divided into the evolutionary older innate (or natural) immunity and adaptive (or acquired) immunity. The innate immune system recognizes patterns, which are usually common and essential for pathogens. For this limited number of molecular structures germ-line encoded receptors have evolved. By contrast, cells of the adaptive immune system—B and T lymphocytes—can recognize a huge variety of antigenic structures. The receptors, termed according to the cell types expressing them, B cell receptor (BCR, its soluble versions are called antibodies) and T cell receptor (TCR, only cell-surface associated forms) are generated by somatic recombination and show a clonal distribution. Thus, initially there is only small number of cells with a certain specificity. Upon antigen encounter these cells start to divide (clonal expansion) to generate an effector population able to cope with the antigen. After elimination of antigen a specialized sub-population of cells specifically recognizing this antigen remains as immunological memory. Taken together the adaptive immune system is slow (compared to innate immunity), however specific and it improves upon repeated exposure to a given pathogen/antigen.

T cells have a central role in adaptive immunity. Their receptors (TCRs) recognize “major histocompatibility complex” (MHC or HLA):peptide complexes on the surface of cells. These peptides are called T cell epitopes and represent degradation products of antigens. There are two major classes of T cells: CD8-positive cytotoxic T cells (CTL) are restricted to MHC class I. CD4-positive helper T cells (HTL) are restricted to MHC class II. HTL are essential for many features of adaptive immunity: activation of so called “professional antigen-presenting cells” (APCs), immunoglobulin (Ig) class switch, the germinal center reaction and Ig affinity maturation, activation of CTL, immunological memory, regulation of the immune response and others.

MHC molecules collect peptides inside the cell and present them on the cell surface to TCRs of T cells. There are two major classes of MHC, class I recognized by CD8-positive CTL and class II recognized by CD4-positive HTL.

MHC class I molecules consist of a membrane-anchored alpha-chain of 45 kDa and the non-covalently attached b2-microglobulin (b2m) of 12 kDA. Resolution of the 3-dimensional structure by X-ray crystallography (Stern and Wiley 1994) revealed that the alpha-chain possesses a cleft, which is closed at both ends and accommodates peptides from 8 to 11 amino acids length. Class I molecules are ubiquitously expressed, and the peptides they present originate from cytoplasmic proteins. These are degraded by the proteasome, and the resulting peptides are actively transported into the endoplasmatic reticulum (ER). There, with the help of several chaperones, MHC:peptide complexes are formed and transported to the cell surface (Heemels 1995). Thus, MHC class I mirrors the proteome of a cell on its surface and allows T cells to recognize intracellular pathogens or malignant cells.

MHC class II molecules consist of two membrane-anchored proteins (alpha- and beta-chain) of 35 kDa and 30 kDa, respectively. These together form a cleft, open at both ends, which can accommodate peptides of variable length, usually from 12 to 25 amino acids. Despite these differences, class I and II molecules share surprising structural similarity (Stern and Wiley 1994). Class II molecules are only expressed on professional APC including dendritic cells (DC), B-cells and macrophages/monocytes. These cells are specialized in taking up and processing antigens in the endosomal pathway. Immediately after their biosynthesis, class II molecules are complexed by the so-called invariant chain (Ii), which prevents binding of peptides in the ER. When vesicles containing class II:Ii complexes fuse with endosomes containing degradation products of exogenous antigen, Ii is degraded until the MHC binding cleft is only complexed by the so-called CLIP peptide. The latter is with the help of chaperones like HLA-DM exchanged by antigenic peptides (Villadangos 2000). Finally, MHC:peptide complexes are again presented on the surface of APCs, which interact in numerous ways with HTL.

Being both polygenic and extremely polymorphic, the MHC system is highly complex. For the class 1 alpha-chain in humans there are three gene loci termed HLA-A, -B and -C. Likewise, there are three class II alpha-chain loci (DRA, DQA, DPA); for class II beta-chain loci the situation is even more complex as there are four different DR beta-chains (DRB1,2,3,5) plus DQB and DPB. Except the monomorphic DR alpha-chain DRA, each gene locus is present in many different alleles (dozens to hundreds) in the population (Klein 1986). Different alleles have largely distinct binding specificities for peptides. Alleles are designated, for example, HLA-A*0201 or HLA-DRB1*0401 or HLA-DPA*0101/DPB*0401.

T cell epitopes have been identified by a variety of approaches (Van den Eynde 1997). T cell lines and clones have for instance been used to screen cDNA expression libraries for instance in the context of COS cells transfected with the appropriate HLA-molecule. Alternatively, biochemical approaches have been pursued. The latter involved elution of natural ligands from MHC molecules on the surface of target cells, the separation of these peptides by several chromatography steps, analysis of their reactivity with lymphocytes in epitope reconstitution assays and sequencing by mass spectrometry (Wölfel et al. 1994, Cox et al. 1994).

Recently the advent of highly sensitive cytokine detection assays like the IFN-γ ELIspot allowed using lymphocytes directly ex vivo for screening of overlapping synthetic peptides (Maecker 2001, Kern 2000, Tobery 2001). Primarily, Kern et al. (1999&2000) used arrays of pools of overlapping 9mer peptides to map CD8+ T cell epitopes in vitro. Later, Tobery et al., 2001 modified this approach and demonstrated that pools containing as many as 64 20mer peptides may be used to screen for both CD8+ and CD4+ T cell epitopes in mice. Both these methods were based on the monitoring of antigen-specific response by measuring INF-gamma production either by intracellular staining (Kern et al 2000) or in ELIspot assay (Tobery et al., 2001). By use of mixtures of 15-mers the CD4+ T cell responses are approximately equal to those detected when whole soluble protein was used as an antigen, while—not surprising—the CD8+ T cell responses are significantly higher than the often negligible responses detected with soluble protein stimulation. Furthermore, the CD8+ T cell responses to a mixture of 15 amino acid peptides are similar to those obtained with a mix of 8-12 amino acid peptides, selected to represent known MHC class I minimal epitopes. Most probably peptidases associated with the cell membrane are responsible for “clipping” peptides to optimal length under these circumstances (Maecker et al, 2001).

An interesting alternative is to screen synthetic combinatorial peptide libraries with specific lymphocytes. For instance, a decapeptide library consisting of 200 mixtures arranged in a positional scanning format, has been successfully used for identification of peptide ligands that stimulate clonotypic populations of T cells (Wilson, et al., J. Immunol., 1999, 163:6424-6434).

Many T cell epitopes have been identified by so called “Reverse immunological approaches” Rammensee 1999). In this case the protein giving rise to a potential T cell epitope is known, and its primary sequence is scanned for HLA binding motifs. Typically dozens to hundreds of candidate peptides or even a full set of overlapping peptides are synthesized and tested for binding to HLA molecules. Usually, the best binders are selected for further characterization with regard to their reactivity with T cells. This can for instance be done by priming T cells in vitro or in vivo with the help of HLA transgenic mice.

### SUMMARY OF THE INVENTION

It is an object of the present invention to provide a method for screening ligands for specific MHC molecules, preferably for delivering suitable and specific T cell epitopes selected from a variety of ligands having unknown specificity for a given MHC molecule.

Therefore the present invention provides a method for isolating ligands which have a binding capacity to a MHC/HLA molecule or a complex comprising said ligand and said MHC/HLA molecule which method comprises the following steps:
providing a pool of ligands, said pool containing ligands which bind to said MHC/HLA molecule and ligands which do not bind to said MHC/HLA molecule,
contacting said MHC/HLA molecule with said pool of ligands whereby a ligand which has a binding capacity to said MHC/HLA molecule binds to said MHC/HLA molecule and a complex comprising said ligand and said MHC/HLA molecule is formed,
detecting and optionally separating said complex from the ligands which do not bind to said MHC/HLA molecule and
optionally isolating and characterising the ligand from said complex.

The present invention also provides a method for isolating T cell epitopes which have a binding capacity to a MHC/HLA molecule or a complex comprising said epitope and said MHC/HLA molecule which method comprises the following steps:
providing a pool of ligands, said pool containing ligands which bind to a MHC/HLA molecule and ligands which do not bind to said MHC/HLA molecule,
contacting said MHC/HLA molecule with said pool of ligands whereby a ligand which has a binding capacity to said MHC/HLA molecule binds to said MHC/HLA molecule and a complex comprising said ligand and said MHC/HLA molecule is formed,
detecting and optionally separating said complex from the ligands which do not bind to said MHC/HLA molecule,
optionally isolating and characterising the ligand from said complex,
assaying said optionally isolated ligand or said complex in a T cell assay for T cell activation capacity and
providing the optionally isolated ligand with a T cell activation capacity as T cell epitope or as complex.

The method according to the present invention enables a screening system for screening binding capacity to specific MHC/HLA molecules. Identifying MHC binding molecules is an important tool for molecular characterisation of pathogens, tumors, etc. It is therefore possible with the present invention to screen a variety (a “pool”) of potential ligands at once for their functional affinity towards MHC molecules. Binding affinity towards MHC molecules is also a necessary prerequisite for ligands intended to be used as T cell epitopes, although not a sufficient one. Suitable T cell epitope candidates have also to be screened and assayed with respect to their T cell activation capacity. The combination of the screening method for binding according to the present invention with a suitable T cell assay therefore provides the method for isolating T cell epitopes according to the present invention wherein such T cell epitopes are identifyable out of a pool of potential ligands using an MHC binding assay.

In contrast to the prior art, where such assays have always been performed on ligands with known binding/MHC specificity, the methods according to the present invention provide such assays as a screening tool for pools with ligands of unknown specificity. In the prior art such assays have been typically performed on individual single ligands, to test their binding affinity to MHC/HLA molecules. In Kwok et al. (2001) pools of maximally up to 5 overlapping synthetic peptides were used to generate MHC class II tetramers; the latter were then used to stain PBMC for T cells specific for particular MHC class II:peptide complexes which were generated in the binding reaction with the pools of 5 peptides. However, an increase in the number of ligands per pool in such an approach was not regarded as being possible, both for sensitivity and specificity reasons (Novak et al. 2001). A problem with regard to specificity would be the generation of MHC tetramers with more then one binder per tetramer, if more than one binder would be present in the pool. This would preclude staining of T cells, which is used for identification of epitopes in the approach described in the prior art. In strong contrast to that the approach according to the present invention allows the identification of more than on binder out of highly complex mixtures containing more than one binder.

The nature of the pool to be screened with the present invention is not critical: the pools may contain any naturally or not naturally occurring substance which a) binds specifically to MHC/HLA molecules and/or b) may be specifically recognized by T cells. The binding properties of the set of ligands of the pool with respect to MHC molecules is not known; therefore, usually binders and at least a non-binder for a given MHC molecule are contained in the pool. The pool therefore comprises at least ten different ligands. Practically, pools are used according to the present invention containing significantly more different ligand species, e.g. 20 or more, 100 or more, 1.000 or more or 10.000 or more. It is also possible to screen larger libraries (with e.g. more then 10⁶, more than 10⁸ or even more than 10¹⁰ different ligand species). This, however, is mainly dependent on the availability of such ligand libraries.

Evidently, MHC:peptide complexes are not typical receptor-ligand systems and have hitherto not been regarded as being the basis of a proper screening tool. In vivo, usually only MHC:peptide complexes but not empty MHC molecules exist. MHC:peptide complexes are not generated by a simple binding of a peptide to an empty MHC molecule, but through the highly complex—and still not fully understood—process of so called “antigen processing and presentation”. This is a highly organized intracellular process involving multiple enzymes (cytosolic and lysosomal proteases, transporters, chaperones, peptide-exchange factors, etc.). In fact it is well known that MHC molecules without ligand are unstable and undergo rapid degradation.

Therefore, a main feature of the present invention is the development of production, purification and reaction conditions providing recombinant “empty” MHC-molecules and enabling their use to “capture” ligands” from pools. There are no examples in the prior art demonstrating a similar possibility. The above cited Novak et al. reference discloses a production (insect cells) and purification strategy to obtain recombinant MHC molecules, which are subsequently incubated with a few peptides and tetramerized. The MHC tetramers are used to stain cells from individuals who are likely to have T-cells against the antigen represented by the peptides, thus providing the necessary proof, that the ligand represents also a true T-cell epitope. However, the mentioned prior art explicitly states that only up to 5 peptides could be used successfully. This is in strong contrast to the present approach, which may be successfully applied for 10, 21 and even several hundreds or thousands of peptides per pool.

Preferred pools of ligands to be used in the method according to the present invention are selected from the group consisting of a pool of peptides, especially overlapping peptides, a pool of protein fragments, a pool of glycolipids, a pool of glycosphingolipids, a pool of lipopeptides, a pool of lipids, a pool of glycans, a pool of modified peptides, a pool obtained from antigen-presenting cells, preferably in the form of total lysates or fractions thereof, especially fractions eluted from the surface or the MHC/HLA molecules of these cells, a pool comprised of fragments of cells, especially pathogen cells, tumor cells or tissues, a pool comprised of peptide libraries, pools of (poly)-peptides generated from recombinant DNA libraries, especially derived from pathogens or tumor cells, a pool of proteins and/or protein fragments from a specific pathogen or mixtures thereof.

The ligands of the pools may be derived from natural sources (in native and/or derivatised form) but also be produced synthetically (e.g. by chemical sysntesis or by recombinant technology). If (poly)peptide ligands are provided in the pools, those peptides are preferably generated by peptide synthesizers or by recombinant technology. According to a preferred embodiment, a pool of (poly)peptides may be generated from recombinant DNA libraries, e.g. derived from pathogens or tumor cells, by in vitro translation (e.g. by ribosome display) or by expression through heterologous hosts like E. coli or others.

Ligands are therefore preferably peptides being fragments of antigens which could serve as T cell epitopes. Such peptides should preferably be longer than 6, especially longer than 8 amino acids and have preferred maximum lengths of 40, 30, 20, 15 or even 11 or 12 amino acids.

Preferred pathogens wherefrom such peptides can be taken are selected from human immune deficiency virus (HIV), hepatitis A and B viruses, hepatitis C virus (HCV), Rous sarcoma virus (RSV), Epstein Barr virus (EBV), Influenza virus, Rotavirus, Staphylococcus aureus, Chlamydia pneumoniae, Chlamydia trachomatis, Mycobacterium tuberculosis, Streptococcus pneumoniae, Bacillus antracis, Vibrio cholerae, Plasmodium sp. (Pl. falciparum, Pl. vivax, etc.), Aspergillus sp. or Candida albicans. Antigens may also be molecules expressed by cancer cells (tumor antigens). In the same way also tumor antigens (cancer vaccines) or autoimmune antigens may be used for providing suitable (peptide) ligands for the present invention.

The nature of the specific MHC molecules (of course also MHC-like molecules are encompassed by this term) to be selected for the present methods is again not critical. Therefore, these molecules may be selected in principle from any species, especially primates like humans (HLA, see below), chimpanzees, other mammals, e.g. maquaques, rabbits, cats, dogs or rodents like mice, rats, guinea pigs and others, agriculturally important animals like cattle, horses, sheep and fish, although human (or “humanized”) molecules are of course preferred for providing vaccines for humans. For providing vaccines for specific animals, especially agriculturally important animals, like cattle, horses, sheep and fish, the use of MHC molecules being specific for these animals is preferred.

Preferred HLA molecules therefore comprise Class I molecules derived from the HLA-A, -B or -C loci, especially A1, A2, A3, A24, A11, A23, A29, A30, A68; B7, B8, B15, B16, B27, B35, B40, B44, B46, B51, B52, B53; Cw3, Cw4, Cw6, Cw7; Class II molecules derived from the HLA-DP, -DQ or -DR loci, especially DR1, DR2, DR3, DR4, DR7, DR8, DR9, DR11, DR12, DR13, DR51, DR52, DR53; DP2, DP3, DP4; DQ1, DQ3, DQ5, DQ6; and non-classical MHC/HLA and MHC/HLA-like molecules, which can specifically bind ligands, especially HLA-E, HLA-G, MICA, MICB, Qa1, Qa2, T10, T18, T22, M3 and members of the CD1 family.

According to a preferred embodiment, the methods according to the present invention is characterised in that said MHC/HLA molecules are selected from HLA class I molecules, HLA class II molecules, non classical MHC/HLA and MHC/HLA-like molecules or mixtures thereof, or mixtures thereof.

Preferably, the optional characterising step of the ligands of the complex is performed by using a method selected from the group consisting of mass spectroscopy, polypeptide sequencing, binding assays, especially SDS-stability assays, identification of ligands by determination of their retention factors by chromatography, especially HPLC, or other spectroscopic techniques, especially violet (UV), infra-red (IR), nuclear magnetic resonance (NMR), circular dichroism (CD) or electron spin resonance (ESR), or combinations thereof.

According to a preferred embodiment the method of the present invention is characterised in that it is combined with a cytokine secretion assay, preferably with an Elispot assay, an intracellular cytokine staining, FACS or an ELISA (enzyme-linked immunoassays) (see e.g. Current Protocols in Immunology).

Preferred T cell assays comprise the mixing and incubation of said complex with isolated T cells and subsequent measuring cytokine secretion or proliferation of said isolated T cells and/or the measuring up-regulation of activation markers, especially CD69, CD38, or down-regulation of surface markers, especially CD3, CD8 or TCR and/or the measuring up-/down-regulation of mRNAs involved in T cell activation, especially by real-time RT-PCR (see e.g. Current Protocols in Immunology, Current Protocols in Molecular Biology). The T cell activation capacity tests according to the present invention (herein referred to as “T cell assay”) may preferably also be realised in transgenic mice, especially with suitably designed human MHC/HLA set up (e.g. having one or more human MHC/HLA molecules integrated in their genome).

Further preferred T cell assays are selected from T cell assays measuring phosphorylation/de-phosphorylation of components downstream of the T cell receptor, especially p56 lck, ITAMS of the TCR and the zeta chain, ZAP70, LAT, SLP-76, fyn, and lyn, T cell assays measuring intracellular Ca++ concentration or activation of Ca++-dependent proteins, T cell assays measuring formation of immunological synapses, T cell assays measuring release of effector molecules, especially perforin, granzymes or granulolysin or combinations of such T cell assays (see e.g. Current Protocols in Immunology, Current Protocols in Cell Biology).

WO 00/31542 presents methods for identifying antigens exclusively from tumor cells. The antigenic peptides are extracted of from the MHC-peptide complexes located on the surface of hapten-modified malignant cells. Then haptenized peptides can be separated by hapten-specific affinity chromatography and sequenced. The embodiment of this invention is that peptides originally isolated from MHC molecules located on the surface of tumor cells have the property of stimulating T cells. Stimulation refers to T cell proliferation in response to the addition of cell extract, as well as production of cytokines such as INF-gamma, TNF, IL-2 and others. Based on the sequence of isolated peptide it is possible to identify the source of antigen.

Although the net result of this approach is also a T-cell epitope, this process substantially differs from the present invention: in the prior art natural MHC:peptide complexes are isolated from cellular systems, in the present invention purified, recombinant MHC molecules are used to isolate ligands from any source, cellular or synthetic, natural or artifical; in the prior art haptenization of the epitope is required for subsequent isolation by hapten-affinity chromatography, whereas the present invention is independent of such a step.

Tana et al. (1998) describe the approach for screening of antigenic peptides recognized by T cells from synthetic combinatorial peptide library designed on the base of the known binding motifs for set type of MHC molecules. Thus, the number of peptides to be screened is essentially reduced (˜10³) comparing with a “comprehensive” library consisting of 20⁹ all possible peptides. The peptides are combined in mixtures of nine peptides containing different amino acids in two fixed positions. Then the mixtures have been examined for eliciting T cell proliferative response. Thus, this approach is restricted for detection of amino acid residues appropriate for binding to the set MHC molecules and recognition by TCR receptors.

In Bitmansour et al. (2001) the matrix approach previously described by Kern et al., (1999, 2000) has been used for the identification of immunodominant CD4+ epitopes within CMV pp65 protein. A matrix of 24 peptide pools, each containing 12 peptides (15-mers overlapping by 11 aa) (all 138 peptides) representing the whole pp65 protein was constructed. First, the peptide pools and later on the individual peptides have been checked for ability to provoke specific T cell response. The difference of this approach from others (Maeker, 2001; Tobery et al., 2001) is that it relies on the different techniques for monitoring T cell response, such as flow cytometric analysis (surface INF-gamma staining, CD4 and CD69 markers) and molecular methods (RT-PCR for TCR-Vβ content of CMV-specific CD4+ cells). The used technique, termed cytokine flow cytometry, allows detection of CMV-specific T-cells before either Ag induced proliferation or cell death, and therefore offers the possibility to determine the clonotypic content of CMV-specific T cells as it exists in vivo, unaltered by long-term in vitro culture. As a result, two pp65 epitopes (aa 489-503 and aa 509-523) contributing to presumably protective CMV-specific CD4+ response has been found in healthy CMV seropositive subjects.

Both, Tana et al. and Bitmansour et al., describe approaches which involve complex and thus only difficult to control biological, cellular assay, whereas the present invention represents a direct, biochemical isolation not comparable at all to the prior art, especially with respect to manageability and reproducibility: The use of “empty” MHC/HLA molecules according to the present invention makes all complex methods using binding events to MHC/HLA molecules being located on the surface of a living cell obsolete.

A method for isolating of T cell eptiopes according to the present invention wherein a pool of ligands is contacted with (“empty”) MHC/HLA molecules (and is not dependent on cellular systems) and then (preferably) the ligands which bind are tested in a T cell test with respect to their T cell activation capacity, is therefore a completely new approach, especially in comparison with the cell-dependent assays described. Also compared with “in silicio” methods, the present method provides “real world” utility with an easy and fast handling.

The performance and utility of the methods according to the present invention are demonstrated in the example section on a specific pathogen protein, pp65 of Cytomegalovirus (CMV). By this example, the effectiveness and advantageousness of the present invention are shown and compared to prior art methods.

CMV, a betaherpesvirus, is the major cause of non-Epstein-Barr virus (EBV) infectious mononucleosis in the general population and an important pathogen in the immunocompromised host, including patients infected with the human immunodeficiency virus (HIV) suffering from acquired immunodeficiency syndrome (AIDS), neonates and transplant recipients (Drew et al, 1999). Depending on the population surveyed, the prevalence of CMV seropositivity in various regions ranges from 40-100%. As with other herpesviruses, primary CMV infection is followed by a persistent infection; re- or super-infection also occurs under certain circumstances (Britt, 1999), however mostly without pathologic consequences in the immunocompetent host because of pre-existing immunity (Plotkin et al, 1999). CMV establishes slow, persistent infections in humans. It is controlled, but never eliminated in the immunocompetent host. Among others (for example active expression of immunosuppressive genes, which interfere with antigen processing and presentation) two factors seem to contribute to this constant escape from immunosurveillance: replication in immunopriviliged sites like salivary gland epithelia on the one hand and the formation of a latent reservoir in CD33+ monocytes. These macrophage precursors do not support replication of CMV, which is therefore arrested until cells differentiate into macrophages. Only after differentiation the cellular environment seems to become permissive for lytic infection. 90% of primary infections in immunocompetent individuals go clinically unrecognized (Nichols et al, 2000) and long-term immunity develops, which controls viral persistence and—albeit not being sterilizing—is protective.

A high risk of infection/reactivation exists for the following groups, who are highly jeopardized to develop CMV disease:
a) Fetuses from seronegative pregnant women
b) seronegative transplant patients, because it is difficult to find CMV negative grafts,
c) seropositive transplant and HIV patients, because their induced or acquired immunodeficiency allows for reactivation of CMV from latency.

Congenital CMV infection may lead to deafness and more important is as frequent a cause of mental retardation as the common genetic syndromes, trisomy 21 and fragile X chromosome. From the study of Fowler K B, Stagno S, Pass R F, et al. (Fowler et al, 1992) one can extrapolate, that about 9000 European and 8000 American infants (representing 1 out of 500 infants born in the United states) are harmed each year as a result of intrauterine CMV infection, of which only 10% are clinically apparent at birth. Although congenital CMV infection is largely silent at birth, its cumulative effect is large in terms of clinical sequelae and public health impact (Plotkin et al, 1999). The factor, which is most closely associated with poor outcome, is a primary maternal infection during gestation, which is not easily to be prevented in seronegative mothers, because the virus spreads easily by close person-to-person contact and is shed in high amounts by seropositive toddlers (Field et al, 1999).

In the immunocompromised adult, disease is most frequently seen in solid organ transplant (SOT) patients and allogeneic hematopoetic stem cell transplant (HCT) patients as well as in HIV infected individuals. In allogeneic HCT patients severe CMV disease still occurs in about 15% of the patients, with a mortality of about 50%. The risk factors associated with CMV disease in HCT are a CMV positive graft donor and an uninfected graft recipient, concomitant bacterial infections, fulminant hepatitis and graft-versus-host disease. For SOT such as those of kidney, liver, heart, lung or the pancreas, CMV disease is associated with decreased graft and patient survival. CMV causes a variety of infectious diseases syndromes itself. The consequences of CMV disease are similar in all transplant patients, although specific organ involvement frequently corresponds to the organ transplanted. In general, liver, pancreas, lung, intestinal, and heart transplant recipients have a greater incidence of CMV disease than kidney transplant recipients. Symptomatic infections occur in approximately 39-41% of heart-lung, 9-35% of heart, 22-29% of liver and pancreas, and 8-32% of renal transplant recipients not receiving antiviral prophylaxis. Moreover is CMV infection associated with an augmented immunosupressed state, which may explain the frequent opportunistic superinfections in transplant patients. Furthermore it seems to be involved in allograft dysfunction and indirectly in decreased patient survival as well as increased costs and longer hospital lengths of stay (Sia et al, 2000). To the latter three points CMV-s suspected involvement on the one hand in accelerated coronary arteriosclerosis found in patients with CMV infection after heart transplantation (Van Son et al, 1999 and Field et al, 1999) and on the other hand in the enhanced risk of Epstein-Barr Virus (EBV) related posttransplantation lymphoproliferative disease in CMV/EBV doublepositive transplant patients (Sia et al, 2000) might contribute. CMV retinits has been one of the most common disease manifestations in AIDS patients and has been reported to occur in about 30% of patients, threatening those patients with blindness. Recent evidence indicates that the loss of CD4+ lymphocytes correlates with the development of CMV disease in AIDS patients and therefore the introduction of HIV protease inhibitors and highly active antiretroviral therapy (HAART), the incidence of CMV retinitis has declined significantly (Field et al, 1999). However CMV disease can still occur within 4 months of HAART initiation despite adequate suppression of HIV replication. Furthermore antiretroviral therapy failure is a problem on the rise (Nichols et al, 2000).

In the last decade, considerable progress has been made in the use of antiviral chemotherapy to prevent and treat CMV infection. At present there are five drugs that have been approved in the US for the treatment of CMV: Ganciclovir (Cytovene, when used as intravenous and oral formulation or Vitrasert, when used as intravitreal implant formulation), Foscarnet (Foscavir), Cidofovir (Vistide) and Fomivirsen (Vitravene). The triphosphate equivalents of Ganciclovir and Cidofovir (requiring phosphorylation by viral and cellular enzymes in case of the former and cellular enzymes only in case of the latter) are competitive inhibitors of desoxyribonucleotidetriphosphates for the viral DNA polymerase, while the pyrophosphate analogue Foscarnet blocks the pyrophosphate-binding site of this enzyme. Fomivirsen is the first antisense designed oligonucleotide to gain FDA approval. However the mechanism of action of Fomivirsen has never been fully confirmed and may have many components besides antisense (Field et al, 1999). Because of inherent high toxicicities, high incidence of late CMV disease and high costs of universal prophylaxis, a preemptive strategy of antiviral therapy emerged that is based on the selective treatment of patients with a high risk for developing CMV disease based on early detection of reactivated CMV post-HCT (Zaia et al, 2000). In SOT patients prophylactic strategies are particularly attractive in the setting of a graft donor CMV carrier and an uninfected graft recipient. Valganciclovir, the valine ester of the active drug ganciclovir with markedly increased oral bioavailability, given for three months reduced the incidence of CMV disease in kidney transplant patients markedly. Moreover there was also a significant reduction in the proportion of patients with biopsy-proven rejection at six months after transplantation. However, long-term prophylaxis especially with available oral agents, which make this form of treatment more convenient for the patient than conventional intravenous formulation, may also increase the incidence of drug resistance (Nichols et al, 2000). Multidrug resistance to Ganciclovir, Cidofuvir and Foscarnet—all due to changes in the viral DNA polymerase—has already been observed (Field et al, 1999). Fomivirsen seems to be effective against such mutants (Nichols et al, 2000) but because of its probable antiviral activity besides its antisense mode of action (Field et al, 1999), Fomivirsen resistant strains seem likely to arise.

Therefore a focus was set on the (re)constitution of CMV specific immunity after transplantation, which might also prevent damages to babies of seronegative mothers. This is related to the question, how the immunocompetent host is controlling CMV infection, so as to learn, what is necessary to be restored in the non-immunocompetent host. Aggregate data argue that CMV antibodies are partly protective through reduction of viremia (dissemination of cell-free virus), but are little effective in HCT patients, where the virus persists cell-associated within the monocyte population, a reservoir, which cannot be destroyed by the use of antibodies (Plotkin et al, 1999). This argues strongly for the development of vaccines, which restore CD8+ and CD4+ T cell responses. The protective function of CMV specific alphabeta/T cells could definitively be established by adoptive transfer of these cells early after transplantation. In recipients of adoptively transferred CD8+ CMV-specific cytotoxic T lymphocytes (CTL) in a phase I study, cytolytic responses equivalent to those in the immunocompetent marrow donor were achieved immediately after the fourth infusion. However they declined over the ensuing several weeks in the subset of patients who failed to recover endogenous CD4+ CMV-specific T helper responses, which underlines the importance of generating both CD8+ and CD4+ T cell responses (Zaia et al, 2000). The potential importance of CD4+ T cells in CMV control is also suggested by the very high frequencies of specific CD4+ memory cells (about 2.0% of total CD4+ T cells) in normal CMV seropositive individuals (Waldrop et al, 1998). The close association between the degree of CD4+ T cell deficiency and CMV disease in HIV infection is also thought to be consistent with a crucial role for CD4+ T cells in control of CMV reactivation (Komanduri et al, 1998). Five general types of CMV-related vaccines have been described: attenuated live virus vaccines, recombinant live virus vaccines, DNA vaccines, whole protein vaccines, and peptide vaccines. An attenuated live virus vaccine, the “Towne strain”, was developed and tested in the 1970s both in renal transplant patients and women in childbearing years. Although this vaccine was shown to be immunogenic, the use of a live virus in the transplant population presents a potential risk. To a lesser extent this also holds true for recombinant poxviruses with limited potential for replication in humans (avipox). Approaches, that show promise in the animal model include introduction of DNA vectors encoding immunogenic proteins as a means to elicit CTL. Refinement of DNA vaccine technology, including the use of minimal cytotoxic and helper T cell epitopes as immunogens may result in even more efficient vaccines (Zaia et al, 2000).

Taken together the negative side effects and high costs of antiviral drugs, the limited application range/success of above mentioned approaches for a CMV vaccine and the good efficacy of adoptive transfer of T cell clones highlights the importance of finding new MHC class I and II restricted T cell epitopes. These epitopes should be presented by the most prevalent HLA molecules and therefore confer protection to the majority of the population irrespective of the individual HLA setting. CMV is one of the viruses with the highest protein-coding capacity known, with 170-200 open reading frames (Reddehase, 2000). Analysis of the T cell responses of asymptomatic donors, obviously controlling the virus successfully, however revealed the immunodominance of CMV pp65, pp150, IE-1 and gB (Zaia et al, 2000). This is probably due to the expression of several immunosuppressive genes, which interfere with the formation and egress of peptide loaded MHC class I and II molecules. Remarkably, the downregulation of MHC class I molecules seems to interfere little with recognition of CMV-infected cells by CMV pp65 or pp150-specific CTL. Very early after viral entry, during which these 2 structural proteins are delivered into the cytoplasm, this can be explained by the fact that these structural proteins can be processed and presented before the expression of immunosuppressive viral genes. CMV pp65 or pp150 specific CTL however, recognize infected cells also efficiently at later stages, when MHC levels are already reduced. This seems to be due to a host counter evasion strategy. Upon CMV infection a cellular gene is induced, which binds to a receptor on T cells and facilitates T cell activation even when the target cell expresses only a low peptide/MHC density (Zaia et al, 2000). On the other hand, there are indications, that pp65 interferes with the presentation of IE-1, the activator of all ensuing immunosuppressive geneproducts (Reddehase, 2000), which could explain the immunodominance of the pp65 antigen. Thus, T cells specific for pp65 are able to lyse virus infected cells at all stages of the replication cycle and may be essential for eliminating infected cells in vivo (Zaia et al, 2000).

According to a further aspect, the present invention also provides T cell epitopes identifyable by a method according to the present invention, said T cell epitopes being selected from the group consisting of polypeptides comprising the sequence KMQVIGDQYVK (SEQ ID NO:13), KMQVIGDQYV (SEQ ID NO:2), FTWPPWQAGI (SEQ ID NO:3), AMAGASTSA (SEQ ID NO:4), SDNEIHNPAV (SEQ ID NO:5), KYQEFFWDA (SEQ ID NO:6) or combinations thereof.

The present invention also provides HLA A0201 binding epitopes with T cell activating capacity identifyable by a method according to the present invention using HLA A0201 molecules as MHC/HLA molecules, said HLA A0201 binding epitopes being selected from the group consisting of polypeptides comprising the sequence RLLQTGIHV (SEQ ID NO:7), VIGDQYVKV (SEQ. ID NO:8), YLESFCEDV (SEQ ID NO:9) or combinations thereof. Although these sequences have been known to bind to HLA A0201, their useability to activate T cells is provided with the present invention.

The present invention further provides the use of a peptide comprising the sequence RPHERNGFTV (SEQ ID NO:10) for preparing a composition for activating T cells in an individual being B7-negative.

Additionally, the present invention provides the use of a peptide comprising the sequence DDVWTSGSDSDE (SEQ ID NO:11) for preparing a composition for activating T cells in an individual being B35-negative.

Moreover, the present invention also provides the use of a peptide comprising the sequence TPRVTGGGAM (SEQ ID NO:12) for preparing a composition for activating T cells in an individual being B7-negative.

Although these sequences have been described to have a specific HLA restriction (RPHERNGFTV (SEQ ID NO:10): B7, DDVWTSGSDSDE (SEQ ID NO:11): B35, TPRVTGGGAM (SEQ ID NO:12): B7), it was surprising that these sequences have a specificity being different from the known restriction (RPHERNGFTV (SEQ ID NO:10): non-B7, DDVWTSGSDSDE (SEQ ID NO:11): non-B35, TPRVTGGGAM (SEQ ID NO:12): non-B7). This enables an expansion of the usefulness of these sequences, e.g. by making these sequences available as suitable vaccines for individuals expressing other HLAs than the ones described.

The present invention further provides peptides binding to class II HLA molecules selected from peptide nos. 55-64, 109, 383, 384, 421, 449-454, 469 and 470 according to table 3 of the example section.

Preferably, the epitopes or peptides according to the present invention further comprises 1 to 30, preferably 2 to 10, especially 2 to 6, naturally occurring amino acid residues at the N-terminus, the C-terminus or at the N- and C-terminus. For the purposes of the present invention the term “naturally occurring” amino acid residue relates to amino acid residues which are present in the naturally occurring protein at the specific position, relative to the epitope or peptide. For example, for the “AMAGASTSA” (SEQ ID NO:4) epitope, the naturally occurring amino acid residue at the N-terminus is Gly; the three naturally occurring amino acid residues at the C-terminus are Gly-Arg-Lys. A “non-naturally occurring” amino acid residue is therefore any amino acid residue being different as the amino acid residue at the specific position relative to the epitope or peptide.

According to a preferred embodiment of the present invention, the present epitopes or peptides further comprise non-naturally occurring amino acid(s), preferably 1 to 1000, more preferred 2 to 100, especially 2 to 20 non-naturally occurring amino acid residues, especially at the N-terminus, the C-terminus or at the N- and C-terminus. Also combinations of non-naturally and naturally occurring amino acid residues are possible under this specific preferred embodiment. The present epitope may also contain modified amino acids (i.e. amino acid residues being different from the 20 “classical” amino acids, such as D-amino acids or S-S bindings of Cys) as additional amino acid residues or in replacement of a naturally occuring amino acid residue.

It is clear that also epitopes or peptides derived from the present epitopes or peptides by amino acid exchanges improving, conserving or at least not significantly impeding the T cell activating capability of the epitopes are covered by the epitopes or peptides according to the present invention. Therefore, the present epitopes or peptides also cover epitopes or peptides, which do not contain the original sequence as derived from CMV pp65, but trigger the same or preferably an improved T cell response. These epitopes are referred to as “heteroclitic”. These include any epitope, which can trigger the same T cells as the original epitope and has preferably a more potent activation capacity of T cells preferably in vivo or also in vitro.

Heteroclitic epitopes can be obtained by rational design i.e. taking into account the contribution of individual residues to binding to MHC/HLA as for instance described by Ramensee et al. 1999 or Sturniolo et al. 1999, combined with a systematic exchange of residues potentially interacting with the TCR and testing the resulting sequences with T cells directed against the original epitope. Such a design is possible for a skilled man in the art without much experimentation.

Another possibility includes the screening of peptide libraries with T cells directed against the original epitope. A preferred way is the positional scanning of synthetic peptide libraries. Such approaches have been described in detail for instance by Blake et al 1996 and Hemmer et al. 1999 and the references given therein.

As an alternative to epitopes represented by the cognate CMV pp65 derived amino acid sequence or heteroclitic epitopes, also substances mimicking these epitopes e.g. “peptidemimetica” or “retro-inverso-peptides” can be applied.

Another aspect of the design of improved epitopes is their formulation or modification with substances increasing their capacity to stimulate T cells. These include T helper cell epitopes, lipids or liposomes or preferred modifications as described in WO 01/78767.

Another way to increase the T cell stimulating capacity of epitopes is their formulation with immune stimulating substances for instance cytokines or chemokines like interleukin-2, -7, -12, -18, class I and II interferons (IFN), especially IFN-γ, GM-CSF, TNF-alpha, flt3-ligand and others.

According to a further aspect, the present invention is drawn to the use of an epitope or peptide according to the present invention for the preparation of a HLA restricted vaccine for treating or preventing cytomegalovirus (CMV) infections.

The invention also encompasses the use of an epitope comprising the sequence KMQVIGDQYV (SEQ ID NO:2), FTWPPWQAGI (SEQ ID NO:3), AMAGASTSA (SEQ ID NO:4), SDNEIHNPAV (SEQ ID NO:5) and/or KYQEFFWDA (SEQ ID NO:6) for the preparation of a vaccine for treating or preventing cytomegalovirus (CMV) infections.

Consequently, the present invention also encompasses a vaccine for treating or preventing cytomegalovirus (CMV) infections comprising an epitope comprising the sequence KMQVIGDQYV (SEQ ID NO:2), FTWPPWQAGI (SEQ ID NO:3), AMAGASTSA (SEQ ID NO:4), SDNEIHNPAV (SEQ ID NO:5) and/or KYQEFFWDA (SEQ ID NO:6). Furthermore, also a HLA specific vaccine for treating or preventing cytomegalovirus (CMV) infections comprising the epitopes or peptides according to the present invention is an aspect of the present invention. The application of the corresponding nucleic acids encoding the peptides according to the present invention, e.g. as DNA vaccine, also falls under the scope of the present invention, at least as equivalent to the claimed peptide vaccines.

Parker et al. (1994) present the method of prediction of peptide binding to MHC class I molecules based on the experimental data of binding of individual peptides. The peptide binding ability was assessed indirectly by monitoring the ability of peptides to promote incorporation of beta2-microglobulin (β2m) into HLA-A2/β2 m/peptide heterodimeric complexes. The experimental data (measured rates of dissociation of β2m) allows to create the value of corresponding coefficients used then to calculated theoretical binding stability for any nonapeptides in the complex with HLA-A2 molecules. In this study it has been shown for the first time that CMV pp65 derived peptide sequence RLLQTGIHV (SEQ ID NO:7) can stabilize HLA-A2/β32 m/peptide complex.

Morgan et al. (1998) describe the method to determine the binding affinity of peptides to purified HLA molecules also based on the indirect measurement of the incorporation of β2m into the HLA/peptide complex. In this study the in vitro binding of RLLQTGIHV (SEQ ID NO:7) peptide has been demonstrated and its relative binding affinity to HLA-A2 molecule (association and dissociation constants) was measured.

Although binding to HLA-A2 is demonstrated by Parker et al. and Morgan et al., the final proof that this ligand also represents a true T-cell epitope is lacking, whereas in the present invention this is demonstrated by means of interferon-gamma ELIspot proofing, that RLLQTGIHV (SEQ ID NO:7) can not only bind to HLA-A2, but that it can only induce functional T-cells. This is not a trivial finding: it is well known in the field that many ligands binding with high affinity do not represent T-cell targets, as they are for instance not or not efficiently generated through the afore mentioned “antigen processing and presentation pathway. Therefore, HLA A0201 binding activity of RLLQTGIHV (SEQ ID NO:7) was not only surprising but may also be selectively used in vaccines specifically designed e.g. for a certain allele population.

Preferably, such a vaccine according to the present invention further comprises an immunomodulating substance, preferably selected from the group consisting of polycationic substances, especially polycationic polypeptides, immunomodulating nucleic acids, especially deoxyinosine and/or deoxyuracile containing oligodeoxynucleotides, or mixtures thereof.

Preferably the vaccine further comprises a polycationic polymer, preferably a polycationic peptide, especially polyarginine, polylysine or an antimicrobial peptide.

The polycationic compound(s) to be used according to the present invention may be any polycationic compound which shows the characteristic effect according to the WO 97/30721. Preferred polycationic compounds are selected from basic polypeptides, organic polycations, basic polyaminoacids or mixtures thereof. These polyaminoacids should have a chain length of at least 4 amino acid residues. Especially preferred are substances containing peptidic bounds, like polylysine, polyarginine and polypeptides containing more than 20%, especially more than 50% of basic amino acids in a range of more than 8, especially more than 20, amino acid residues or mixtures thereof. Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be polycationic anti-bacterial microbial peptides. These (poly)peptides may be of prokaryotic or animal or plant origin or may be produced chemically or recombinantly. Peptides may also belong to the class of defensines. Such host defense peptides or defensines are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substance in the present invention are cathelicidin derived antimicrobial peptides or derivatives thereof (A 1416/2000, incorporated herein by reference), especially antimicrobial peptides derived from mammal cathelicidin, preferably from human, bovine or mouse, or neuroactive compounds, such as (human) growth hormone (as described e.g. in WO01/24822).

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin, especially mouse, bovine or especially human cathelins and/or cathelicidins. Related or derived cathelin substances contain the whole or parts of the cathelin sequence with at least 15-20 amino acid residues. Derivations may include the substitution or modification of the natural amino acids by amino acids which are not among the 20 standard amino acids. Moreover, further cationic residues may be introduced into such cathelin molecules. These cathelin molecules are preferred to be combined with the antigen/vaccine composition according to the present invention. However, these cathelin molecules surprisingly have turned out to be also effective as an adjuvant for a antigen without the addition of further adjuvants. It is therefore possible to use such cathelin molecules as efficient adjuvants in vaccine formulations with or without further immunactivating substances.

Another preferred polycationic substance to be used according to the present invention is a synthetic peptide containing at least 2 KLK-motifs separated by a linker of 3 to 7 hydrophobic amino acids, especially L (e.g. KLKL₅KLK; PCT/EP01/12041, incorporated herein by reference).

The immunomodulating (or:immunogenic) nucleic acids to be used according to the present invention can be of synthetic, prokaryotic and eukaryotic origin. In the case of eukaryotic origin, DNA should be derived from, based on the phylogenetic tree, less developed species (e.g. insects, but also others). In a preferred embodiment of the invention the immunogenic oligodeoxynucleotide (ODN) is a synthetically produced DNA-molecule or mixtures of such molecules. Derivates or modifications of ODNs such as thiophosphate substituted analogues (thiophosphate residues substitute for phosphate) as for example described in U.S. Pat. No. 5,723,335 and U.S. Pat. No. 5,663,153, and other derivatives and modifications, which preferably stabilize the immunostimulatory composition(s) but do not change their immunological properties, are also included. A preferred sequence motif is a six base DNA motif containing an (unmethylated) CpG dinucleotide flanked by two 5′ purines and two 3′ pyrimidines (5′-Pur-Pur-C-G-Pyr-Pyr-3′). The CpG motifs contained in the ODNs according to the present invention are more common in microbial than higher vertebrate DNA and display differences in the pattern of methylation. Surprisingly, sequences stimulating mouse APCs are not very efficient for human cells. Preferred palindromic or non-palindromic ODNs to be used according to the present invention are disclosed e.g. in Austrian Patent applications A 1973/2000, A 805/2001, EP 0 468 520 A2, WO 96/02555, WO 98/16247, WO 98/18810, WO 98/37919, WO 98/40100, WO 98/52581, WO 98/52962, WO 99/51259 and WO 99/56755 all incorporated herein by reference. Apart from stimulating the immune system certain ODNs are neutralizing some immune responses. These sequences are also included in the current invention, for example for applications for the treatment of autoimmune diseases. ODNs/DNAs may be produced chemically or recombinantly or may be derived from natural sources. Preferred natural sources are insects.

Alternatively, also nucleic acids based on inosine and cytidine (as e.g. described in the PCT/EP01/06437) or deoxynucleic acids containing deoxy-inosine and/or deoxyuridine residues (described in the Austrian patent applications A 1973/2000 and A 805/2001, incorporated herein by reference) may preferably be used as immunostimulatory nucleic acids for the present invention.

Of course, also mixtures of different immunogenic nucleic acids may be used according to the present invention.

Preferably, the present vaccine further comprises a pharmaceutically acceptable carrier.

According to a further preferred embodiment, the present vaccine comprises an epitope or peptide which is provided in a form selected from peptides, peptide analogues, proteins, naked DNA, RNA, viral vectors, virus-like particles, recombinant/chimeric viruses, recombinant bacteria or dendritic cells pulsed with protein/peptide/RNA or transfected with DNA comprising the epitopes or peptides.

According to a further aspect, the present invention is drawn to T cells, a T cell clone or a population (preparation) of T cells specifically recognizing any epitope or peptide according to the present invention, especially a CMV epitope as described above. A preferred application of such T cells is their expansion in vitro and use for therapy of patients e.g. by adoptive transfer. Therefore, the present invention also provides the use of T cells, a T cell clone or a population (preparation) of T cells for the preparation of a composition for the therapy of CMV patients.

Such T cells (clones or lines) according to the present invention, specifically those recognizing the aforementioned CMV peptides are also useful for identification of heteroclitic epitopes, which are distinct from the originally identified epitopes but trigger the same T cells.

Such cells, compositions or vaccines according to the present invention are administered to the individuals in an effective amount.

### BRIEF DESCRIPTION OF THE DRAWINGS

The invention will be explained in more detail by way of the following examples and drawing figures, to which, however it is not limited.

FIG. 1 shows peptide binding affinities to soluble DR4 molecules;

FIG. 2 shows identification of peptides capable of binding to empty DR4 molecules (A. Purification of HLA-peptide complexes; B. MS analysis of bound peptides);

FIG. 3 shows the binding of high affinity peptide in the presence of the excess of low affinity peptide to DR4 molecules;

FIG. 4 shows the binding of the individual peptides and peptide mixtures to DR4 molecules;

FIG. 5 shows an Elispot with (5a) isolated CD4+ T cells and DR0401 molecules (costimulation with anti CD28) and with (5b) isolated CD8+ T cells and HLA A0201 molecules (costimulation with anti CD28 mab);

FIG. 6 shows the CMV pp65 peptide pool array;

FIG. 7 shows (7a) the binding of peptide pools derived from pp65 protein to DR4 molecules and (7b) binding of single pp65 peptides to DR4 molecules; in FIG. 7b, peptide P54=SEQ ID NO:79; P55=SEQ ID NO:80; P56=SEQ ID NO:81; P57=SEQ ID NO:82; P58=SEQ ID NO:83; P59=SEQ ID NO:84; P60=SEQ ID NO:85; P61=SEQ ID NO:86; P62=SEQ ID NO:87; P63=SEQ ID NO:88; P174=SEQ ID NO:199; P175=SEQ ID NO:200; P176=SEQ ID NO:201; P177=SEQ ID NO:202; P178=SEQ ID NO:203; P179=SEQ ID NO:204; P180=SEQ ID NO:205; P181=SEQ ID NO:206; P357=SEQ ID NO:382; P358=SEQ ID NO:383; P359=SEQ ID NO:384; P360=SEQ ID NO:385; P361=SEQ ID NO:386; P362=SEQ ID NO:387; P380=SEQ ID NO:405; P381=SEQ ID NO:406; P382=SEQ ID NO:407; P383=SEQ ID NO:408; P384=SEQ ID NO:409; P385=SEQ ID NO:410; P507=SEQ ID NO:532; P508=SEQ ID NO:533; P509=SEQ ID NO:534; P510=SEQ ID NO:535;

FIG. 8 shows the 1^{st} screen with peptide mixtures (containing 21 peptides each, Donor #10736 HLA A2/3, HLA B15/35; 8a) and the 1^{st} screen with peptide mixtures (containing 21 peptides each, Donor #10687 HLA A2/11, HLA B7/13; 8b);

FIG. 9 shows the 2^{nd} screen with single peptides (Donor #10736 HLA A2/3, HLA B15/35);

FIG. 10 shows PBMC from subject 10788 applied for IFN-γ ELIspot with CMVpp65 15mers 57, 59 and controls (med: no peptide, HIV: irrelevant HIV-derived peptide, ConA: polyclonal stimulation;

FIG. 11 shows confirmation of simultaneous CD4+ and CD8+ T-cell responses against CMVpp65 15mers 469, 470 by intracellular IFN-γ staining; and

FIG. 12 shows mapping of DRB1*0401 epitopes with overlapping 15 mers in transgenic mice using IFN-γ ELISpot assay: One week later after the last vaccination, spleens were removed and cells were activated ex vivo with relevant peptides (no. 1500-1505), overlapping 15mers representing these longer peptides and irrelevant influenza hemagglutinin derived peptide (no. 1171) to determine IFN-γ-producing specific cells (medium control is subtracted).

### DETAILED DESCRIPTION OF ILLUSTRATIVE EMBODIMENTS

### EXAMPLES

General Description of the Examples:

The present examples show the performance of the present invention on a specific pathogen protein, pp65 of CMV.

In the first part the method according to the present invention was applied, which is based on the use of “empty HLA molecules”. These molecules were incubated with mixtures of potential CMV derived peptide ligands, screening for specific binding events. The complexes formed in this way were isolated and used for the identification of the specifically bound ligands. The possibility to use highly complex mixtures allows a very quick identification of the few binders out of hundreds or even thousands of potential ligands. This is demonstrated by using HLA-DRB1*0401 and pools of overlapping 15-mers.

However, the same process can be applied for class I molecules and peptides of appropriate length i.e. 8 to 11-mers. The ligand-pools can be synthetic overlapping peptides. Another possibility is to digest the antigen in question enzymatically or non-enzymatically. The latter achieved by alkali-hydrolysis generates all potential degradation products and has been successfully used to identify T cell epitopes (Gavin 1993). Enzymatic digestions can be done with proteases. One rational way would further be to use proteases involved in the natural antigen-processing pathway like the proteasome for class I restricted epitopes (Heemels 1995) or cathepsins for class II restricted epitopes (Villadangos 2000). Ligand pools could also be composed of naturally occurring ligands obtained for instance by lysis of or elution from cells carrying the respective epitope. In this regard it is important to note that also non-peptide ligands like for instance glycolipids can be applied. It is known that nonclassical class I molecules, which can be encoded by the MHC (e.g. HLA-G, HLA-E, MICA, MICB) or outside the MHC (e.g. CD1 family) can present various non-peptide ligands to lymphocytes (Kronenberg 1999). Use of recombinant “empty” nonclassical class I molecules would allow binding reactions and identification of binders in similar manner as described here.

After rapid identification of ligands capable of binding to HLA molecules the process according to the present invention also offers ways to characterize directly specific T cell responses against these binders. One possibility is to directly use the isolated HLA:ligand complex in a so called “synthetic T cell assay”. The latter involves antigen-specific re-stimulation of T cells by the HLA:ligand complex together with a second signal providing co-stimulation like activation of CD28 by an activating antibody. This assay can be done in an ELIspot readout as demonstrated in Example II.

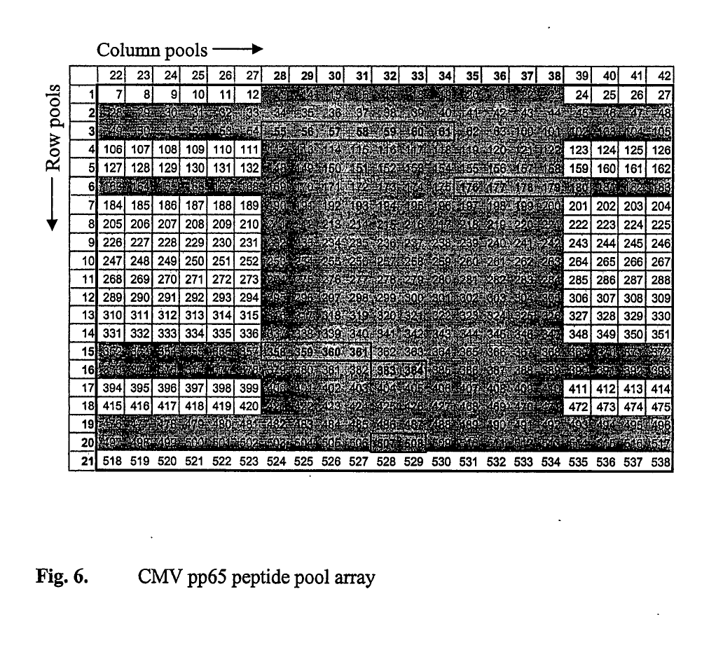

Here, it was chosen to synthesize the CMV pp65 as a series of overlapping 15 mer peptides, each peptide overlapping its precursor by 14 out of 15 aa. The peptides were supplied as pools of 21 single peptides, which were constructed that way that each single peptide occurs in exactly 2 pools. Array of the peptide pools in matrix form allows identification of single peptides as the crossover points of row- and column mixtures (FIG. 6). As donors 10 healthy CMV seropositive blood donors, expressing the MHC class I molecule A2, were chosen. This MHC preference was inferred because it is known, that the pp65 antigen is especially well recognized by donors carrying this MHC molecule (Saulquin et al, 2000; Kern et al, 2000).

In parallel the same peptides (pools) were screened by interferon-gamma (IFN-□) ELIspot assays using peripheral blood mononuclear cells (PBMC) from CMV seropositive individuals (binding to soluble recombinant HLA-class II molecules in an SDS-stability assay). Both approaches yielded epitopes, and although not biased by the choice of HLA (class I HLA-A2 for the ex vivo T cell assay approach, class II HLA-DR4 for the peptide binding approach) showed some degree of overlap.

Materials & Methods

Peptides

547 fifteen amino acid residue (15mer) peptides overlapping by 14 aa representing the complete sequence of the CMV pp65 antigen, were synthesized on a Syro II synthesizer (Multisyntech, Witten, Germany). 288 peptides were made in parallel using standard F-moc chemistry.

Each peptide was solubilized in 100% DMSO at ˜10 mg/ml. Stocks of 42 peptide pools derived from pp65 were made in 100% DMSO at a final concentration of 0.45 mg/ml for each peptide.

The other peptides were synthesized using standard F-moc chemistry either on the Syro II synthesizer or a ABI 433A synthesizer (Applied Biosystems, Weiterstadt, Germany) and purified by RP-HPLC (Biocut 700E, Applied Biosystems, Langen, Germany) using a C18 column (either ODS ACU from YMC or 218TP from Vydac). Purity and identity of each peptide were characterized by MALDI-TOF on a Reflex III mass-spektrometer (Bruker, Bremen, Germany).

Peptide Binding Assay

Soluble HLA class I A*0201 and HLA class II DRA1*0101/DRB1*0101/Ii, DRA1*0101/DRB1*0401/Ii and DRA1*0101/DRB1*0404/Ii molecules were expressed in SC-2 cells and purified as described in Aichinger et al., 1997.

In peptide binding reactions HLA molecules were used in a concentration of 0.5 μM, and each single peptide was added in 10-fold molar excess (5 μM) if not mentioned differently. The concentration of DMSO in the binding reaction did not exceed 4%. The reaction was performed in PBS buffer (pH 7.4) at room temperature for 48 hours in the presence of a protease inhibitor cocktail (Roche) and 0.1% octyl-b-D-glucopyranoside (Sigma).

Peptide binding was evaluated in an SDS-stability assay (Gorga et al., 1987): trimeric HLA class II ab-peptide complexes are resistant to SDS and consequently appear as ˜60 kDa band in SDS-PAGE Western blot analysis. Individual HLA class II α and β chains not stabilized by a peptide binding with intermediate to high affinity migrate as ˜35 kDa and ˜30 kDa bands, respectively.

Briefly, HLA-peptide complexes were treated with 1% SDS at room temperature and resolved by SDS-PAGE run with 20 mA for approximately 2.5 hours at room temperature. Protein was transferred onto PVDF membrane by electroblotting, and stained with anti-a-chain TAL.1B5 α or/and β-chain MEM136 antibodies. For detection of Western-blot signals ECL solutions (Amersham) were used.

The binding affinities to DRB1*0701 and DRB1*1101 were tested by a peptide-competition assay (Reay et al., 1992). Briefly, binding of the biotinylated CLIP peptide (reference peptide) has been used for monitoring of HLA:peptide complex formation. A testing peptide added to the binding reaction at an equimolar concentration to CLIP peptide could compete out CLIP when its affinity is higher or inhibit binding for 50% if its affinity is equal to affinity of CLIP. In the case of lower affinity peptides they should be added in excess to the reference peptide to compete for occupancy of HLA binding grove. The values of the concentration of competitor peptides required for 50% inhibition of reference peptide binding (IC₅₀) can be used for evaluation of peptide binding affinities. Alternatively, comparing of the amount of reference peptide bound to HLA molecules in the presence or absence of competitor peptide one can determine the binding activity of the peptide of interest.

In the present peptide-competition assay conditions of peptide binding were similar to described above. Soluble HLA molecules were used in the concentration of 0.5 μM and biotinylated CLIP was added to all samples in the final concentration of 2 μM. Competitor peptides were added in three different concentrations: 0.25, 5 and 100 μM. Binding reaction was performed in PBS buffer (pH 7.4) for 18 hours at 37° C. Amount of biotinylated CLIPm, associated with soluble HLA molecules, was determined by ELISA. Briefly, MaxiSorb 96-well plates (Nunc, Denmark) were coated with mouse anti-αβ antibody L243 (purified from ATCC HB-55) by overnight incubation with 50 μl of 10 μg/ml dilution in PBS at 4° C. Non-specific binding to the plastic was blocked by incubation with T-PBS containing 3% of BSA for 2 hours at 37° C. and binding reactions were then “captured” for 2 hours at room temperature. After extensive washing of the plates with T-PBS, HLA-assosiated peptide complexes were detected colorimetricaly using alkaline phosphatase-streptavidin conjugate (Dako) and Sigma 104 phosphatase substrate (Sigma Diagnostics, USA). The optical density at 405 nm was measured on a microplate reader SUNRISE (Tecan).

Identification of Peptides from HLA-Peptide Complexes

After peptide binding reaction with soluble HLA molecules, HLA-peptide complexes were separated from free peptides by gel-filtration chromatography on Superdex-200 column (AKTAdesign, Amersham Pharmacia Biotech). HLA-containing fractions were collected, and bound peptides were reconstituted from the complexes by adding TFA to the final concentration of 1%. Peptides were desolted by Ziptip purification and analyzed by MALDI-TOF mass-spectrometry.

Synthetic Elispot Assays

96 well filtration plates from Millipore (catalog-number: MAHA S4510) were coated with a mixture of 1 μg/well anti human IFN-γ mab B140 from Bender Med Systems and 0.5 μg/well QIAexpress Penta. His antibody from Qiagen over night at 4° C. As a positive control for viability and cytokine production of CD8+ T cells some wells were coated with anti CD3 antibody, clone MEM-57 from V. Horejsi, Institute of Molecular Genetics, Prag.

Plates were washed 2 times with PBS (from GIBCOBRL, catalognumber 14190-094) and blocked with the following ELISPOT medium: RPMI 1640 from GIBCOBRL (catalog number: 31870-025) supplemented with 1 mM sodium pyruvate from GIBCOBRL (catalog number: 11360-039), 2 mM L-glutamine from GIBCOBRL (catalog number: 25030-024), 0.1 mM non-essential amino acids from GIBCOBRL (catalog number: 11140-035), 50 μg/ml gentamycin from GIBCOBRL (catalog number: 15710-049), 50 μM 2-mercaptoethanol from GIBCOBRL (catalog number: 31350-010) and 10% human serum type AB from BioWhittaker-(catalog number: 14-490E) for 30 min a 37° C.

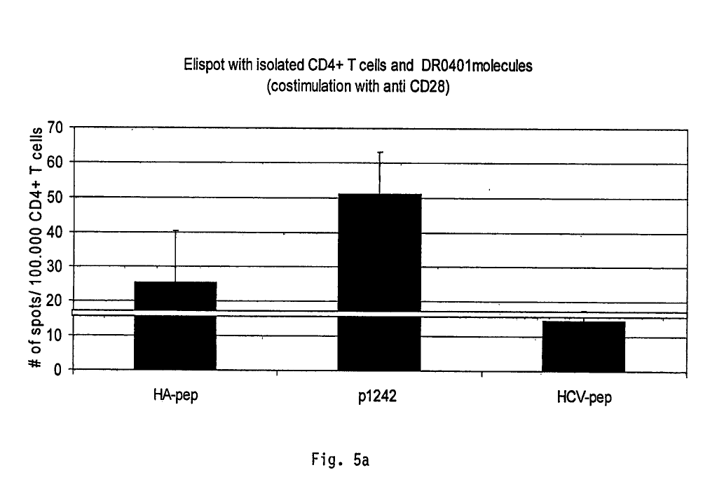

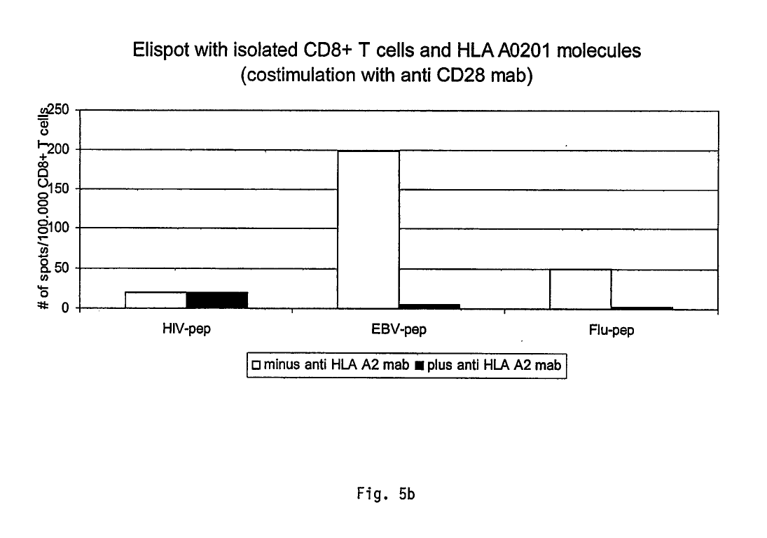

After removal of the blocking medium wells were incubated with soluble HLA DRB1*0401 loaded with either peptide 1242 derived from M. tuberculosis, peptide 1171 derived from Influenza Hemagglutinin (HA-pep: aa 306-318 or for a negative control peptide 84 derived from Hepatitis C virus (HCV-pep: NS3 aa1248-1261), diluted in ELISPOT medium to 100 μg/ml (10 μg/well), for 5 hours at 37° C. (FIG. 5a). For FIG. 5b soluble HLA A*0201 molecules loaded either with a peptide derived from the EBV antigen BMLF1 aa280-88, peptide 21 derived from Influenza Matrix protein aa 58-66 or for a negative control peptide 90 derived from HIV reverse transcriptase aa 476-484 were used. Loading of molecules was essentially done as described in the paragraph “Peptide binding assay”.

Mononuclear cells from the peripheral blood (PBMC) of healthy, BCG-vaccinated HIV and HCV negative donors with matching HLA phenotype were isolated on Lymphoprep (from Nycomed Pharma AS, Oslo, Norway) using Leuco Sep tubes (from Greiner), washed 3× with PBS (from GIBCOBRL, catalognumber 14190-094).

From the PBMC either the CD4+ T cells (FIG. 5a) or the CD8+ T cells (FIG. 5b) were isolated using MACS technique (Miltenyi, Germany) according to the manufacturer's instructions. The isolated T cells were resuspended in ELISPOT medium containing 10 μg/ml anti CD28 monoclonal antibody (clone 37407.111, mab 342 from R&D systems) at a concentration of 1 Mio/ml.

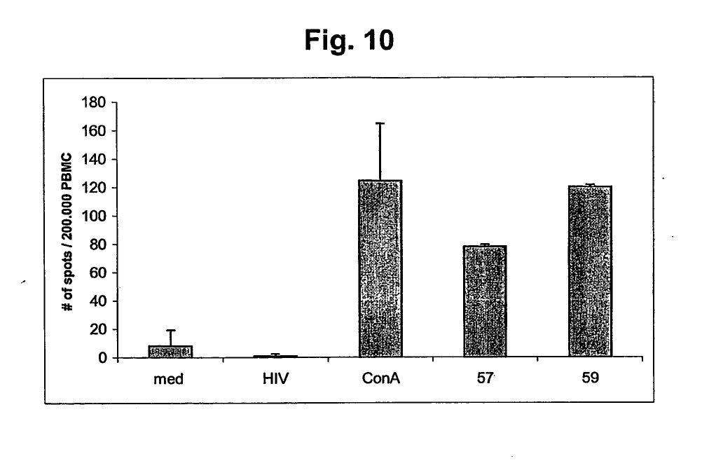

The solution containing the soluble HLA (sHLA) molecules was discarded and the isolated T cells were seeded into the wells. The respective samples were resupplemented with the according peptides at 5 μM (FIG. 5a) or 10 μg/ml (FIG. 5b).

Cells were cocultivated with the sHLA molecules for 20 hrs.

Assays were arrested by shaking off the contents and washing 6× with wash buffer (PBS; 0.1% Tween 20 from SIGMA). Next 100 μl of a 1:10000 dilution of the biotinylated anti human IFN-γ mab (B308-BT2 from BMS), which corresponds to 0.015 μg/well, was added for an incubation of 2 hrs at 37° C. or alternatively for over night at 4° C. After washing, Streptavidin-ALP from DAKO (catalog number: D0396) was added at 1.2 μg/ml for 1 hr at 37° C. The assay was developed by addition of 100 μl/well BCIP/NBT alkaline phosphatase substrate from SIGMA (catalog number: B-5655).

Spots were counted on an Elispot reader (Bioreader 2000 from Biosys, Germany) in the size range between a diameter of 130 μm to 2000 μm. The average number of spontaneously induced IFN-γ spots was deduced from negative control samples (HCV or HIV peptide). Every response excelling the average spontaneous IFN-γ release, to which value 2× the standard deviation of the negative control samples was added, was considered significant.

Collection, Preparation and Cryopreservation of PBMC for Screening T Cells Responses Against the CMV pp65 Antigen

PBMC of 9 CMV seropositive HLA A2 healthy volunteers were included in this screen. HLA information of these donors on HLA class I A and B was available, but not on HLA class I C nor on HLA class II.

Whole blood was collected in ACD Vacutainer tubes (Becton Dickinson, Europe). PBMC were separated from whole blood on Lymphoprep (from Nycomed Pharma AS, Oslo, Norway) using Leuco Sep tubes (from Greiner), washed 3× with PBS (from GIBCOBRL, catalognumber 14190-094) and resuspended at a concentration of 20 Mio c/ml in the following freezing medium: 4 parts RPMI 1640 from GIBCOBRL (catalog number: 31870-025) supplemented with 1 mM sodium pyruvate from GIBCOBRL (catalog number: 11360-039), 2 mM L-glutamine from GIBCOBRL (catalog number: 25030-024), 0.1 mM non-essential amino acids from GIBCOBRL (catalog number: 11140-035), 50 μg/ml gentamycin from GIBCOBRL (catalog number: 15710-049), 50 μM 2-mercaptoethanol from GIBCOBRL (catalog number: 31350-010); 5 parts fetal calf serum (FCS) from PAA (catalog number: A11-042); prior to use 1 part DMSO from SIGMA cell culture (catalog number D2650) was added.

PBMC were stored over night in freezing containers (Nalgene 1° C. freezing container, catalog number 5100-0001) at −80° C. and then transferred into the liquid nitrogen container.

ELISPOT Assay for single cell human IFN-gamma release: Detection of pp65-specific effectors from frozen PBMC

The assay was essentially done as described in Lalvani et al. Briefly, Multi Screen 96 well filtration plates from Millipore (catalog-number:MAHA S4510) were coated with 10 μg/ml anti human IFN-γ mab B140 from Bender Med Systems (1 μg/well) over night at 4° C. Plates were washed 2 times with PBS (from GIBCOBRL, catalognumber 14190-094) and blocked with the following ELISPOT medium: RPMI 1640 from GIBCOBRL (catalog number: 31870-025) supplemented with 1 mM sodium pyruvate from GIBCOBRL (catalog number: 11360-039), 2 mM L-glutamine from GIBCOBRL (catalog number: 25030-024), 0.1 mM non-essential amino acids from GIBCOBRL (catalog number: 11140-035), 50 μg/ml gentamycin from GIBCOBRL (catalog number: 15710-049), 50 μM 2-mercaptoethanol from GIBCOBRL (catalog number: 31350-010) and 10% human serum type AB from BioWhittaker (catalog number: 14-490E).

Cryopreserved PBMC were thawed quickly in a 37° C. water bath and washed 2 with ELISPOT medium. Following thawing, PBMC were placed for 2 h into an incubator (37° C., 5% CO₂). After this incubation, cells were filtered through a 70 μm cell strainer (Falcon), adjusted to a concentration of 2 Mio/ml and plated at 200.000 PBMC/well.

PBMC were cocultivated with either peptide pools, where each single peptide was contained at a final concentration of 5 μg/ml, or individual peptides at a final concentration of 10 μg/ml for 20 hrs. As an assay control polyclonal induction of IFN-γ secretion by Concanavalin A (SIGMA) was used. Spontaneous IFN-γ release was measured by either incubating PBMC with medium alone or—since exclusively HIV-negative donors were screened—by addition of an HLA 0201 restricted CTL epitope from HIV (HIV Reverse Transcriptase, aa 476-484: ILKEPVHGV (SEQ ID NO:574)). As positive control peptides frequently recognized HLA A 0201 restricted CTL epitopes from Influenza Matrixprotein (aa58-65: GILGFVFTL) (SEQ ID NO:14) and EBV BMLF1 antigen (aa 280-288: GLCTLVAML)(SEQ ID NO:15) were included in the screen.

Assays were developed as described in the paragraph “Synthetic Elispot assays”.

Immunization of HLA-Transgenic Mice

In the first set of experiments for immunogenicity the longer synthetic peptides incorporating candidate epitope sequences were injected into HLA-DRB1*0401-transgenic mice as follows: groups of 3 mice (female mice, 8 weeks of age) were injected subcutaneous into the hind footpad (in total 300 μg of peptide and 5 nanomole of CpG1668 per mouse) once.

In the second set of experiments for epitope mapping synthetic CMV-derived peptides were tested in HLA-DRB1*0401-transgenic mice as follows: groups of 6 mice (female mice, 8 weeks of age) were injected subcutaneous into the flank (in total 100 μg of peptide and 50 μl of CFA once and twice with 50 μl of IFA per mouse) 3 times in weekly intervals.

In the third set of experiments for epitope mapping synthetic CMV-derived peptides were tested in HLA-DRB1*0401-transgenic mice as follows: groups of 8 mice (female mice, 8 weeks of age) were injected subcutaneous into the hind footpad (in total 300 μg of peptide and 5 nanomole of CpG1668 per mouse for peptide no. 1500 and the same amount of peptide no. 1503 and no. 1504 with 50 μl of IFA per mouse) once.

Isolation of Murine Splenocytes and Separation of CD4+/CD8+ T Cells

Spleens were removed on day 7 after last injection and pooled together for each group. To prepare the single cell suspension spleens were smashed in DMEM medium supplemented with 5% FCS 2 mmole L-glutamine, 50 μg-ml gentamycine, 1% sodium pyruvate, 0.1% 2-mercaptoethanol and 1% nonessential amino acids (PAA Laboratories, Linz, Austria) (complete medium), and filtered through a 70 μm cell strainer. Cells were washed in complete medium (1200 rpm, 10 minutes), the pellet was resuspended in red blood cell lysing buffer (Sigma-Aldrich) and incubated for 2 minutes to remove erythrocytes. After washing, cells were counted using KOVA Glasstic slides (Hycor, Biomedical INC.) and adjusted to the concentration 1×10⁷; 3.3×10⁶ and 1.1×10⁶ cells per ml with complete medium.

To determine class I or class II restriction of potential epitopes, total cells from murine spleens were separated into CD4+ and CD8+ populations by using MACS separation system with miniMACS and midiMACS columns and anti-CD4 and anti-CD8 magnetic beads (Miltenyi Biotec, Germany), according to supplier recommended procedure. The purity of CD4+ and CD8+ populations was checked by FACS analysis after staining shortly described as following: aliquots of cells 2×10⁵ from total spleen cells, CD4+ and CD8+ fractions were stained with anti CD8-PE (53-6-7) and anti CD4-FITC (RM4.4) antibodies (PharMingen) for 15 minutes at room temperature. After washing, samples were analyzed on FACS CALIBUR (Bacton Dickinson). Spleen cells from naïve DRB1*0401-transgenic mouse were stained in a similar way as experimental samples and additionally with isotype control antibodies, single and double stained samples to adjust FACS CALUBUR settings.

ELISPOT Assay for IFN-Gamma Release from Murine Splenocytes

ELISpot assay plates (MAHA S4510, Millipore, Germany) were rinsed with PBS (200 μl-well), coated with anti-mouse IFN-gamma (INF-γ) mAb (clone R46A2 purchased from ATCC, Manassas, Va.; 50 μl-well of 1 μg/ml in 0.1 M NaHCO₃, pH 9.2-9.5) and incubated overnight at 4° C. Plates were washed four times with PBS containing 0.1% Tween-20 and incubated with PBS supplemented with 1% BSA (200 μl/well) at room temperature for two hours to block nonspecific binding. Cells were seeded at total amount of 1×10⁶ and 3.3×10⁵ and 1.1×10⁵ cells per well in 100 μl and incubated overnight at 37° C./5% CO₂ with 10 μg/ml (final concentration) of different stimulants added individually to the wells containing cells in volume of 100 μl: either long peptide used for vaccinations (relevant peptides) or overlapping 15-mers, derived from the longer peptide, or irrelevant peptide HA306-318 (no. 1171), not used for vaccination, or medium control. Subsequently, plates were washed four times and incubated with biotinylated anti-mouse IFN-γ mAb (clone AN18.17.24 purchased from ATCC, Manassas, Va.; 100 ml/well of 2 μg/ml in PBS/1% BSA) for two hours at 37° C. After washing, 100 μl/well of streptavidine-peroxidase diluted 1:5000 in PBS (Roche Diagnostics, Vienna, Austria) was added and plates were incubated at 37° C. for one additional hour. Afterwards, plates were washed four times with PBS/0.1% Tween-20 and 100 μl/well of the substrate (10 ml of 10 mM Tris pH 7.5 supplemented with 200 μl of 40 mg/ml DAB, 50 ml of 80 mg/ml NiCl₂ and 5 μl of 30% H₂O₂) was added. The reaction was stopped after 20-30 minutes by washing the plates with tap water. Dried plates were analyzed on BIOREADER 2000 (BioSys, Karben, Germany) and MICROSOFT OFFICE EXCEL program.

### Example I

### Epitope Capture from Peptide Mixtures and Identification by Mass Spectrometry

In this example the ability to capture high affinity peptides binding to HLA class II molecules from relatively simple peptide mixtures is demonstrated.

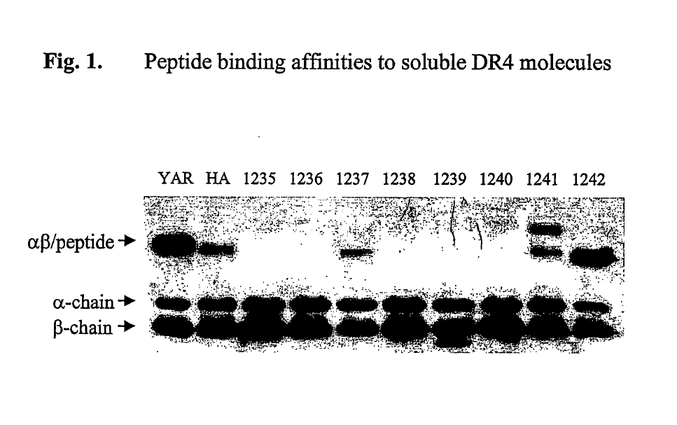

First, binding affinities of some individual peptides to soluble DRb1*0401 molecules were detected in a direct binding assay (FIG. 1). Peptide affinity was defined as high or low in comparison with binding of well-known “strong” binders, YAR and HA306-318 (Valli et al. (1993) in SDS-stability assay (Table 1). The binding affinity of 1242 peptide was considered to be the highest, comparable with affinity of YAR peptide.

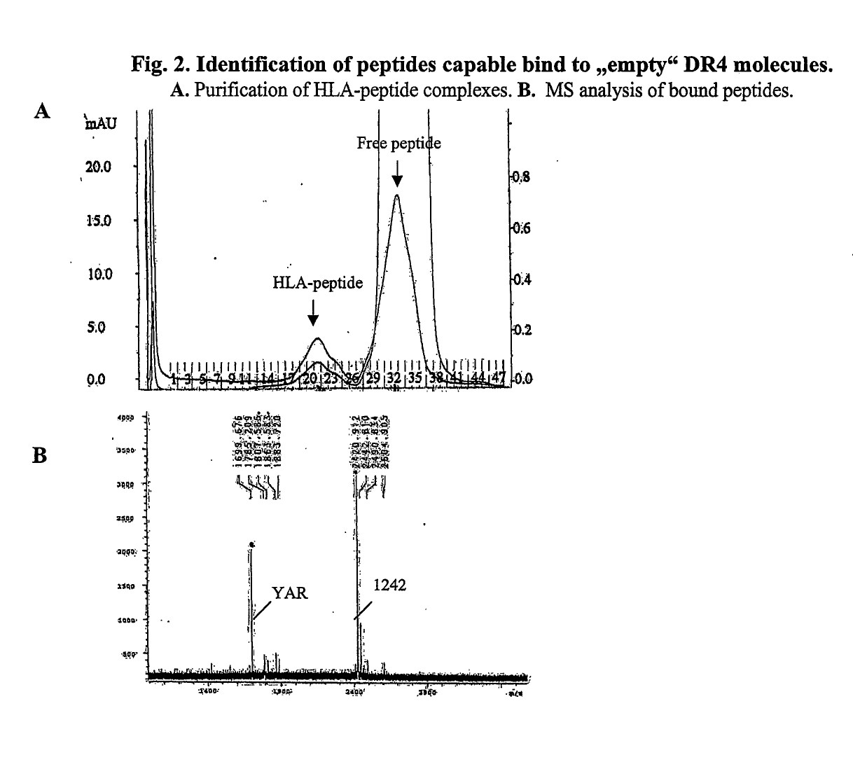

Second, the ability to capture peptides binding to HLA class II molecules from the mixture of two high affinity ligands was tested. The binding reaction contained 1 μM of soluble DRB1*0401 molecules and 5 μM of each YAR and 1242 peptide. After the HLA-peptide complexes were formed, they were separated from the excess of free peptides by gel filtration chromatography. Fractions containing MHC molecules were collected. Bound peptides were eluted from the complexes and analyzed by mass-spectrometry. As the read-out, both tested high affinity ligands were revealed in the complexes with MHC molecules (FIG. 2).

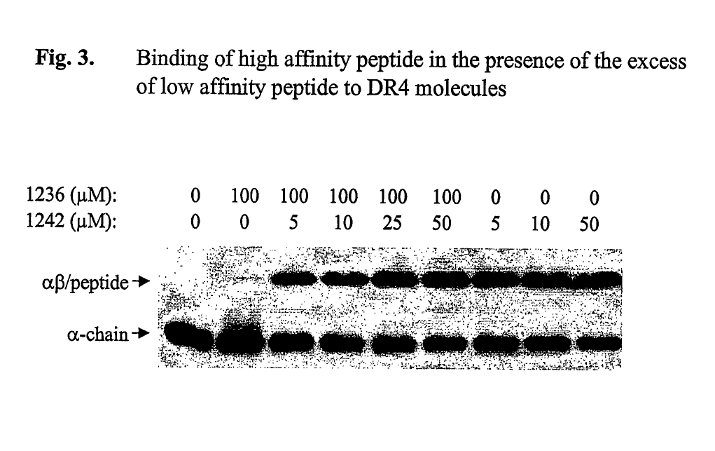

Third, it was investigated if the binding capacity of the high affinity ligand would be influenced by the excess of low affinity peptide. For this purpose, binding reaction of 1 μM DRB1*0401 molecules with 1242 peptide added in the concentration from 5 to 50 μM in the presence of constant amount (100 μM) of low affinity peptide 1236 was performed, so that the excess of 1236 over the 1242 peptide was 2-20 fold. Peptide binding was assessed by SDS-stability assay and verified by MS analysis (FIG. 3). This experiment shows that 20-fold molar excess of low affinity peptide does not impede the formation of stable MHC-complexes with high affinity ligand.

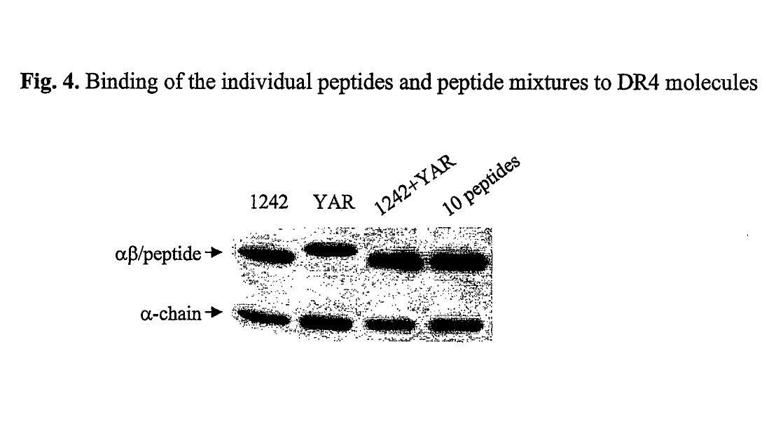

Finally, binding of the pool of 10 peptides of different binding affinities was tested (see Table 1). The final concentration of each peptide in binding reaction was 5 μM, except 1236 added in 55 μM, meaning that the concentration of each individual binding peptide was 20-fold lower than final concentration of all peptides. DRB1*0401 molecules were added to 1 μM. The formation of MHC-peptide complexes was evaluated as described above, using single peptides as controls (FIG. 4). Peptides captured by DR4 molecules were purified and sequenced by MS analysis. The result of this analysis yielded the two peptides with highest binding affinities: 1242 and YAR (see Table 1). The peptides with moderate and/or low affinities were not found in the complexes, probably due to the high concentration of strong competitors used in this assay. But when the concentration of binding peptides was reduced to 0.5 μM such that DR4 molecules were added in the excess over each peptide then the peptide of moderate affinity (HA306-318) was determined. These results demonstrated the possibility of capturing more then one high affinity peptides as well as peptides of moderate binding affinities from the peptide mixtures.

TABLE 1

| | | | |
|---|---|---|---|
| Peptide | | DR4 | |
| ID | Sequence | binding# | Reference |

| | | | |
|---|---|---|---|
| YAR | YARFQSQTTLKQKT (SEQ ID NO:16) | +++ | Valli et |
| | | | al., 1993 |
| HA₃₀₆₋₃₁₈ | YPKYVKQNTLKLAT (SEQ ID NO:17) | ++ | Valli et |
| | | al., 1993 | |
| 1235 | IDELKTNSSLLTSILTYHVV (SEQ ID NO:18) | − | |
| 1236 | TGSGAGIAQAAAGTV (SEQ ID NO:19) | + | |
| 1237 | GVSTANATVYMIDSVL (SEQ ID NO:20) | ++ | |
| 1238 | NFAGIEAAASAIQGNV (SEQ ID NO:21) | − | |
| 1239 | AETPGCVAYIGISFLDQ (SEQ ID NO:22) | − | |
| 1240 | VSDLKSSTAVIPGYPV (SEQ ID NO:23) | + | |
| 1241 | NFLLPDAQSIQAAAAG (SEQ ID NO:24) | ++ | |
| 1242 | YNINISLPSYYPDQKSL (SEQ ID NO:25) | +++ | |

#“+++”high binding affinity; “++”moderate binding affinity; “+”low binding affinity; “−”no binding.

### Example II

### Measuring T Cell Responses Against Captured Peptides by Synthetic T Cell Assays

In this example it is demonstrated, that peptides identified as described in Example I, not only bind to MHC molecules, but are also capable of stimulating T cells, proofing, that they are T cell epitopes. This question is relevant, because binding of a peptide to a MHC molecule by itself does not guarantee, that this peptide is relevant “in vivo”. The peptide might not be generated during “in vivo” antigen processing by the endosomal proteases (antigens for CD4+ T cells) or the proteasome (antigens for CD8+ T cells). And even peptides, which are processed and presented “in vivo” can instead of stimulating a T cell anergize it (antagonists). As a read out for T cell stimulation an IFN-γ ELISPOT assay was chosen.

For the example shown in FIG. 5a, the CD4+ T cells from healthy HCV-negative, BCG vaccinated donors were isolated from peripheral blood and cocultivated with sHLA DRB1*0401 loaded with p1242 and p84, an HCV peptide, which was used as a negative control. Costimulation was provided by adding an anti CD28 monoclonal antibody. FIG. 5a shows the number of induced IFN-γ spots, each spot representing a stimulated T cell. The induced number is significantly above the spontaneous IFN-γ secretion detected in the sample with the negative control peptide.

In order to show, that this not only holds true for MHC class II restricted peptides, the CD8+ T cells from healthy, HIV negative EBV-infected donors with a recent Influenza infection were incubated with sHLA A0201 molecules, loaded with either a peptide from the Influenza Matrix protein or the EBV BMLF1 antigen. Again a marked T cell response against both viral peptides could be detected (FIG. 5b). The specifity of this response was proven by incubating the loaded sHLA A0201 molecules with anti HLA A0201 antibody prior to cocultivation with the CD8+ T cells. This preincubation nearly totally abolished IFN-γ secretion, only spontaneous IFN-γ secretion was detected in these samples.

These examples demonstrate, that functional T cells against the captured peptides exist “in vivo”, that these peptides are therefore epitopes and might be useful in a vaccine, inducing protective immune responses.

### Example III

### Rapid Identification of HLA-Binding Peptides by Measuring Peptide Pools Arrayed in Matrix Format

After confirming the ability to use peptide pools for detection of individual binding peptides, the approach according to the present invention was applied to identify peptides capable to bind HLA class II molecules from CMV pp65 antigen. For this purpose, the direct peptide binding method was combined with the peptide pool array approach, described early (Kern et al., 1999; Tobery, et al., 2001).

In order to span the entire pp65 sequence of 561 aa long (SEQ ID NO:1), 547 15 mers, overlapping by 14 aa, were designed. Sequences of all peptides are shown in Table 2, wherein “Peptide no 1”—“Peptide no 547” are individually and respectively SEQ ID NO:26—SEQ ID NO:572.

TABLE 2

Sequences of overlapping 15-mer peptides
spanning the entire CMV pp65 sequence

| Peptide | |
|---|---|
| no | Sequence |

| | |
|---|---|
| 1 | MESRGRRCPEMISVL |
| 2 | ESRGRRCPEMISVLG |
| 3 | SRGRRCPEMISVLGP |
| 4 | RGRRCPEMISVLGPI |
| 5 | GRRCPEMISVLGPIS |
| 6 | RRCPEMISVLGPISG |
| 7 | RCPEMISVLGPISGH |
| 8 | CPEMISVLGPISGHV |
| 9 | PEMISVLGPISGHVL |
| 10 | EMISVLGPISGHVLK |
| 11 | MISVLGPISGHVLKA |
| 12 | ISVLGPISGHVLKAV |
| 13 | SVLGPISGHVLKAVF |
| 14 | VLGPISGHVLKAVFS |
| 15 | LGPISGHVLKAVFSR |
| 16 | GPISGHVLKAVFSRG |
| 17 | PISGHVLKAVFSRGD |
| 18 | ISGHVLKAVFSRGDT |
| 19 | SGHVLKAVFSRGDTP |
| 20 | GHVLKAVFSRGDTPV |
| 21 | HVLKAVFSRGDTPVL |
| 22 | VLKAVFSRGDTPVLP |
| 23 | LKAVFSRGDTPVLPH |
| 24 | KAVFSRGDTPVLPHE |
| 25 | AVFSRGDTPVLPHET |
| 26 | VFSRGDTPVLPHETR |
| 27 | FSRGDTPVLPHETRL |
| 28 | SRGDTPVLPHETRLL |
| 29 | RGDTPVLPHETRLLQ |
| 30 | GDTPVLPHETRLLQT |
| 31 | DTPVLPHETRLLQTG |
| 32 | TPVLPHETRLLQTGI |
| 33 | PVLPHETRLLQTGIH |
| 34 | VLPHETRLLQTGIHV |
| 35 | LPHETRLLQTGIHVR |
| 36 | PHETRLLQTGIHVRV |
| 37 | HETRLLQTGIHVRVS |
| 38 | ETRLLQTGIHVRVSQ |
| 39 | TRLLQTGIHVRVSQP |
| 40 | RLLQTGIHVRVSQPS |
| 41 | LLQTGIHVRVSQPSL |
| 42 | LQTGIHVRVSQPSLI |
| 43 | QTGIHVRVSQPSLIL |
| 44 | TGIHVRVSQPSLILV |
| 45 | GIHVRVSQPSLILVS |
| 46 | IHVRVSQPSLILVSQ |
| 47 | HVRVSQPSLILVSQY |
| 48 | VRVSQPSLILVSQYT |
| 49 | RVSQPSLILVSQYTP |
| 50 | VSQPSLILVSQYTPD |
| 51 | SQPSLILVSQYTPDS |
| 52 | QPSLILVSQYTPDST |
| 53 | PSLILVSQYTPDSTP |
| 54 | SLILVSQYTPDSTPC |
| 55 | LILVSQYTPDSTPCH |
| 56 | ILVSQYTPDSTPCHR |
| 57 | LVSQYTPDSTPCHRG |
| 58 | VSQYTPDSTPCHRGD |
| 59 | SQYTPDSTPCHRGDN |
| 60 | QYTPDSTPCHRGDNQ |
| 61 | YTPDSTPCHRGDNQL |
| 62 | TPDSTPCHRGDNQLQ |
| 63 | PDSTPCHRGDNQLQV |
| 64 | DSTPCHRGDNQLQVQ |
| 65 | STPCHRGDNQLQVQH |
| 66 | TPCHRGDNQLQVQHT |
| 67 | PCHRGDNQLQVQHTY |
| 68 | CHRGDNQLQVQHTYF |
| 69 | HRGDNQLQVQHTYFT |
| 70 | RGDNQLQVQHTYFTG |
| 71 | GDNQLQVQHTYFTGS |
| 72 | DNQLQVQHTYFTGSE |
| 73 | NQLQVQHTYFTGSEV |
| 74 | QLQVQHTYFTGSEVE |
| 75 | LQVQHTYFTGSEVEN |
| 76 | QVQHTYFTGSEVENV |
| 77 | VQHTYFTGSEVENVS |
| 78 | QHTYFTGSEVENVSV |
| 79 | HTYFTGSEVENVSVN |
| 80 | TYFTGSEVENVSVNV |
| 81 | YFTGSEVENVSVNVH |
| 82 | FTGSEVENVSVNVHN |
| 83 | TGSEVENVSVNVHNP |
| 84 | GSEVENVSVNVHNPT |
| 85 | SEVENVSVNVHNPTG |
| 86 | EVENVSVNVHNPTGR |
| 87 | VENVSVNVHNPTGRS |
| 88 | ENVSVNVHNPTGRSI |
| 89 | NVSVNVHNPTGRSIC |
| 90 | VSVNVHNPTGRSICP |
| 91 | SVNVHNPTGRSICPS |
| 92 | VNVHNPTGRSICPSQ |
| 93 | NVHNPTGRSICPSQE |
| 94 | VHNPTGRSICPSQEP |
| 95 | HNPTGRSICPSQEPM |
| 96 | NPTGRSICPSQEPMS |
| 97 | PTGRSICPSQEPMSI |
| 98 | TGRSICPSQEPMSIY |
| 99 | GRSICPSQEPMSIYV |
| 100 | RSICPSQEPMSIYVY |
| 101 | SICPSQEPMSIYVYA |
| 102 | ICPSQEPMSIYVYAL |
| 103 | CPSQEPMSIYVYALP |
| 104 | PSQEPMSIYVYALPL |
| 105 | SQEPMSIYVYALPLK |
| 106 | QEPMSIYVYALPLKM |
| 107 | EPMSIYVYALPLKML |
| 108 | PMSIYVYALPLKMLN |
| 109 | MSIYVYALPLKMLNI |
| 110 | SIYVYALPLKMLNIP |
| 111 | IYVYALPLKMLNIPS |
| 112 | YVYALPLKMLNIPSI |
| 113 | VYALPLKMLNIPSIN |
| 114 | YALPLKMLNIPSINV |
| 115 | ALPLKMLNIPSINVH |
| 116 | LPLKMLNIPSINVHH |
| 117 | PLKMLNIPSINVHHY |
| 118 | LKMLNIPSINVHHYP |
| 119 | KMLNIPSINVHHYPS |
| 120 | MLNIPSINVHHYPSA |
| 121 | LNIPSINVHHYPSAA |
| 122 | NIPSINVHHYPSAAE |
| 123 | IPSINVHHYPSAAER |
| 124 | PSINVHHYPSAAERK |
| 125 | SINVHHYPSAAERKH |
| 126 | INVHHYPSAAERKHR |
| 127 | NVHHYPSAAERKHRH |
| 128 | VHHYPSAAERKHRHL |
| 129 | HHYPSAAERKHRHLP |
| 130 | HYPSAAERKHRHLPV |
| 131 | YPSAAERKHRHLPVA |
| 132 | PSAAERKHRHLPVAD |
| 133 | SAAERKHRHLPVADA |
| 134 | AAERKHRHLPVADAV |
| 135 | AERKHRHLPVADAVI |
| 136 | ERKHRHLPVADAVIH |
| 137 | RKHRHLPVADAVIHA |
| 138 | KHRHLPVADAVIHAS |
| 139 | HRHLPVADAVIHASG |
| 140 | RHLPVADAVIHASGK |
| 141 | HLPVADAVIHASGKQ |
| 142 | LPVADAVIHASGKQM |
| 143 | PVADAVIHASGKQMW |
| 144 | VADAVIHASGKQMWQ |
| 145 | ADAVIHASGKQMWQA |
| 146 | DAVIHASGKQMWQAR |
| 147 | AVIHASGKQMWQARL |
| 148 | VIHASGKQMWQARLT |
| 149 | IHASGKQMWQARLTV |
| 150 | HASGKQMWQARLTVS |
| 151 | ASGKQMWQARLTVSG |
| 152 | SGKQMWQARLTVSGL |
| 153 | GKQMWQARLTVSGLA |
| 154 | KQMWQARLTVSGLAW |
| 155 | QMWQARLTVSGLAWT |
| 156 | MWQARLTVSGLAWTR |
| 157 | WQARLTVSGLAWTRQ |
| 158 | QARLTVSGLAWTRQQ |
| 159 | ARLTVSGLAWTRQQN |
| 160 | RLTVSGLAWTRQQNQ |
| 161 | LTVSGLAWTRQQNQW |
| 162 | TVSGLAWTRQQNQWK |
| 163 | VSGLAWTRQQNQWKE |
| 164 | SGLAWTRQQNQWKEP |
| 165 | GLAWTRQQNQWKEPD |
| 166 | LAWTRQQNQWKEPDV |
| 167 | AWTRQQNQWKEPDVY |
| 168 | WTRQQNQWKEPDVYY |
| 169 | TRQQNQWKEPDVYYT |
| 170 | RQQNQWKEPDVYYTS |
| 171 | QQNQWKEPDVYYTSA |
| 172 | QNQWKEPDVYYTSAF |
| 173 | NQWKEPDVYYTSAFV |
| 174 | QWKEPDVYYTSAFVF |
| 175 | WKEPDVYYTSAFVFP |
| 176 | KEPDVYYTSAFVFPT |
| 177 | EPDVYYTSAFVFPTK |
| 178 | PDVYYTSAFVFPTKD |
| 179 | DVYYTSAFVFPTKDV |
| 180 | VYYTSAFVFPTKDVA |
| 181 | YYTSAFVFPTKDVAL |
| 182 | YTSAFVFPTKDVALR |
| 183 | TSAFVFPTKDVALRH |
| 184 | SAFVFPTKDVALRHV |
| 185 | AFVFPTKDVALRHVV |
| 186 | FVFPTKDVALRHVVC |
| 187 | VFPTKDVALRHVVCA |
| 188 | FPTKDVALRHVVCAH |
| 189 | PTKDVALRHVVCAHE |
| 190 | TKDVALRHVVCAHEL |
| 191 | KDVALRHVVCAHELV |
| 192 | DVALRHVVCAHELVC |
| 193 | VALRHVVCAHELVCS |
| 194 | ALRHVVCAHELVCSM |
| 195 | LRHVVCAHELVCSME |
| 196 | RHVVCAHELVCSMEN |
| 197 | HVVCAHELVCSMENT |
| 198 | VVCAHELVCSMENTR |
| 199 | VCAHELVCSMENTRA |
| 200 | CAHELVCSMENTPAT |
| 201 | AHELVCSMENTRATK |
| 202 | HELVCSMENTRATKM |
| 203 | ELVCSMENTRATKMQ |
| 204 | LVCSMENTRATKMQV |
| 205 | VCSMENTRATKMQVI |
| 206 | CSMENTRATKMQVIG |
| 207 | SMENTRATKMQVIGD |
| 208 | MENTRATKMQVIGDQ |
| 209 | ENTRATKMQVIGDQY |
| 210 | NTRATKMQVIGDQYV |
| 211 | TRATKMQVIGDQYVK |
| 212 | RATKMQVIGDQYVKV |
| 213 | ATKMQVIGDQYVKVY |
| 214 | TKMQVIGDQYVKVYL |
| 215 | KMQVIGDQYVKVYLE |
| 216 | MQVIGDQYVKVYLES |
| 217 | QVIGDQYVKVYLESF |
| 218 | VIGDQYVKVYLESFC |
| 219 | IGDQYVKVYLESFCE |
| 220 | GDQYVKVYLESFCED |
| 221 | DQYVKVYLESFCEDV |
| 222 | QYVKVYLESFCEDVP |
| 223 | YVKVYLESFCEDVPS |
| 224 | VKVYLESFCEDVPSG |
| 225 | KVYLESFCEDVPSGK |
| 226 | VYLESFCEDVPSGKL |
| 227 | YLESFCEDVPSGKLF |
| 228 | LESFCEDVPSGKLFM |
| 229 | ESFCEDVPSGKLFMH |
| 230 | SFCEDVPSGKLFMHV |
| 231 | FCEDVPSGKLFMHVT |
| 232 | CEDVPSGKLFMHVTL |
| 233 | EDVPSGKLFMHVTLG |
| 234 | DVPSGKLFMHVTLGS |
| 235 | VPSGKLFMHVTLGSD |
| 236 | PSGKLFMHVTLGSDV |
| 237 | SGKLFMHVTLGSDVE |
| 238 | GKLFMHVTLGSDVEE |
| 239 | KLFMHVTLGSDVEED |
| 240 | LFMHVTLGSDVEEDL |
| 241 | FMHVTLGSDVEEDLT |
| 242 | MHVTLGSDVEEDLTM |
| 243 | HVTLGSDVEEDLTMT |
| 244 | VTLGSDVEEDLTMTR |
| 245 | TLGSDVEEDLTMTRN |
| 246 | LGSDVEEDLTMTRNP |
| 247 | GSDVEEDLTMTRNPQ |
| 248 | SDVEEDLTMTRNPQP |
| 249 | DVEEDLTMTRNPQPF |
| 250 | VEEDLTMTRNPQPFM |
| 251 | EEDLTMTRNPQPFMR |
| 252 | EDLTMTRNPQPFMRP |
| 253 | DLTMTRNPQPFMRPH |
| 254 | LTMTRNPQPFMRPHE |
| 255 | TMTRNPQPFMRPHER |
| 256 | MTRNPQPFMRPHERN |
| 257 | TRNPQPFMRPHERNG |
| 258 | RNPQPFMRPHERNGF |
| 259 | NPQPFMRPHERNGFT |
| 260 | PQPFMRPHERNGFTV |
| 261 | QPFMRPHERNGFTVL |
| 262 | PFMRPHERNGFTVLC |
| 263 | FMRPHERNGFTVLCP |
| 264 | MRPHERNGFTVLCPK |
| 265 | RPHERNGFTVLCPKN |
| 266 | PHERNGFTVLCPKNM |
| 267 | HERNGFTVLCPKNNI |
| 268 | ERNGFTVLCPKNMII |
| 269 | RNGFTVLCPKNMIIK |
| 270 | NGFTVLCPKJSIMII |
| 271 | GFTVLCPKNMIIKPG |
| 272 | FTVLCPKNMIIKPGK |
| 273 | TVLCPKNMIIKPGKI |
| 274 | VLCPKNNIIKPGKIS |
| 275 | LCPKNMIIKPGKISH |
| 276 | CPKNMIIKPGKISHI |
| 277 | PKNNIIKPGKISHIM |
| 278 | KNNIIKPGKISHIML |
| 279 | NMIIKPGKISHIMLD |
| 280 | MIIKPGKISHIMLDV |
| 281 | IIKPGKISHIMLDVA |
| 282 | IKPGKISHIMLDVAF |
| 283 | KPGKISHIMLDVAFT |
| 284 | PGKISHIMLDVAFTS |
| 285 | GKISHIMLDVAFTSH |
| 286 | KISHIMLDVAFTSHE |
| 287 | ISHIMLDVAFTSHEH |
| 288 | SHIMLDVAFTSHEHF |
| 289 | HIMLDVAFTSHEHFG |
| 290 | IMLDVAFTSHEHFGL |
| 291 | MLDVAFTSHEHFGLL |
| 292 | LDVAFTSHEHFGLLC |
| 293 | DVAFTSHEHFGLLCP |
| 294 | VAFTSHEHFGLLCPK |
| 295 | AFTSHEHFGLLCPKS |
| 296 | FTSHEHFGLLCPKSI |
| 297 | TSHEHFGLLCPKSIP |
| 298 | SHEHFGLLCPKSIPG |
| 299 | HEHFGLLCPKSIPGL |
| 300 | EHFGLLCPKSIPGLS |
| 301 | HFGLLCPKSIPGLSI |
| 302 | FGLLCPKSIPGLSIS |
| 303 | GLLCPKSIPGLSISG |
| 304 | LLCPKSIPGLSISGN |
| 305 | LCPKSIPGLSISGNL |
| 306 | CPKSIPGLSISGNLL |
| 307 | PKSIPGLSISGNLLM |
| 308 | KSIPGLSISGNLLMN |
| 309 | SIPGLSISGNLLMMG |
| 310 | IPGLSISGNLLMNGQ |
| 311 | PGLSISGNLLMNGQQ |
| 312 | GLSISGNLLMNGQQI |
| 313 | LSISGNLLMNGQQIF |
| 314 | SISGNLLMNGQQIFL |
| 315 | ISGNLLMNGQQIFLE |
| 316 | SGNLLMNGQQIFLEV |
| 317 | GNLLMNGQQIFLEVQ |
| 318 | NLLMNGQQIFLEVQA |
| 319 | LLMNGQQIFLEVQAI |
| 320 | LMNGQQIFLEVQAIR |
| 321 | MNGQQIFLEVQAIRE |
| 322 | NGQQIFLEVQAIRET |
| 323 | GQQIFLEVQAIRETV |
| 324 | QQIFLEVQAIRETVE |
| 325 | QIFLEVQAIRETVEL |
| 326 | IFLEVQAIRETVELR |
| 327 | FLEVQAIRETVELRQ |
| 328 | LEVQAIRETVELRQY |
| 329 | EVQAIRETVELRQYD |
| 330 | VQAIRETVELRQYDP |
| 331 | QAIRETVELRQYDPV |
| 332 | AIRETVELRQYDPVA |
| 333 | IRETVELRQYDPVAA |
| 334 | RETVELRQYDPVAAL |
| 335 | ETVELRQYDPVAAJJ |
| 336 | TVELRQYDPVAALFF |
| 337 | VELRQYDPVAALFFF |
| 338 | ELRQYDPVAALFFFD |
| 339 | LRQYDPVAALFFFDI |
| 340 | RQYDPVAALFFFDID |
| 341 | QYDPVAALFFFDIDL |
| 342 | YDPVAALFFFDIDLL |
| 343 | DPVAALFFFDIDLLL |
| 344 | PVAALFFFDIDLLLQ |
| 345 | VAALFFFDIDLLLQR |
| 346 | AALFFFDIDLLLQRG |
| 347 | ALFFFDIDLLLQRGP |
| 348 | LFFFDIDLLLQRGPQ |
| 349 | FFFDIDLLLQRGPQY |
| 350 | FFDIDLLLQRGPQYS |
| 351 | FDIDLLLQRGPQYSE |
| 352 | DIDLLLQRGPQYSEH |
| 353 | IDLLLQRGPQYSEHP |
| 354 | DLLLQRGPQYSEHPT |
| 355 | LLLQRGPQYSEHPTF |
| 356 | LLQRGPQYSEHPTFT |
| 357 | LQRGPQYSEHPTFTS |
| 358 | QRGPQYSEHPTFTSQ |
| 359 | RGPQYSEHPTFTSQY |
| 360 | GPQYSEHPTFTSQYR |
| 361 | PQYSEHPTFTSQYRI |
| 362 | QYSEHPTFTSQYRIQ |
| 363 | YSEHPTFTSQYRIQG |
| 364 | SEHPTFTSQYRIQGK |
| 365 | EHPTFTSQYRIQGKL |
| 366 | HPTFTSQYRIQGKLE |
| 367 | PTFTSQYRIQGKLEY |
| 368 | TFTSQYRIQGKLEYR |
| 369 | FTSQYRIQGKLEYRH |
| 370 | TSQYRIQGKLEYRHT |
| 371 | SQYRIQGKLEYRHTW |
| 372 | QYRIQGKLEYRHTWD |
| 373 | YRIQGKLEYRHTWDR |
| 374 | RIQGKLEYRHTWDRH |
| 375 | IQGKLEYRHTWDRHD |
| 376 | QGKLEYRHTWDRHDE |
| 377 | GKLEYRHTWDRHDEG |
| 378 | KLEYRHTWDRHDEGA |
| 379 | LEYRHTWDRHDEGAA |
| 380 | EYRHTWDRHDEGAAQ |
| 381 | YRHTWDRHDEGAAQG |
| 382 | RHTWDRHDEGAAQGD |
| 383 | HTWDRHDEGAAQGDD |
| 384 | TWDRHDEGAAQGDDD |
| 385 | WDRHDEGAAQGDDDV |
| 386 | DRHDEGAAQGDDDVW |
| 387 | RHDEGAAQGDDDVWT |
| 388 | HDEGAAQGDDDVWTS |
| 389 | DEGAAQGDDDVWTSG |
| 390 | EGAAQGDDDVWTSGS |
| 391 | GAAQGDDDVWTSGSD |
| 392 | AAQGDDDVWTSGSDS |
| 393 | AQGDDDVWTSGSDSD |
| 394 | QGDDDVWTSGSDSDE |
| 395 | GDDDVWTSGSDSDEE |
| 396 | DDDVWTSGSDSDEEL |
| 397 | DDVWTSGSDSDEELV |
| 398 | DVWTSGSDSDEELVT |
| 399 | VWTSGSDSDEELVTT |
| 400 | WTSGSDSDEELVTTE |
| 401 | TSGSDSDEELVTTER |
| 402 | SGSDSDEELVTTERK |
| 403 | GSDSDEELVTTERKT |
| 404 | SDSDEELVTTERKTP |
| 405 | DSDEELVTTERKTPR |
| 406 | SDEELVTTERKTPRV |
| 407 | DEELVTTERKTPRVT |
| 408 | EELVTTERKTPRVTG |
| 409 | ELVTTERKTPRVTGG |
| 410 | LVTTERKTPRVTGGG |
| 411 | VTTERKTPRVTGGGA |
| 412 | TTERKTPRVTGGGAM |
| 413 | TERKTPRVTGGGANA |
| 414 | ERKTPRVTGGGAMAG |
| 415 | RKTPRVTGGGAMAGA |
| 416 | KTPRVTGGGANAGAS |
| 417 | TPRVTGGGAMAGAST |
| 418 | PRVTGGGAMAGASTS |
| 419 | RVTGGGAMAGASTSA |
| 420 | VTGGGAMAGASTSAG |
| 421 | TGGGAMAGASTSAGR |
| 422 | GGGAMAGASTSAGRK |
| 423 | GGAIAGASTSAGRKR |
| 424 | GAMAGASTSAGRKRK |
| 425 | AMAGASTSAGRKRKS |
| 426 | MAGASTSAGRKRKSA |
| 427 | AGASTSAGRKRKSAS |
| 428 | GASTSAGRKRKSASS |
| 429 | ASTSAGRKRKSASSA |
| 430 | STSAGRKRKSASSAT |
| 431 | TSAGRKRKSASSATA |
| 432 | SAGRKRKSASSATAC |
| 433 | AGRKRKSASSATACT |
| 434 | GRKRKSASSATACTS |
| 435 | RKRKSASSATACTSG |
| 436 | KRKSASSATACTSGV |
| 437 | RKSASSATACTSGVM |
| 438 | KSASSATACTSGVMT |
| 439 | SASSATACTSGVMTR |
| 440 | ASSATACTSGVMTRG |
| 441 | SSATACTSGVMTRGR |
| 442 | SATACTSGVMTRGRL |
| 443 | ATACTSGVMTRGRLK |
| 444 | TACTSGVMTRGRLKA |
| 445 | ACTSGVMTRGRLKAE |
| 446 | CTSGVMTRGRLKAES |
| 447 | TSGVMTRGRLKAEST |
| 448 | SGVMTRGRLKAESTV |
| 449 | GVMTRGRLKAESTVA |
| 450 | VMTRGRLKAESTVAP |
| 451 | MTRGRLKAESTVAPE |
| 452 | TRGRLKAESTVAPEE |
| 453 | RGRLKAESTVAPEED |
| 454 | GRLKAESTVAPEEDT |
| 455 | RLKAESTVAPEEDTD |
| 456 | LKAESTVAPEEDTDE |
| 457 | KAESTVAPEEDTDED |
| 458 | AESTVAPEEDTDEDS |
| 459 | ESTVAPEEDTDEDSD |
| 460 | STVAPEEDTDEDSDN |
| 461 | TVAPEEDTDEDSDNE |
| 462 | VAPEEDTDEDSDNEI |
| 463 | APEEDTDEDSDNEIH |
| 464 | PEEDTDEDSDNEIHN |
| 465 | EEDTDEDSDNEIHNP |
| 466 | EDTDEDSDNEIHNPA |
| 467 | DTDEDSDNEIHNPAV |
| 468 | TDEDSDNEIHNPAVF |
| 469 | DEDSDNEIHNPAVFT |
| 470 | EDSDNEIHNPAVFTW |
| 471 | DSDNEIHNPAVFTWP |
| 472 | SDNEIHNPAVFTWPP |
| 473 | DNEIHNPAVFTWPPW |
| 474 | NEIHNPAVFTWPPWQ |
| 475 | EIHNPAVFTWPPWQA |
| 476 | IHNPAVFTWPPWQAG |
| 477 | HNPAVFTWPPWQAGI |
| 478 | NPAVFTWPPWQAGIL |
| 479 | PAVFTWPPWQAGILA |
| 480 | AVFTWPPWQAGILAR |
| 481 | VFTWPPWQAGILARN |
| 482 | FTWPPWQAGILARNL |
| 483 | TWPPWQAGILARNLV |
| 484 | WPPWQAGILARNLVP |
| 485 | PPWQAGILARNLVPM |
| 486 | PWQAGILARNLVPMV |
| 487 | WQAGILARNLVPMVA |
| 488 | QAGILARNLVPMVAT |
| 489 | AGILARNLVPMVATV |
| 490 | GILARNLVPMVATVQ |
| 491 | ILARNLVPMVATVQG |
| 492 | LARNLVPMVATVQGQ |
| 493 | ARNLVPMVATVQGQN |
| 494 | RNLVPMVATVQGQNL |
| 495 | NLVPMVATVQGQNLK |
| 496 | LVPMVATVQGQNLKY |
| 497 | VPMVATVQGQNLKYQ |
| 498 | PMVATVQGQNLKYQE |
| 499 | MVATVQGQNLKYQEF |
| 500 | VATVQGQNLKYQEFF |
| 501 | ATVQGQNLKYQEFFW |
| 502 | TVQGQNLKYQEFFWD |
| 503 | VQGQNLKYQEFFWDA |
| 504 | QGQNLKYQEFFWDAN |
| 505 | GQNLKYQEFFWDAND |
| 506 | QNLKYQEFFWDANDI |
| 507 | NLKYQEFFWDANDIY |
| 508 | LKYQEFFWDANDIYR |
| 509 | KYQEFFWDANDIYRI |
| 510 | YQEFFWDANDIYRIF |
| 511 | QEFFWDANDIYRIFA |
| 512 | EFFWDANDIYRIFAE |
| 513 | FFWDANDIYRIFAEL |
| 514 | FWDANDIYRIFAELE |
| 515 | WDANDIYRIFAELEG |
| 516 | DANDIYRIFAELEGV |
| 517 | ANDIYRIFAELEGVW |
| 518 | NDIYRIFAELEGVWQ |
| 519 | DIYRIFAELEGVWQP |
| 520 | IYRIFAELEGVWQPA |
| 521 | YRIFAELEGVWQPAA |
| 522 | RIFAELEGVWQPAAQ |
| 523 | IFAELEGVWQPAAQP |
| 524 | FAELEGVWQPAAQPK |
| 525 | AELEGVWQPAAQPKR |
| 526 | ELEGVWQPAAQPKRR |
| 527 | LEGVWQPAAQPKRRR |
| 528 | EGVWQPAAQPKRRRH |
| 529 | GVWQPAAQPKRRRHR |
| 530 | VWQPAAQPKRRRHRQ |
| 531 | WQPAAQPKRRRHRQD |
| 532 | QPAAQPKRRRHRQDA |
| 533 | PAAQPKRRRHRQDAL |
| 534 | AAQPKRRRHRQDALP |
| 535 | AQPKRRRHRQDALPG |
| 536 | QPKRRRHRQDALPGP |
| 537 | PKRRRHRQDALPGPC |
| 538 | KRRRHRQDALPGPCI |
| 539 | RRRHRQDALPGPCIA |
| 540 | RRHRQDALPGPCIAS |
| 541 | RHRQDALPGPCIAST |
| 542 | HRQDALPGPCIASTP |
| 543 | RQDALPGPCIASTPK |
| 544 | QDALPGPCIASTPKK |
| 545 | DALPGPCIASTPKKH |
| 546 | ALPGPCIASTPKKHR |
| 547 | LPGPCIASTPKKHRG |

Because of the huge peptide number to be tested, peptides were combined in 42 pools each containing 21 peptides arrayed in a matrix format, as shown on FIG. 6. The mixtures were prepared such that each individual peptide was contained in exactly one “row” pool and one “column” pool.

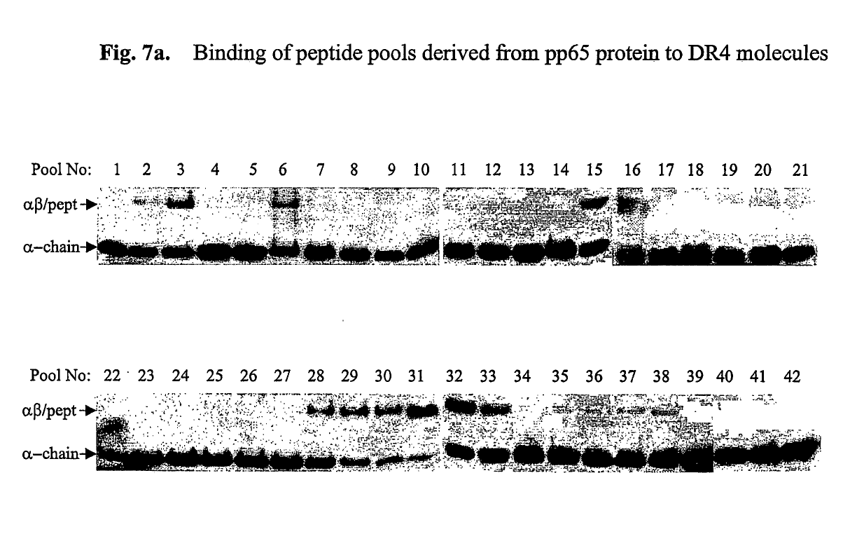

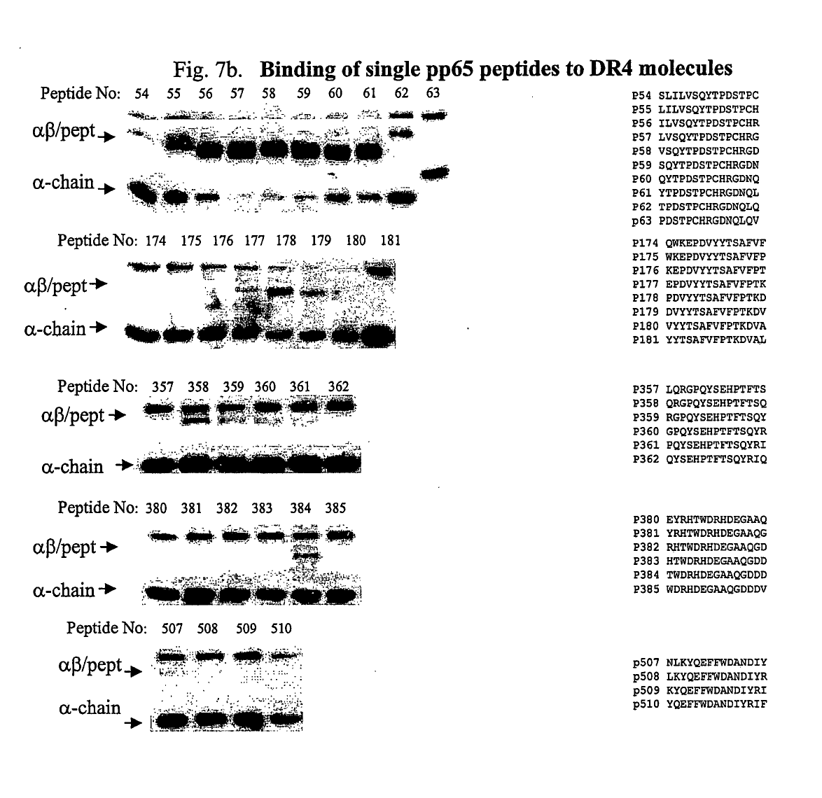

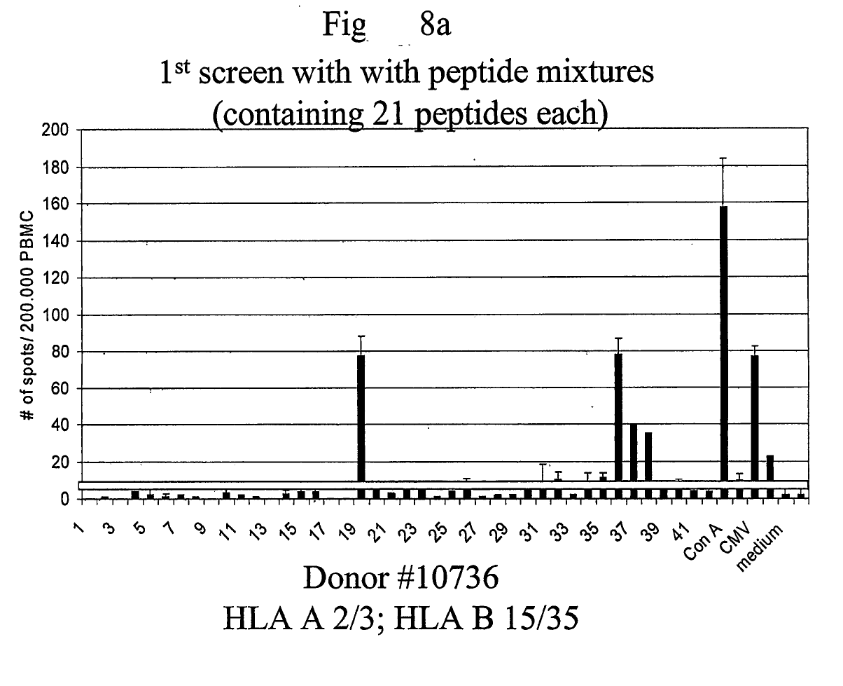

Each matrix pool was tested for binding affinity to DR4 molecules in SDS-stability assay; HA306-318 was used as a reference peptide (FIG. 7a). In the first screen 18 out of 42 positive peptide pools were found: no. 2, 3, 6, 15, 16, 19, 20 from “row” pools and no. 28-38 from “column” pools (see FIG. 6). By finding the intersections of reactive pools in the array, the 77 peptides were determined as potential binders. Then each single 15 mer was checked for binding to DR4 molecules individually (FIG. 7b, Table 3). As a result, 20 peptides selected in the first screen were confirmed to be binders in the second screen. Usually, in rows of the array more than one positive peptides was found. Probably, these peptides overlapping by 14 aa represent longer epitopes recognized by TCR receptors on CD4+ T cells.

Four peptides (peptide no. 177, 361, 507, 508) containing partially or fully identical sequences were described earlier as capable to evoke specific CD4+ T cells response in healthy, CMV-exposed human subjects (Khattab et al., 1997; Bitmansour et al., 2001). This confirms the validity of our approach.

Some of the identified DRB1*0401 binding peptides, as well as others chosen by prediction algorithms were also tested for binding to soluble HLA molecules of different allels: DRB1*0101, DRB1*0404, DRB1*0701 and DRB1*1101 (Table 3). Seven peptides (No 58, 177, 559, 360, 452, 470 and No 496) were demonstrated to have affinity for at least two HLA molecules arguing for promiscuity of these epitopes.

Three peptides binding to DRB1*0404 (no. 421, 469 and 470) were also found to induce IFN-gamma secretion in T cells from CMV seropositive donors (see Example IV) again indicating that also this second reverse immunological approach can identify true T cell epitopes.

In conclusion, several novel sequences derived from the CMV pp65 antigen, competent of binding to HLA class II molecules, considered of being candidates for class II epitopes (shaded in Table 3) were identified. Immunogenicity of peptides, incorporating mentioned epitopes, as well as their CD4+ specificity was verified by testing their ability to induce IFN-gamma production upon ex vivo stimulation of T cell from CMV seropositive donors and/or HLA-DRB1*0401 transgenic mice (see Example IV, VI and VII).

TABLE 3
Binding of individual 15mer peptides of CMV pp65 protein to
soluble HLA-molecules. Newly identified binders are shaded.

| | | | Binding # | Binding | Binding | |
|---|---|---|---|---|---|---|
| Mixture | Peptide | Peptide | to DRB1 | to DRB1 | to DRBl | Reference, |
| No. | No | sequence | *0401 | *0404 | *0101 | comment |

| | | | | | | |
|---|---|---|---|---|---|---|
| 2 and 31 | 37 | HETRLLQTGIHVRVS | − | nd | nd | Gallot et al., |
| | | (SEQ ID NO:62) | | | | CD4+clone / |
| | | | | | | DQ*06 02 |
| 2 and 35 | 41 | LLQTGIHVRVSQPSL | − | nd | nd | Gallot et al., |
| | | (SEQ ID NO:66) | | | | CD4+clone / |
| | | | | | | DQ*0602 |
| 2 and 37 | 43 | QTGIHVRVSQPSLIL | − | nd | nd | Gallot et al., |
| | | (SEQ ID NO:68) | | | | CD4+clone / |
| | | | | | | DQ*0602 |
| 3 and 27 | 54 | SLILVSQYTPDSTPC | − | nd | nd | |
| | | (SEQ ID NO:79) | | | | |
| 3 and 28 | 55 | LILVSQYTPDSTPCH | + | nd | nd | new, claim |
| | | (SEQ ID NO:80) | | | | |
| 3 and 29 | 56 | ILVSQYTPDSTPCHR | ++ | nd | nd | new, claim |
| | | (SEQ ID NO:81) | | | | |
| 3 and 30 | 57 | LVSQYTPDSTPCHRG | ++ | − | − | new, claim |
| | | (SEQ ID NO:82) | | | | |
| 3 and 31 | 58 | VSQYTPDSTPCHRGD | ++ | nd | nd | new, claim |
| | | (SEQ ID NO:83) | | | | |
| 3 and 32 | 59 | SQYTPDSTPCHRGDN | ++ | nd | nd | new, claim |
| | | (SEQ ID NO:84) | | | | |
| 3 and 33 | 60 | QYTPDSTPCHRGDNQ | ++ | nd | nd | new, claim |
| | | (SEQ ID NO:85) | | | | |
| 3 and 34 | 61 | YTPDSTPCHRGDNQL | ++ | nd | nd | new, claim |
| | | (SEQ ID NO:86) | | | | |
| 3 and 35 | 62^{§} | TPDSTPCHRGDNQLQ | − | nd | nd | new, claim |
| | | (SEQ ID NO:87) | | | | |
| 3 and 36 | 63^{§} | PDSTPCHRGDNQLQV | − | nd | nd | new, claim |
| | | (SEQ ID NO:88) | | | | |
| 3 and 37 | 64 | DSTPCHRGDNQLQVQ | + | nd | nd | new, claim |
| | | (SEQ ID NO:89) | | | | |
| 3 and 38 | 65 | STPCHRGDNQLQVQH | − | nd | nd | |
| | | (SEQ ID NO:90) | | | | |
| 4 and 23 | 107 | EPMSIYVYALPLKML | − | nd | − | |
| | | (SEQ ID NO:132) | | | | |
| 4 and 25 | 109 | SIYVYALPLKMLNIP | − | nd | + | |
| | | (SEQ ID NO:134) | | | | |
| 4 and 27 | 111 | IYVYALPLKMLNIPS | − | nd | − | |
| | | (SEQ ID NO:136) | | | | |
| 6 and 29 | 170 | RQQNQWKEPDVYYTS | − | nd | nd | |
| | | (SEQ ID NO:195) | | | | |
| 6 and 31 | 172 | QNQWKEPDVYYTSAF | − | nd | nd | |
| | | (SEQ ID NO:197) | | | | |
| 6 and 33 | 174 | QWKEPDVYYTSAFVF | − | nd | − | |
| | | (SEQ ID NO:199) | | | | |
| 6 and 34 | 175 | WKEPDVYYTSAFVFP | − | nd | + | |
| | | (SEQ ID NO:200) | | | | |
| 6 and 35 | 176 | KEPDVYYTSAFVFPT | +/− | nd | − | |
| | | (SEQ ID NO:201) | | | | |
| 6 and 36 | 177 | EPDVYYTSAFVFPTK | + | nd | nd | Bitmansour et |
| | | (SEQ ID NO:202) | al., CD4+ clone | | | |
| 6 and 37 | 178 | PDVYYTSAFVFPTKD | ++ | nd | nd | |
| | | (SEQ ID NO:203) | | | | |
| 6 and 38 | 179 | DVYYTSAFVFPTKDV | + | nd | nd | |
| | | (SEQ ID NO:204) | | | | |
| 6 and 39 | 180 | VYYTSAFVFPTKDVA | +/− | nd | nd | |
| | | (SEQ ID NO:205) | | | | |
| 15 and 27 | 357 | LQRGPQYSEHPTFTS | − | nd | nd | |
| | | (SEQ ID NO:382) | | | | |
| 15 and 28 | 358 | QRGPQYSEHPTFTSQ | + | nd | nd | |
| | | (SEQ ID NO:383) | | | | |
| 15 and 29 | 359 | RGPQYSEHPTFTSQY | +/− | nd | nd | |
| | | (SEQ ID NO:384) | | | | |
| 15 and 30 | 360 | GPQYSEHPTFTSQYR | +/− | nd | nd | |
| | | (SEQ ID NO:385) | | | | |
| 15 and 31 | 361 | PQYSEHPTFTSQYRI | +/− | nd | nd | Khattab et al., |
| | | (SEQ ID NO:386) | | | | CD4+ clone / |
| | | | | | | DR11 |
| 15 and 32 | 362 | QYSEHPTFTSQYRIQ | − | nd | nd | |
| | | (SEQ ID NO:387) | | | | |
| 15 and 33 | 363 | YSEHPTFTSQYRIQG | − | nd | nd | |
| | | (SEQ ID NO:388) | | | | |
| 15 and 35 | 365 | EHPTFTSQYRIQGKL | − | nd | nd | Gallot et al., |
| | | (SEQ ID NO:390) | | | | CD4+ clone / |
| | | | | | | DR*1302 |
| 15 and 37 | 367 | PTFTSQYRIQGKLEY | − | nd | nd | Gallot et al., |
| | | (SEQ ID NO:392) | | | | CD4+ clone / |
| | | | | | | DR*1302 |
| 16 and 28 | 379 | LEYRHTWDRHDEGAA | − | nd | nd | |
| | | (SEQ ID NO:404) | | | | |
| 16 and 29 | 380 | EYRHTWDRHDEGAAQ | − | nd | nd | |
| | | (SEQ ID NO:405) | | | | |
| 16 and 31 | 382 | RHTWDRHDEGAAQGD | − | nd | nd | |
| | | (SEQ ID NO:407) | | | | |
| 16 and 32 | 383 | HTWDRHDEGAAQGDD | +/− | nd | nd | new, claim |
| | | (SEQ ID NO:408) | | | | |
| 16 and 33 | 384 | TWDRHDEGAAQGDDD | + | nd | nd | new, claim |
| | | (SEQ ID NO:409) | | | | |
| 16 and 34 | 385 | WDRHDEGAAQGDDDV | − | nd | nd | |
| | | (SEQ ID NO:410) | | | | |
| 16 and 37 | 388 | HDEGAAQGDDDVWTS | − | nd | nd | |
| | | (SEQ ID NO:413) | | | | |
| 18 and 26 | 419 | RVTGGGAMAGASTSA | − | − | nd | |
| | | (SEQ ID NO:444) | | | | |
| 18 and 28 | 421 | TGGGAMAGASTSAGR | − | + | nd | |
| | | (SEQ ID NO:446) | | | | |
| 18 and 30 | 423 | GGAMAGASTSAGRKR | − | − | nd | |
| | | (SEQ ID NO:448) | | | | |
| nd | 448 | SGVMTRGRLKAESTV | − | nd | nd | |
| | | (SEQ ID NO:473) | | | | |
| nd | 449 | GVMTRGRLKAESTVA | +/− | nd | nd | |
| | | (SEQ ID NO:474) | | | | |
| nd | 450 | VMTRGRLKAESTVAP | + | nd | nd | |
| | | (SEQ ID NO:475) | | | | |
| nd | 451 | MTRGRLKAESTVAPE | + | nd | nd | |
| | | (SEQ ID NO:476) | | | | |
| nd | 452 | TRGRLKAESTVAPEE | + | + | nd | |
| | | (SEQ ID NO:477) | | | | |
| nd | 453 | RGRLKAESTVAPEED | + | nd | nd | |
| | | (SEQ ID NO:478) | | | | |
| nd | 454 | GRLKAESTVAPEEDT | + | nd | nd | |
| | | (SEQ ID NO:479) | | | | |
| nd | 455 | RLKAESTVAPEEDTD | − | nd | nd | |
| | | (SEQ ID NO:480) | | | | |
| 18 and 35 | 468 | TDEDSDNEIHNPAVF | − | − | nd | |
| | | (SEQ ID NO:493) | | | | |
| 18 and 36 | 469 | DEDSDNEIHNPAVFT | − | +/− | nd | |
| | | (SEQ ID NO:494) | | | | |
| 18 and 37 | 470 | EDSDNEIHNPAVFTW | − | +/− | nd | |
| | | (SEQ ID NO:495) | | | | |
| 19 and 38 | 492 | LARNLVPMVATVQGQ | − | ++ | | |
| | | (SEQ ID NO:517) | | | | |
| 19 and 40 | 494 | RNLVPMVATVQGQNL | − | ++ | | |
| | | (SEQ ID NO:519) | | | | |
| 19 and 42 | 496 | LVPMVATVQGQNLKY | − | + | + | |
| | | (SEQ ID NO:521) | | | | |
| 20 and 32 | 507 | NLKYQEFFWDANDIY | +/− | nd | nd | Bitmansour et |
| | | (SEQ ID NO:532) | | | | el., CD4+ clone |
| 20 and 33 | 508 | LKYQEFFWDANDIYR | +/− | nd | nd | Bitmansour |
| | | (SEQ ID NO:533) | | | | Khattab, CD4+ / |
| | | | | | | DR3 |
| 20 and 35 | 510 | YQEFFWDANDIYRIF | − | nd | nd | Khattab et al., |
| | | (SEQ ID NO:535) | | | | CD4+ / DR3 |
| 20 and 37 | 512 | EFFWDANDIYRIFAE | − | nd | nd | |
| | | (SEQ ID NO:537) | | | | |

# “−” no binding; “+/−” weak binding; “+“ intermediate binding; “++” strong binding; “nd” not determined. § peptide is not soluble in water.

### Example IV

### Identification of T Cell Epitopes by Measuring Cytokine Responses to 15-mer Peptide Pools Arrayed as Matrix and Confirmation of Individual Peptides

547 peptides, each individual peptide consisting of 15 aa and overlapping its precursor by 14 out of 15 aa, were designed to span the complete sequence of the CMV pp65 antigen. Sequences of all peptides are shown in Table 2.

Because the number of available human PBMC for screening T cell responses is usually limited, peptides were not applied individually, but combined to either column-pools or row-pools arrayed in a matrix format (FIG. 6). First, individual peptides were dissolved in 100% DMSO at 5 mg/ml. Then mixtures of 21 peptides each were prepared as such that each individual peptide was contained in exactly 2 pools. For restimulation of human PBMCs peptide mixtures were diluted in assay medium such that each peptide was present at a final concentration of 5 μg/ml. This concentration is well above the concentration of 1.75 μg/ml, which Maecker et al. (Maecker paper) found to be saturating for CD4 and CD8 positive T cell responses. The final concentration of DMSO in each sample was 2.1%.

Altogether PBMC from 10 healthy, CMV seropositive, HLA-A2 positive donors were used for screening of the 42 peptide pools represented in FIG. 6. To verify, whether class I epitopes usually shorter than 15 aa were properly recognized within the 15mer peptides and that the high DMSO content did not impair T cell activation and function, a single well-characterized immunodominant CD8-restricted 9-mer epitope from CMV pp65 (NLVPMVATV (SEQ ID NO:573), pos. 495-503) was included in each screen at a concentration of 10 μg/ml (final DMSO concentration 0.1%).

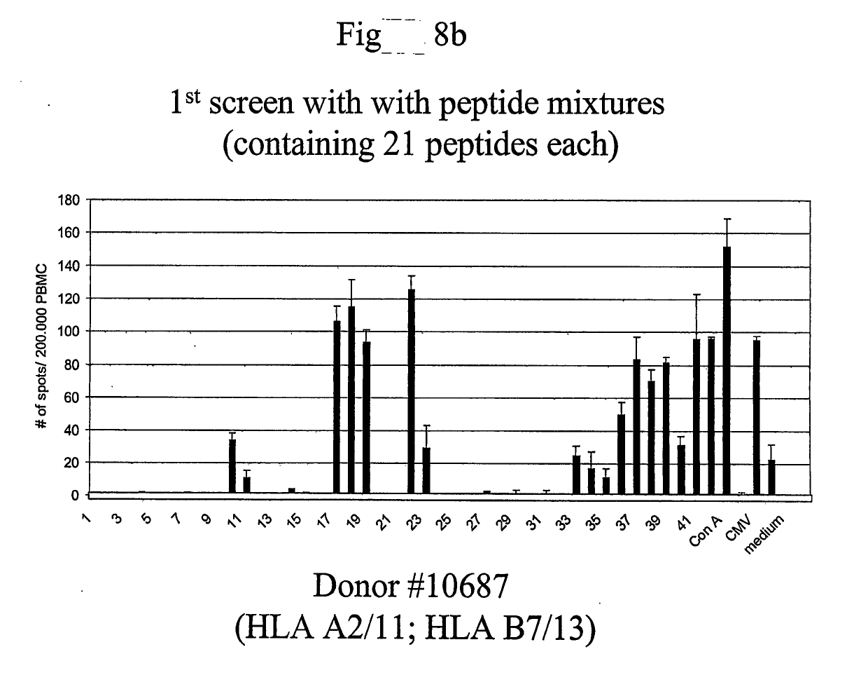

As a read out for T cell reactivation a human IFN-gamma Elispot was used. FIG. 8a shows a donor reacting with very few peptide pools. The crossover points of the positive column and row pools identify peptides 490-492. These 3 peptides contain the well-characterized immunodominant CD8-restricted 9-mer minimal epitope NLVPMVATV (SEQ ID NO:573), pos. 495-503. Responses elicited by the peptide pools were largely equal to the response elicited with 9-mer minimal epitope (FIG. 8a). This indicates, that shorter epitopes are properly recognized within 15-mer peptides and that the high DMSO content did not impair the T cell response.

Most other donors showed a more complex response directed against several epitopes. One example is shown in FIG. 8b. The results of this primary screen with all 10 donors are summarized in Table 4.

TABLE 4
Summary of primary screen

| | | Reacting | Reacting | |
|---|---|---|---|---|
| | | with | with | Defining the |
| Donor | | horizontal | vertical | following peptides |
| number | HLA type | mixtures | mixtures | for retesting |
| 9936 | A2/24, B44/ | 19 | 36, 37, 38, 39 | 490, 491, 492, 493 |
| | 41 | | | |
| 10511 | A2/28, B16/ | 2, 10, 19, | 31, 33, 34, 35, | 37, 39-41, 43, 44, 46-48; |
| | 40 | 20, 21 | 37, 38, 40, 41, | 256, 258-260, |
| | | | 42 | 262, 263, 265-267; |
| | | | | 485, 487-489, |
| | | | | 491, 492, 494-496; |
| | | | | 506, 508-510, |
| | | | | 512, 513, 515-517; |
| | | | | 527, 529-531, |
| | | | | 533, 534; 536-538 |
| 10632 | A2/28, B12/ | 19 | 29-32; 34-39 | 483-486; 488-493 |
| | 27 | | | |
| 10687 | A2/11, B7/13 | 10, 11, 17, 18, | 22, 23; 33-42 | 247, 248, 258-267; |
| | | 19 | | 268, 269, 279-288; |
| | | | | 394, 395, 405-414; |
| | | | | 415, 416, 426, 427, 468-475; |
| | | | | 476, 477, 487-496 |
| 10689 | A2/25, B13/ | 8, 19 | 22, 23; 28, 29, | 205, 206; 211-214, |
| | 18 | | 30, 36-41 | 219-224; |
| | | | | 476, 477; 482-484; |
| | | | | 490-495 |
| 10736 | A2/3, B15/35 | 19 | 35-38 | 489-492 |
| 10764 | A2/24, B7/27 | 10, 17, 18, 20 | 22; 28-31; 36-42 | 247, 253-256, 261-267; |
| | | | | 394, 400-403, |
| | | | | 408-414; |
| | | | | 415, 421-424; |
| | | | | 469-475; |
| | | | | 497, 503-506; |
| | | | | 511-517 |
| 10788 | A2/29, B44/ | 2, 3, 4, 14-16, | 22-42 | 28-32, 36-48; 49-53, |
| | 60 | 17-19, | | 57-105; 331-335, |
| | | 21 | | 339-351; 352-356, |
| | | | | 360-372; 373-377; |
| | | | | 381-393; 394-398, |
| | | | | 402-414; 415-419, |
| | | | | 423-427; 468-475; |
| | | | | 476-480, 484-496; |
| | | | | 518-522; 526-538 |
| 10791 | A2/3, B7/27 | 11, 12, 19 | 32-42 | 278-287; 299-308; |
| | | | | 486-495 |

In a secondary screen all individual peptides corresponding to crossover points of positive row and column pools were retested. PBMC of the same donors were in this case incubated with the individual peptides at a final concentration of 20 Mg/ml for 20 hrs. Again a human IFN-gamma Elispot assay was used as a read out for T cell reactivation.

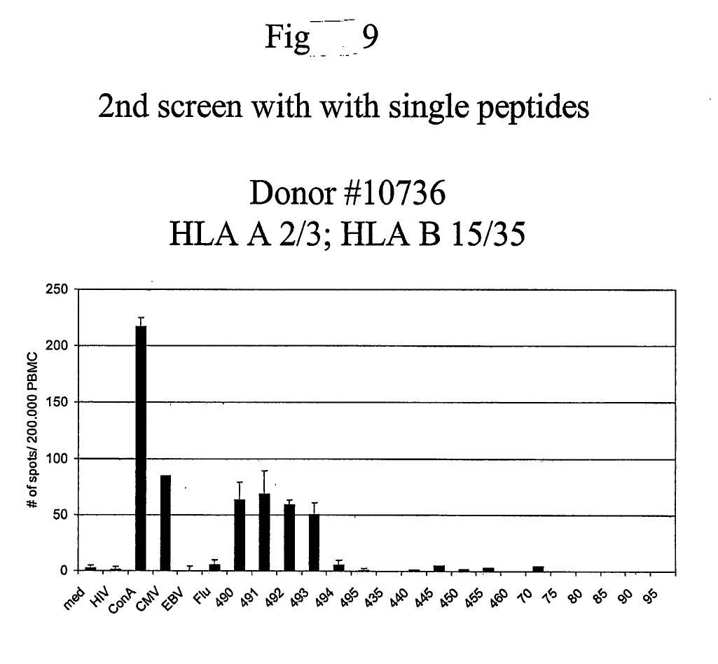

FIG. 9 shows the result from donor 10736, who had been identified in the primary screen to be an inividual with a highly focused T cell response (FIG. 8a). Retesting with individual peptides no. 489-495 confirmed reactivity of peptides 490-492 and in addition also identified peptide 493 as inducers of IFN-gamma secretion. The extent of the response to the 15mer peptides was again comparable to that of the 9-mer minimal epitope. A random selection of peptides corresponding to crossover points of negative row and column mixtures in the primary screen were included as negative controls; none did induce any IFN-gamma secretion (FIG. 9).

The complete results of this secondary screen and all epitopes defined by it are summarized in Table 5 (epitopes already described in the literature) and Table 6 (novel epitopes identified through the approach according to the present invention).

Among the already known epitopes, strikingly, the well-characterized CD8-restricted 9-mer minimal epitope (NLVPMVATV (SEQ ID NO:573), pos. 495-503) was recognized in 9 out of 10 donors, proving that it is one of the most frequently recognized HLA-A*0201 restricted epitopes in the CMV pp65 antigen. Among the 10 HLA A2 donors 2 also expressed HLA-B7. From the literature 2 HLA-B7 restricted epitopes are known: pp65 265-274 and pp65 417-426. Both were found in both HLA B7 positive donors and especially the pp65 417-426 epitope seemed to induce even more IFN-γ secretion than the HLA-A*0201 restricted pp65 495-503 epitope. This data are in agreement with the observation, that T cell responses against the CMV pp65 antigen is strongly linked to expression of HLA-A2 and/or HLA-B7 epitopes (Eur. J. Immunol., 2000, 30, 2531-2539. Beside these so-called “immunodominant” epitopes most other described epitopes within pp65 were re-discovered (Table 5). This demonstrates, that the present approach is both very sensitive and comprehensive and thus suitable for detection of both immunodominant and subdominant epitopes.

TABLE 5a
Comparison of found epitopes with literature known class I epitopes

| | Peptide | | | | |
|---|---|---|---|---|---|
| Peptides reactive in | as described | | Described HLA | | Frequency in |
| our screen | in literature | Described as | restriction | literature | screen¹⁾ |
| 0 | 14-22 | T cell epitope | A0201 | Solache et al., 1999 | 0/9 |
| # 10788: | 120-128 | T cell epitope | A0201 | Solache et al., 1999 | 1/9 |
| 117, 118, 119, 120 | | | | | |
| All except 10764: | 495-503 | T cell epitope | A0201 | Wills et al., 1996 & Weekes et al., | 8/9 |
| 489-493 | | | | 1999a | |
| #10764: | 522-530 | predicted | A0201 | Solache et al., 1999 | 1/9 |
| 522 | 517-531 | T cell response | | Kern et al., 1999 | |
| #10788: | 110-118 | Binding | A0201 | Solache et al., 1999 | 1/9 |
| 108 | 109-123 | T cell response | | Kern et al., 1999 | |
| #10687: | 286-295 | Binding | A0201 | Solache et al., 1999 | 2/9 |
| 282, 284 | 289-303 | T cell response | | Kern et al., 1999 | |
| # 10791: | | | | | |
| 282, 283, 284?, 285, | | | | | |
| 287 | | | | | |
| #10511: | 519-527 | Binding | A0201 | Solache et al., 1999 | 2/9 |
| 516?, 517 | 517-531 | T cell response | | Kern et al., 1999 | |
| # 10764 | | | | | |
| 513-517 | | | | | |
| 0 | 316-338 | T cell epitope | A0201 | Bitmansour et al., 2001 | 0/9 |

Found: 6 out of 8 literature known epitopes in 9 HLA A2 positive donors

| | | | | | |
|---|---|---|---|---|---|
| 0 | 353-375 | T cell epitope | A1 | Retiere et al., 2000 | 0/0 |

Found: 0 out of 1 literature known epitopes in 0 HLA A1 positive donors

| | | | | | |
|---|---|---|---|---|---|
| 0 | 113-121 | T cell epitope | A24 | Retiere et al., 2001 | 0/2 |
| 0 | 328-337 | T cell epitope | A24 | Kuzushima et al., 2001 | 0/2 |

Found: 0 out of 2 literature known epitopes in 2 HLA A24 positive donors

| | | | | | |
|---|---|---|---|---|---|
| Possibly recognized | 512-520 | T cell epitope | B12 | Wills et al., 1996& Weekes et al., | 1/1 |
| in #10632 | | | | 1999a | |

Found: 1 out of 1 literature known epitopes in1 HLA B12 positive donor

| | | | | | |
|---|---|---|---|---|---|
| 0 | 123-131 | T cell epitope | B35 | Gavin et al., 1993 & Wills et al., | 0/1 |
| | | | | 1996 | |
| 0 | 187-195 | T cell epitope | B35 | McLaughlin-Taylor et al., 1994 | 0/1 |
| # 10687(B7 and not | 397-411 | T cell epitope | B35 | Wills et al., 1996& Weekes et al., | 1/7 |
| B35): | | | | 1999a | |
| 394, 395 | | | | | |

Found: 0 out of 3 literature known epitopes in 1 B35 positive donor; 1 found in HLA B35 negative donor

| | | | | | |
|---|---|---|---|---|---|
| #10687: | 265-274 | T cell epitope | B7 | Weekes et al., 1999b | 2/2 |
| 260-265 | | | | | |
| #10764: | | | | | |
| 261-264 | | | | | |
| #10687: | 417-426 | T cell epitope | B7 | Weekes et al., 1999a | 2/2 |
| 414, 415, 416 | | | | | |
| #10764: | | | | | |
| 413, 414, 415 | | | | | |

Found: 2 out of 2 literature known epitopes in 2 HLA B7 positive donors
¹⁾Number of refinding/number of donors' expressing the respective HLA type

TABLE 5b
Comparison of found epitopes with literature known class II epitopes

| | Peptide | | | | |
|---|---|---|---|---|---|
| Peptides | as | | Described | | |
| reactive in | described | | HLA | | Frequency |
| our screen | in literature | Described as | restriction | literature | in screen¹⁾ |
| 0 | 34-56 | T cell epitope | DQ0602 | Bitmansour et al., 2001 | 0 |
| 0 | 364-386 | T cell epitope | D1302 | Bitmansour et al., 2001 | 0 |
| #10788: | 361-376 | T cell epitope | DR11 | Khattab et al., 1997 | 1 |
| 361 | | | | | |
| #10511 | 509-523 | T cell epitope | DR3 | Khattab et al., 1997& Bitmansour | 1 |
| 509, 510 | | | | et al., 2001 | |
| 0 | 144-166 | T cell epitope | DR1401 | Bitmansour et al., 2001 | 0 |
| 0 | 177-191 | T cell epitope | class II | Bitmansour et al., 2001 | 0 |
| Possibly: | 285-299 | T cell epitope | class II | Bitmansour et al., 2001 | 2 |
| #10687: | | | | | |
| 284 | | | | | |
| #10791: | | | | | |
| 285 | | | | | |
| 0 | 417-431 | T cell epitope | class II | Bitmansour et al., 2001 | 0 |
| Possibly: | 489-503 | T cell epitope | class II | Bitmansour et al., 2001 | 2 |
| 10511: | | | | | |
| 487, 489 | | | | | |
| 10687: | | | | | |
| 487, 488, 489 | | | | | |
| 0 | 205-219 | T cell epitope | ? | Kern et al., 1999 | 0 |
| Possibly | 293-307 | T cell epitope | ? | Kern et al., 1999 | 1 |
| #10687: | | | | | |
| 287, 288 | | | | | |

¹⁾no information on the MHC class II phenotype of the donors' is available

Table 6 lists all novel T cell epitopes which have been identified through the approach according to the present invention as described in Examples I and II.

The first group of epitopes (peptides 262, 394, 395, 411, 415, 416, 417) comprises sequences, which have already been described in the literature as T cell epitopes, albeit with a different HLA restriction than found here. The present results show that these epitopes are also recognized by T cells in a different HLA context than known before. This finding is new and expands the usefulness of these peptides e.g. as part of vaccines suitable for individuals expressing these particular HLAs.

The second group of epitopes (peptides 213, 214 and 216) have already been described in the literature as binding to HLA-A*0201 molecules, however before our work there was no data proofing that these peptides can activate T cells. Binding of a peptide is a pre-requisite for activation of/recognition by T cells but does not guarantee it. First, not all possible peptide sequences within an antigen are generated equally well or at all during antigen processing and presentation in vivo. Thus a synthetic peptide binding to HLA is not necessarily a naturally occurring epitope. Even if T cells can be primed against such peptides by active vaccination, they are not useful against the pathogen, because an infected cell does not display these peptides on its surface. Second, even if a binding peptide corresponds to a naturally occurring epitope this does not guarantee, that it can induce functional T cells. Instead certain peptides can act as antagonists and anergize T cells. The present data show for the first time that these peptides can induce functional, IFN-gamma secreting T cells in humans as a consequence of viral infection. This finding is new and provides a rational for including these peptides in vaccines against CMV.

The third group of epitopes (peptides 211, 476, 477, 479, 421, 422, 423, 424, 469, 470, 503, 506) have so far not been described at all. Since the present data data show that these peptides can induce functional, IFN-gamma secreting T cells in humans as a consequence of viral infection, they represent by themselves or contain within their sequence novel T cell epitopes. These are especially useful for inclusion in vaccines against CMV.

Since the screening described in this Example uses whole PBMCs, the complete T cell response to any of the 15mers was analyzed. This response can be restricted to any of the HLA-alleles expressed by the respective donors. These include, in each case at least two possible alleles of, HLA-A, -B, -C (class I) and HLA-DR, -DP, -DQ (class II). For further characterizing the novel epitopes provided herewith, one may define the exact HLA restriction of these epitopes and the minimal epitopes within the sequences recognized by T cells. Both can be done by a variety of well-established approaches known to the one skilled in the art (Current Protocols in Immunology, John Wiley & Sons, Inc.).

First, publicly available programs can be used to predict T cell epitopes on the basis of binding motifs. These include for instance: http://bimas.dcrt.nih.gov/molbio/hla_bind/ (Parker et al. 1994), http://134.2.96.221/scripts/MHCServer.dll/home.htm (Rammensee at al. 1999), http://mypage.ihost.com/usinet.hamme76/ (Sturniolo et al. 1999). The latter prediction algorithm offers the possibility to identify promiscuous T helper-epitopes, i.e. peptides that bind to several HLA class II molecules. The respective HLA molecules, which were predicted with highest probability, are listed in Table 4. These prediction can be verified by testing of binding of the peptide to the respective HLA. Sequences, which were predicted to be possible class II epitopes, were tested for their binding to DRB1*0101, *0401 and *0404 molecules (see example III). The peptides 421, 469 and 470 (spanning aa 421-438, 469-484 and 470-485) were found to bind to DRB1*0404 (see Table 5).

A way of quickly discerning whether the response towards a peptide is class I or class II restricted is to repeat the ELIspot assay with pure CD4+ or CD8+ T cell effector populations. This can for instance be achieved by isolation of the respective subset by means of magnetic cell sorting. Pure CD8+ T cells can also be tested in ELIspot assays together with artificial antigen-presenting-cells, expressing only one HLA molecule of interest. One example are HLA-A*0201 positive T2 cells (174CEM.T2, Nijman et al., 1993). Alternatively, one can use ELIspot assays with whole PBMCs in the presence of monoclonal antibodies specifically blocking either the CD4+ or CD8+ T cell sub-population. Exact HLA restriction can be determined in a similar way, using blocking monoclonal antibodies specific for a certain allele. For example the response against an HLA-A24 restricted epitope can be specifically blocked by addition of an HLA-A24 specific monoclonal antibody.

For definition of the minimal epitopes within the peptide sequences recognized by T cells, one can e.g. synthesize series of overlapping and truncated peptides (e.g. 8-, 9-, 10-mers and re-test these individually.

### Example V

### Confirmation of Novel HLA-A*0201 Epitopes by T2-ELIspot

In order to confirm predicted HLA-A2 epitopes within positive 15mers, 9- or 10-mer peptides were synthesized and tested individually. ELIspot assays were performed with T2 cells as antigen-presenting cells and isolated CD8+ T-cells from several donors. In this setting only optimal-length epitopes binding from the outside to HLA-A*0201 can trigger IFN-γ secretion. Five novel HLA-A*0201 epitopes could be confirmed by this approach (Table 6). The assay was carried out as described (Herr 1997). Briefly, T2 cells (Nijman 1993) were grown in ELIspot medium (see M&M section) and adjusted to 4×10⁵/ml 3 days prior use. After washing 2 times (PBS; 1% human albumin from SIGMA), cells were seeded at 2.5×10⁶/ml in ELIspot medium and 10 μg/ml peptide was added for over night culture. The next day T2 cells were washed once with ELIspot medium and 100 μl (1×10⁶ T2 cells/ml) were seeded into coated and blocked ELIspot plates (see above), supplemented with 20 μg/ml peptide. For isolation of CD8+ lymphocytes 1×10⁷ PBMNC were mixed with 20 μl anti human CD8+ Microbeads (Miltenyi Biotec, Bergisch Gladbach, Germany) in a final volume of 100 μl and incubated for 15 min on ice. Afterwards cells were washed with a 10 times excess of MACS buffer and CD8+ cells were isolated on magnetic Minicolumns (Miltenyi). 100 μl of CD8+ lymphocytes adjusted to 1×10⁶ c/ml were added per well. After a culture period of 20 h cells were removed by washing 4 times with PBS, 0.05% Tween 20 and the assay was developed as described in the M&M section.

TABLE 6

CD8+ T-cell responses against HLA-A*0201
restricted epitopes of CMV pp65 assessed by T2
ELlspot assays for IFN-γ.

| Donor ¹⁾ | | | | | |
|---|---|---|---|---|---|
| Epitope ²⁾ | 10511 | 10632 | 10687 | 10689 | 10788 |
| ILKEPVHGV | 17( [symbol] 4)³⁾ | 16( [symbol] 3) | 25( [symbol] 7) | 77( [symbol] 2) | 34( [symbol] 2) |
| (SEQ ID NO:574) | | | | | |
| (HIV, negative control) | | | | | |
| NLVPMVATV 495-503 | < | 165( [symbol] 10) | 193( [symbol] 1) | 189( [symbol] 10) | 172( [symbol] 2) |
| (SEQ ID NO:573) | | | | | |
| (CMV, positive control) | | | | | |
| RLLQTGIHV 40-48 | < | < | < | < | 67( [symbol] 9) |
| (SEQ ID NO:7) | | | | | |
| VIGDQYVKV 218-226 | < | 31( [symbol] 1) | 112( [symbol] 5) | < | 100( [symbol] 8) |
| (SEQ ID NO:8) | | | | | |
| YLESFCEDV 227-235 | < | < | < | < | < |
| (SEQ ID NO:9) | | | | | |
| AMAGASTSA 425-433 < | < | 96( [symbol] 5) | < | 104( [symbol] 8) | |
| (SEQ ID NO:4) | | | | | |
| FTWPPWQAGI 482-491 | < | < | < | < | 70( [symbol] |
| (SEQ ID NO:3) | | | | | |
| KYQEFFWDA 509-517 | < | 32( [symbol] 1) | < | < | 72( [symbol] 22) |
| (SEQ ID NO:6) | | | | | |
| RIFAELEGV 522-530 | < | < | 92( [symbol] 8) | < | 71( [symbol] 18) |
| (SEQ ID NO:575) | | | | | |

¹⁾new blood draws 3-6 months apart from primary matrix screen
²⁾new epitopes shown boldface
³⁾spot numbers per 100,000 CD8+ T-cells; <: below background (spots with HIV negative control peptide) plus 2 standard deviations.

### Example VI

### Confirmation of Novel Class II (HLA-DR4) Epitopes Identified by the Epitope Capture Method Using Human PBMC

Among others, 15mers 55-61 were identified by the epitope capture method as strong binders of soluble HLA-DRB1*0401 (FIG. 7a, 7b). Binding of individual overlapping 15mers 55-61 identifies the amino acid sequence YTPDSTPCH (SEQ ID NO:576) as core binding region of LILVSQYTPDSTPCHRGDNQL (SEQ ID NO:577) which may contain several versions of a class II epitope. It is well known that class II epitopes have ends of variable length protruding the MHC binding grove. Interestingly, donor 10788 showed strong T-cell responses against 15mers 57 and 59 (FIG. 10) measured by IFN-γ ELIspot as described in Example IV. This confirms that LILVSQYTPDSTPCHRGDNQL (SEQ ID NO:577) represents or contains at least one HTL epitope binding to at least HLA-DRB1*0401 originally discovered by the epitope capture method of the present invention.

FIG. 10: PBMC from subject 10788 were applied for IFN-γ ELIspot with CMVpp65 15mers 57, 59 and controls (med: no peptide, HIV: irrelevant HIV-derived peptide, ConA: polyclonal stimulation.

As a further example 15mers 469 and 470 identified by the epitope capture method of the present invention as weakly binding to DRB1*0404 (Tab. 3) were tested for reactivity with human PBMC. To distinguish between CD8-positive CTL mediated or CD4-positive HTL mediated reactivity, intracellular cytokine staining combined with staining for surface markers was performed: after thawing, PBMNC were kept overnight (37° C., 5% CO₂) in RPMI1640 with 10% human serum type AB (BioWhittaker). Next day aliquots of 2 million PBMNC were incubated with peptide (80 μg/ml) or Concanavalin A (SIGMA). After 1 h, 10 μg/ml brefeldin A (SIGMA) were added and incubation was continued for 5 hours. Surface staining for CD4 (phycoerythrin), CD8 (cychrome), CD69 (allophycocyanine) was done with antibodies labelled as indicated (Pharmingen, San Diego, Calif., USA). After fixation with 1% para-formaldehyde in PBS, cells were permeabilized by incubating for 15 min in 0.5% BSA/0.1% Na-azide/0.1% saponin in PBS. Intracellular cytokines were stained with anti-IFN-γ (FITC) antibody 4S.B3 (Pharmingen). Samples (100,000 events in the lymphocyte gate) were read on a FACScalibur (Becton Dickinson).

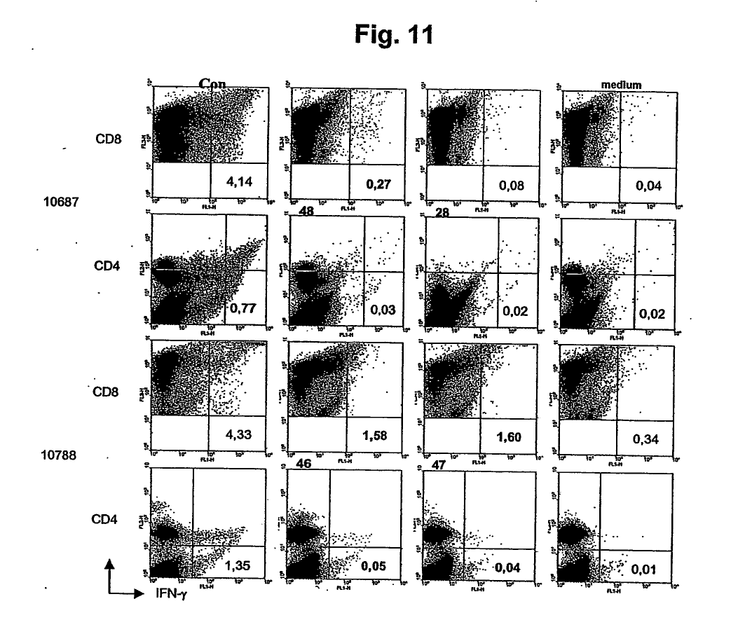

15mers 469 and 470 covering DEDSDNEIHNPAVFTW₄₆₉₋₄₈₄ (SEQ ID NO:578) contain both A*0201 and class II binding motifs and bind to soluble DRB1*0404 (Tab. 3). Intracellular cytokine staining confirmed significant IFN-γ secretion in both the CD8 and CD4 positive T-cell compartments for donor 10788 (FIG. 11, lower two panels). Conversely, donor 10687 showed only CD8 mediated IFN-γ secretion against 15mer 489 AGILARNLVPMVATV₄₈₉₋₅₀₃ (SEQ ID NO:514) indicating that in this case only the HLA-A2 epitope NLVPMVATV₄₉₅₋₅₀₃ (SEQ ID NO:573) was targeted. These results confirm that peptide DEDSDNEIHNPAVFTW₄₆₉₋₄₈₄ (SEQ ID NO:578) contains both class I and class II restricted epitopes.

FIG. 11: Confirmation of simultaneous CD4+ and CD8+ T-cell responses against CMVpp65 15mers 469, 470 by intracellular IFN-γ staining. PBMC from 2 donors (10687, 10788) were stimulated with either ConA as positive control (1st column), 15mers containing both putative class I and class II epitopes (columns 2 and 3) or medium as negative control (right column). Cells were stained for intracellular IFN-γ (x-axis) or surface the T-cell differentiation markers CD4 or CD8 (y-axis). Percentage in upper-right quadrant is indicated, numbers significant over background are shown boldface.

### Example VII

### Confirmation of Novel class II (HLA-DRB1*0401) Epitopes Identified by the Epitope Capture Method Using HLA-DR4 Transgenic Mice

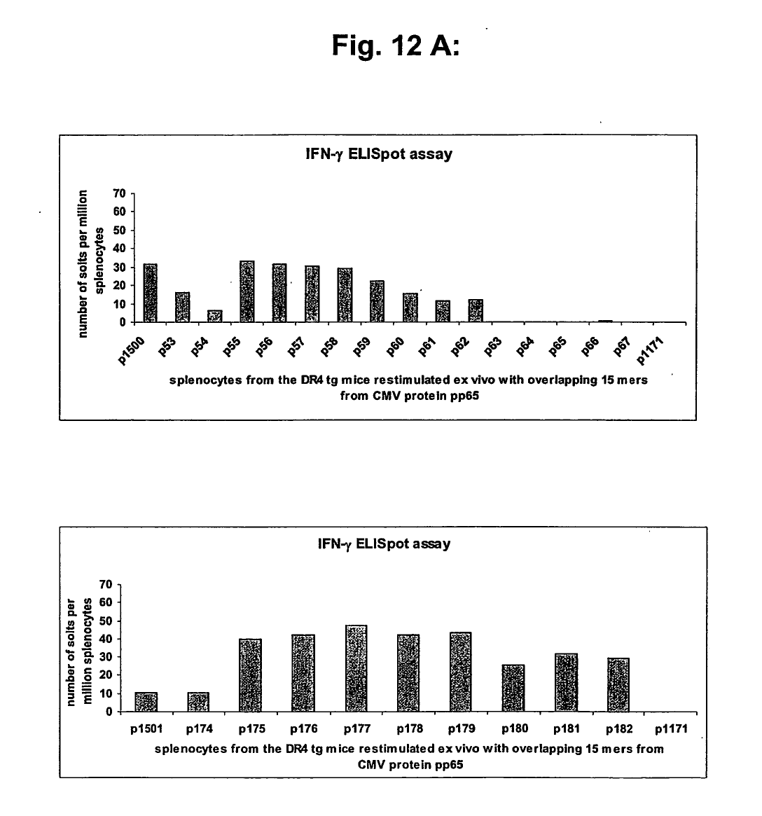

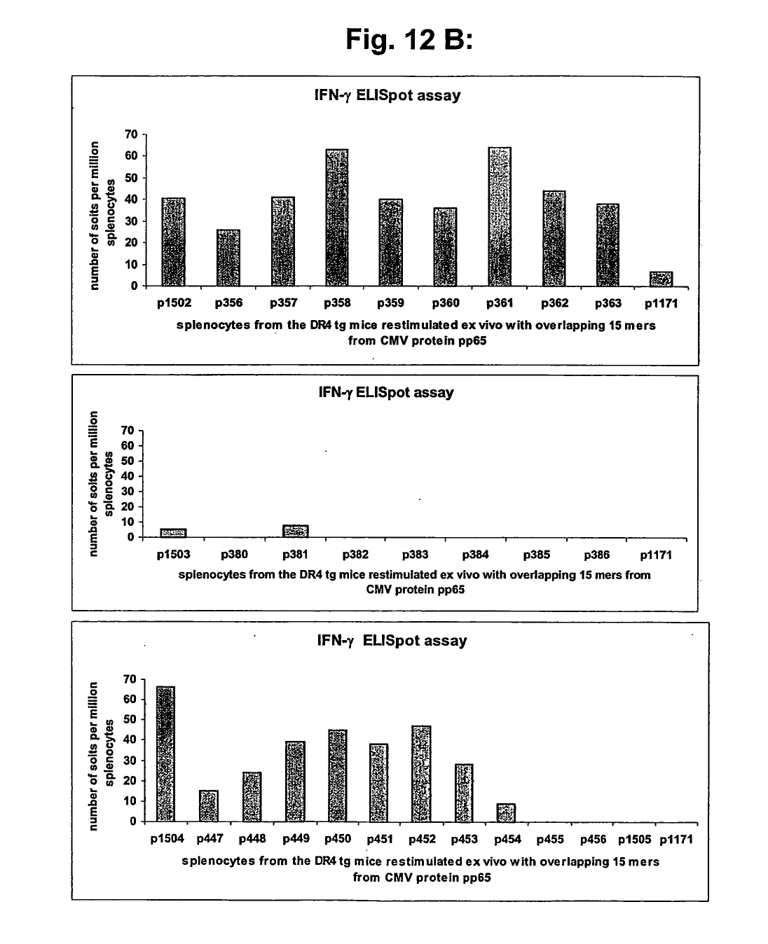

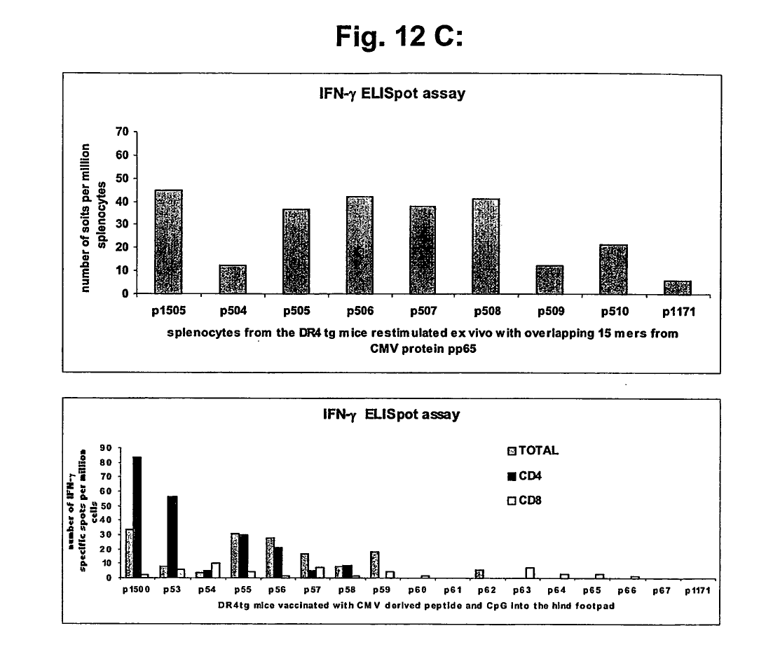

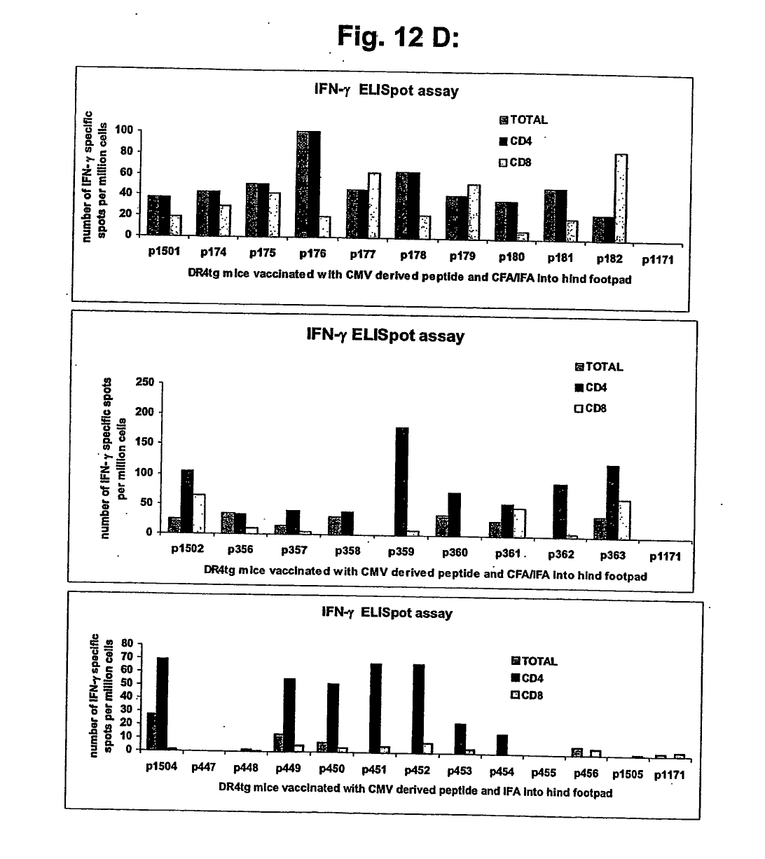

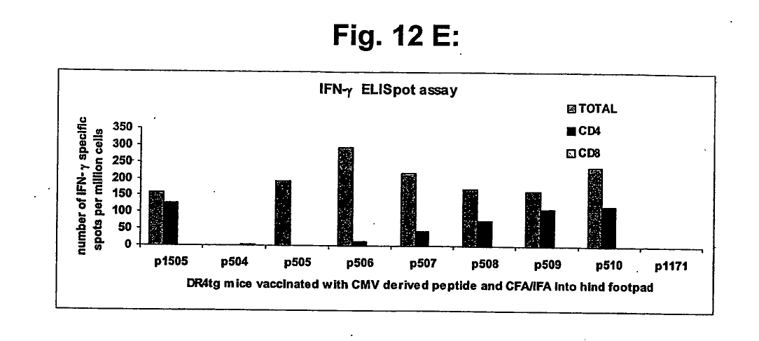

For those, DRB1*0401 binding, peptides where T cell reactivity was not demonstrated by testing CMV seropositive individuals, experiments in HLA-DR4 transgenic mice, expressing DRB1*0401 molecules, were performed. The longer peptides (no. 1500-1505), covering all candidate epitopes binding to DRB1*0401 molecules, were synthesized (see Table 7) and injected into the mice. One week after the last injection total murine splenocytes were re-stimulated ex vivo with the same peptides that were used for vaccination, as well as with overlapping 15-mers, representing corresponding longer peptides and an irrelevant, influenza hemaglutinin derived peptide (no. 1171) as a negative control. T cell reactivity was determined by INF-gamma ELISpot assay (FIG. 12) and as a result, five out of six longer peptides were shown to be immunogenic. Moreover, most of the 15-mers, representing longer peptides and showing affinity to DRB1*0401 molecules, were also verified to re-activate ex vivo T cells from DRB1*0401-transgenic mice. One peptide (no. 1503) did not induce immune response at least under the immunization conditions used in these studies.

For the fine epitope mapping and in vivo testing of CD4+ specificity of immunogenic peptides, mice were vaccinated with the longer peptide and splenocytes were separated into CD4+ and CD8+ T cell populations (92-94% purity for CD4+ fraction) to be tested for IFN-γ production as described above. FIG. 12 shows the results of such ELlSpot assay. In all cases the T cell response measured with splenocytes could be confirmed using CD4+ cells. Usually the CD8+ response was negligible. A simultaneous CD4+ and CD8+ response as seen for peptide 1502 could be due to an additional class I mouse epitope contained within the peptide sequence.

Table 7a summarizes all the data of peptide binding studies with soluble DR molecules and mouse experiments. The good correlation between peptide affinities to DRB1*0401 molecules and specific stimulation of CD4+ cells in HLA-DR4 transgenic mice was observed. For instance, peptides no. 1500-1502 and 1504 showed similar results in both approaches. Peptide no. 1505 that was not so good in binding assay evoked the highest frequency of T cells generated in mice after peptide injection. Peptide no. 1503 that had weak affinity to DRB1*0401 molecules in vitro was not immunogenic in mice injected either with CpG1668 or with CFA/IFA. Here should be also mentioned that some of peptides showed variable immunogenicity and specific CD4+ frequency depending on the adjuvant used for the injection. Based on the results of mouse ELISpot assay with enriched fraction of CD4+ cells and peptide binding assay with soluble DRB1*0401 molecules, epitope core sequences recognized by DRB1*0401 carrying T cell (see Table 7 and Table 7a) can be determined. Four of them fit well to the predicted by TEPITOPE algorithm sequences, except one for peptide no. 1501 which is not predicted to bind DRB1*0401 molecules.

TABLE 7

Verification of DRB1*0401 ligands, as candidate
epitopes, in transgenic mice.
Proposed DRB1*0401 core binding motifs are highlighted.

| | |
|---|---|
| Binding to DRB1* | DRB1*0401 mice |

| | | | 040 | 040 | 010 | 070 | | total | | Comment, |
|---|---|---|---|---|---|---|---|---|---|---|
| No. | Peptide sequence | AA | 1 | 4 | 1 | 1 | 1101 | SC | CD4+ | reference |
| 1500 | PSLILVSQYTPDSTPCHRG | 53- | ++ | ++ | + | − | − | + | + | new epitope, |
| | DNQLQVQHTR | 81 | | | | | | | | human PBMC's |
| | SEQ ID No: 579) | | | | | | | | | in this study |
| 1501 | QWKEPDVYYTSAFVFPTKD | 174- | + | − | ++ | − | +/− | + | + | Bitmansour |
| | VALR | 196 | | | | | | | | et al., |
| | (SEQ ID NO:580) | | | | | | | | | CD4+clone |
| 1502 | LLQRGPQYSEHPTFTSQYR | 356 | + | − | − | − | ++ | + | ++ | Khattab |
| | IQG | - | | | | | | | | et al., |
| | (SEQ ID No:581) | 377 | | | | | | | | CD4 + |
| | | | | | | | | | | clone/DR11 |
| 1503 | YRHTWDRHDEGAAQG | 381 | +/− | − | − | − | − | − | − | not |
| | DDDVW | - | | | | | | | | immunogenic |
| | (SEQ ID No:582) | 400 | | | | | | | | |
| 1504 | TSGVMTRGRLKAESTVAPE | 447 | + | +++ | + | − | + | + | ++ | new epitope |
| | EDTDE | - | | | | | | | | |
| | (SEQ ID NO:583) | 470 | | | | | | | | |
| 1505 | QGQNLKYQEFFWDANDIYR | 504 | +/− | − | − | − | − | ++ | ++ | Khattab et |
| | IF | - | | | | | | | | al., CD4+ |
| | (SEQ ID No:584) | 524 | | | | | | | | clone/DR3 |

“−” no binding/T cell response; “+/−” weak binding/T cell response; “+” intermediate binding/T cell response; “++”strong binding/T cell response

In conclusion, most of the peptides discovered as ligands for soluble DRB1*0401 molecules in the present “reverse immunological” approach were confirmed to elicit specific CD4+ response in DRB1*0401-transgenic mice. Thus, five out of six epitope candidate are real DRB1*0401 epitopes. Additionally, they are promiscuous (see Table 7 and Table 7a). Three of them (no. 1501, 1502 and 1503) have been described in the literature earlier but not as DRB1*0401 specific epitopes. Two DRB1*0401 binders (no. 57 and 59) and three peptides binding to DRB1*0404 (no. 421, 469 and 470) were also found to induce INF-gamma production in PBMC's from CMV seropositive donors (see Example IV and VI). All together, it indicates that the present Epitope Capture Approach can identify true T cell epitopes.

TABLE 7a

Summary of identification of human class II epitopes using
soluble DRBl*0401 molecule molecule and HLA DR4-transgenic mice
Proposed DRB1*0401 core binding motifs are highlighted.

| | | |
|---|---|---|
| Pep | Binding to DRB1 | DRB1*0401 mice |

| | | | | | | | | | |
|---|---|---|---|---|---|---|---|---|---|
| No. | Peptide sequence | *0401 | *0404 | *0101 | *0701 | 1101 | total SC | CD4+ | Comments, |

| | | | | | | | | | |
|---|---|---|---|---|---|---|---|---|---|
| 1500 | PSLILVSQYTPDSTPCHRGDNQLQ | ++ | ++ | + | − | − | + | + | AA 53-81 |
| | VQHTR (SEQ ID No:579) | identifie | newly | | | | | | |
| | | | | | | | | | identified |
| 54 | SLILVSQYTPOSTPC | − | | | | | − | +/− | |
| | (SEQ ID NO:79) | | | | | | | | |
| 55 | LILVSQYTPDSTPCH | + | | | | | ++ | + | |
| | (SEQ ID NO:80) | | | | | | | | |
| 56 | ILVSQYTPDSTPCHR | ++ | | | | | ++ | + | |
| | (SEQ ID NO:81) | | | | | | | | |
| 57 | LVSQYTPDSTPCHRG | ++ | − | − | | | ++ | + | |
| | (SEQ ID NO:82) | | | | | | | | |
| 58 | VSQYTPDSTPCHRGD | ++ | − | + | − | − | ++ | + | |
| | (SEQ ID NO:83) | | | | | | | | |
| 59 | SQYTPDSTPCHRGDN | ++ | | | | | ++ | +/− | |
| | (SEQ ID NO:84) | | | | | | | | |
| 60 | QYTPDSTPCHRGDNQ | ++ | | | | | ++ | +/− | |
| | (SEQ ID NO:85) | | | | | | | | |
| 61 | YTPDSTPCHRGDNQL | + | | | | | + | +/− | |
| | (SEQ ID NO:86) | | | | | | | | |
| 62 | TPDSTPCHRGDNQLQ | − | | | | | − | − | |
| | (SEQ ID NO:87) | | | | | | | | |
| 63 | PDSTPCHRGDNQLQV | − | | | | | − | − | |
| | (SEQ ID NO:88) | | | | | | | | |
| 64 | DSTPCHRGDNQLQVQ | + | − | − | | | | | |
| | (SEQ ID NO:89) | | | | | | | | |
| 65 | STPCHRGDNQLQVQH | − | | | | | − | − | |
| | (SEQ ID NO:90) | | | | | | | | |
| 1501 | QWKEPDVYYTSAFVFPTKDVALR | + | − | ++ | − | + | + | + | AA |
| | (SEQ ID ND:580) | | | | | | | | 174 -196, |
| | | | | | | | | | known |
| | | | | | | | | | class II |
| 174 | QWKEPDVYYTSAFVF | − | | | | | +/− | +/− | |
| | (SEQ ID NO:199) | | | | | | | | |
| 175 | WKEPDVYYTSAFVFP | − | | | | | + | + | |
| | (SEQ ID NO:200) | | | | | | | | |
| 176 | KEPDVYYTSAFVFPT | +/− | | | | | + | + | |
| | (SEQ ID NO:201) | | | | | | | | |
| 177 | EPDVYYTSAFVFPTK | + | − | ++ | − | + | + | + | |
| | (SEQ ID NO:202) | | | | | | | | |
| 178 | PDVYYTSAFVFPTKD | ++ | | | | | + | + | |
| | (SEQ ID NO:203) | | | | | | | | |
| 179 | DVYYTSAPVFPTKDV | + | | | | | + | + | |
| | (SEQ ID NO:204) | | | | | | | | |
| 180 | VYYTSAFVFPTKDVA | +/− | | | | | + | +/− | |
| | (SEQ ID NO:205) | | | | | | | | |
| 181 | YYTSAFVFPTKDVAL | − | | | | | + | +/− | |
| | (SEQ ID NO:206) | | | | | | | | |
| 182 | YTSAFVFPTKDVALR | | | | | | +/− | +/− | |
| | (SEQ ID NO:207) | | | | | | | | |
| 1502 | LLQRGPQYSEHPTFTSQYRIQG | + | − | − | − | ++ | ++ | ++ | AA |
| | (SEQ ID No:581) | | | | | | | | 356-377 |
| | | | | | | | | | known DR |
| 356 | LLQRGPQYSEHPTFT | | | | | | +/− | +/− | 11 |
| | (SEQ ID ND:381) | | | | | | | | |
| 357 | LQRGPQYSEHPTFTS | − | | | | | + | +/− | |
| | (SEQ ID NO:382) | | | | | | | | |
| 358 | QRGPQYSEHPTPTsQ | + | | | | | ++ | +/− | |
| | (SEQ ID NO:383) | | | | | | | | |
| 359 | RGPQYSEHPTFTSQY | +/− | − | − | − | +/− | ++ | ++ | |
| | (SEQ ID NO:384) | | | | | | | | |
| 360 | GPQYSEHPTFTSQYR | +/− | | | | | ++ | ++ | |
| | (SEQ ID NO:385) | | | | | | | | |
| 361 | PQYSEHPTFTSQYRI | +/− | | | | | + | ++ | |
| | (SEQ ID NO:386) | | | | | | | | |
| 362 | QYSEHPTFTSQYRIQ | − | | | | | + | ++ | |
| | (SEQ ID NO:387) | | | | | | | | |
| 363 | YSEHPTFTSQYRIQG | | | | | | + | ++ | |
| | (SEQ ID NO:388) | | | | | | | | |
| 1503 | YRHTWDRHDEGAAQGDDDVW | +/− | − | − | − | − | − | − | AA |
| | (SEQ ID NO:582) | | | | | | | | 381-400 |
| | | | | | | | | | not |
| | | | | | | | | | immunogenic |
| 380 | EYRHTWDRHDEGAAQ | − | | | | | − | − | |
| | (SEQ ID NO:405) | | | | | | | | |
| 381 | YRHTWDRHDEGAAQG | − | | | | | − | − | |
| | (SEQ ID No:406) | | | | | | | | |
| 382 | RHTWDRHDEGAAQGD | − | | | | | − | − | |
| | (SEQ ID No:407) | | | | | | | | |
| 383 | HTWDRHDEGAAQGDD | +/− | | | | | − | − | |
| | (SEQ ID No:408) | | | | | | | | |
| 384 | TWDRHDEGAAQGDDD | +/− | − | − | − | − | − | − | |
| | (SEQ ID NO:409) | | | | | | | | |
| 385 | WDRHDEGAAQGDDDV | − | | | | | − | − | |
| | (SEQ ID 140:410) | | | | | | | | |
| 1504 | TSGVMTRGRLKAESTVAPEEDTDE | + | ++ | + | − | + | + | ++ | AA 447- |
| | (SEQ ID NO:583) | 470, | | | | | | | |
| | | | | | | | | | newly |
| | | | | | | | | | identified |
| 448 | SGVMTRGRLKAESTV | − | | | | | +/− | − | |
| | (SEQ ID NO:473) | | | | | | | | |
| 449 | GVMTRGRLKAESTVA | +/− | | | | | + | ++ | |
| | (SEQ ID NO:474) | | | | | | | | |
| 450 | VMTRGRLKAESTVAP | + | | | | | + | ++ | |
| | (SEQ ID NO:475) | | | | | | | | |
| 451 | MTRGRLKAESTVAPE | + | | | | | + | ++ | |
| | (SEQ ID NO:476) | | | | | | | | |
| 452 | TRGRLKAESTVAPEE | + | ++ | ++ | − | − | + | ++ | |
| | (SEQ ID 140:477) | | | | | | | | |
| 453 | RGRLKAESTVAPEED | + | | | | | +/− | +/− | |
| | (SEQ ID NO:478) | | | | | | | | |
| 454 | GRLKAESTVAPEEDT | + | | | | | +/− | +/− | |
| | (SEQ ID NO:479) | | | | | | | | |
| 455 | RLKAESTVAPEEDTD | + | | | | | +/− | +/− | |
| | (SEQ ID NO:480) | | | | | | | | |
| 456 | LKAESTVAPEEDTDE | | | | | | − | − | |
| | (SEQ ID NO:481) | | | | | | | | |
| 1505 | QGQNLKYQEFFWDANDIYRIF | + | − | − | − | − | ++ | ++ | AA 504-- |
| | (SEQ ID No:584) | | | | | | | | 524 |
| | | | | | | | | | known DR3 |
| 504 | QGQNLKYQEFFWDAN | − | | | | | − | − | |
| | (SEQ ID No:529) | | | | | | | | |
| 505 | GQNLKYQEFFWDAND | − | | | | | + | − | |
| | (SEQ ID No:530) | | | | | | | | |
| 506 | QNLKYQEFFWDANDI | − | | | | | ++ | +/− | |
| | (SEQ ID No:531) | | | | | | | | |
| 507 | NLKYQEFFWDANDIY | +/− | − | − | − | − | ++ | + | |
| | (SEQ ID No:532) | | | | | | | | |
| 508 | LKYQEFFWDANDIYR | +/− | | | | | ++ | ++ | |
| | (SEQ ID No:533) | | | | | | | | |
| 509 | KYQEFFWDANDIYRI | − | | | | | ++ | ++ | |
| | (SEQ ID No:534) | | | | | | | | |
| 510 | YQEFFWDANDIYRIF | − | | | | | ++ | ++ | |
| | (SEQ ID Wo:535) | | | | | | | | |

“−” no binding/T cell response; “+/−” weak binding/T cell response; “+” intermediate binding/T cell response; “++” strong binding/T cell response

### REFERENCES

1. Aichinger G et al., 1997. J. Biol. Chem. 272: 29127-29136.
2. Ausubel F M et al. (editors), 2001. Current Protocols in Molecular Biology, Volumes 1-4, John Wiley and Sons (publishers).
3. Bitmansour A D et al., 2001. J. Immunol. 167: 1151-1163.
4. Bonifacino J S et al. (editors), 2001. Current Protocols in Cell Biology, Volumes 1-2, John Wiley and Sons (publishers).
5. Britt W J et al, 1996. Chapter 77 “Cytomegalovirus”, Virology by Fields et al. (editors), Livincott-Raven Publishers
6. Cox A L et al., 1994. Science 264: 716-719
7. Coligan J E et al. (editors), 2001. Current Protocols in Immunology, Volumes 1-4, John Wiley and sons (publishers).
8. Drew W L et al, 1999. Curr Clin Top Infect Dis 19: 16-29
9. Field A K et al., 1999. Antivir Chem Chemother 10(5): 219-232
10. Fowler K B et al, 1992. N Engl J Med 326: 663-673
11. Gallot G. et al., 2001. J. Immunol. 167: 4196-4206.
12. Gavin M A et al., 1993. J. Immunol. 151: 3971-80
13. Gorga, J C et al., 1987. J. Biol. Chem. 262: 16087-16094.
14. Greenberg, P. D. et al, 1999. Science 285: 546.
15. Heemels, M T et al, 1995. Annu. Rev. Biochem. 64: 463-491
16. Kern F et al, 2000. Eur J Immun 30: 1676-1682
17. Kern F et al, 1999. J Virol 73(10): 8179-8184.
18. Khattab B A et al., 1997. J Med Virol 52: 68-76.
19. Klein, J, 1986. Natural History of the MHC, John Wiley and Sons (publishers)
20. Komanduri, K V et al., 1998. Nat. Med. 4: 953.
21. Kronenberg M et al., 1999. Immunol Today 20: 515-21.
22. Kuzushima K et al., 2001. Blood 98(6): 1872-1880.
23. Kwok W W et al., 2001. Trends Immunol 22(11): 583-588.
24. Lalvani A et al., 1997. J. Exp. Med. 186 (6): 859-865.
25. McLaughlin-Taylor E et al., 1994. J Med Virol 43: 103-110.
26. Maecker H T et al, 2001. J Immunol Methods 255: 27-40
27. Masuoka M et al., 2001. Viral Immunology 14(4): 369-377
28. Morgan C L et al., 1998, Immunogenetics 48/2): 98-107.
29. Nichols W G et al, 2000, J Clin Virol 16(1): 25-40
30. Nijman H W et al, 1993. Eur. J. Immunol., 6, 1215-9
31. Novak N et al., 2001. J. Immunol. 166: 6665-6670.
32. Parker, K C et al., 1994. J. Immunol. 152: 163.
33. Plotkin S A, 1999. Pediatr Infect Dis J 18(4): 313-25
34. Rammensee, H G et al., 1999. Immunogenetics 50: 213-219
35. Reddehase M J, 2000. Curr Opin Immunol. 12: 390-396
36. Retriere C et al, 2000. J Virol 74(9): 3948-3952
37. Saulquin X et al., 2000. Eur J Immunol 30 : 2531-2539
38. Sia I G et al, 2000. Clin Microbiol Rev 13(1): 83-121
39. Solache A et al., 1999. J. Immunol. 163: 5512-5518.
40. Stern L J et al., 1994. Structure 2: 245-251
41. Sturniolo T et al., 1999. Nature Biotechnology 17: 555-562.
42. Tana et al., 1998, J. Human Genet. 43(1): 14-21.
43. Tobery W T et al., 2001. J Immunol Methods 254: 59-66.
44. Valli A et al., 1993. J. Clin. Invest. 91: 616-628
45. Van den Eynde B J, et al., 1997. Curr Opin Immunol. 5: 684-93
46. van Son W J, 1999. Intervirology 42(5-6): 285-290
47. Villadangos J A et al., 2000. Immunity 12: 233-239
48. Waldrop S L et al., 1998. J. Immunol. 161: 5284.
49. Wilson D B et al., 1999. J. Immunol. 163: 6424-6434.
50. Weekes M P et al., 1999a. J Immunol 162: 7569-7577.
51. Weekes M P et al., 1999b, J Virol 73(3): 8140-8150.
52. Zaia J A et al, 2000. Cytomegalovirus Prevention and Treatment in 2000. Hematology (Am Soc Hematol Educ Program) 2000: 339-355

1-30. (canceled)

31. A method for isolating at least one MHC/HLA molecule ligand or at least one complex comprising such a ligand and a MHC/HLA molecule, comprising:
providing a pool of ligands, the pool comprising at least one ligand that binds to a MHC/HLA molecule and at least one ligand that does not bind to a MHC/HLA molecule;
contacting a MHC/HLA molecule with the pool of ligands, thereby allowing binding of a ligand that has a binding capacity for the MHC/HLA molecule to the MHC/HLA molecule and allowing formation of at least one complex comprising the ligand and the MHC/HLA molecule; and
detecting the complex.

32. The method of claim 31, further comprising separating the complex from ligands that are not bound to a MHC/HLA molecule.

33. The method of claim 31, further comprising isolating the ligand from the complex.

34. The method of claim 31, further comprising characterizing the ligand.

35. The method of claim 34, wherein the characterizing of the ligand comprises mass spectroscopy, polypeptide sequencing, a binding assay, or identification of ligands by determination of their retention factors by chromatography, a spectroscopic technique, nuclear magnetic resonance (NMR), circular dichroism (CD), electron spin resonance (ESR), or a combination thereof.

36. The method of claim 31, further defined as a method of isolating at least one T cell epitope that has a binding capacity to a MHC/HLA molecule and/or a complex comprising the epitope and the MHC/HLA molecule comprising:
separating the complex from ligands that do not bind to the MHC/HLA molecule; and
assaying the ligand and/or the complex in a T cell assay for T cell activation capacity.

37. The method of claim 36, further comprising characterizing the ligand.

38. The method of claim 36, further comprising isolating the ligand from the complex.

39. The method of claim 36, further comprising providing the ligand with a T cell activation capacity as T cell epitope or as complex to a subject.

40. The method of claim 36, wherein the T cell assay comprises the mixing and incubation of the complex with isolated T cells and subsequent measuring cytokine secretion or proliferation of the isolated T cells.

41. The method of claim 36, wherein the T cell assay comprises measuring up-regulation of activation markers or down-regulation of surface markers.

42. The method of claim 41, wherein the activation markers are CD69 or CD38.

43. The method of claim 41, wherein the surface markers are CD3, CD8 or TCR.

44. The method of claim 36, wherein the T cell assay comprises measuring up-/down-regulation of mRNAs involved in T cell activation.

45. The method of claim 44, wherein the assay is performed using real-time RT-PCR.

46. The method of claim 31, wherein the T cell assay is a T cell assay measuring phosphorylation/de-phosphorylation of components downstream of the T cell receptor, intracellular Ca++ concentration or activation of Ca++-dependent proteins, formation of immunological synapses, release of effector molecules, or a combination of T cell assays.

47. The method of claim 36, further defined as comprising identifying a T cell epitope.

48. The method of claim 47, wherein the identified T cell epitope comprises the sequence KMQVIGDQYV (SEQ ID NO:2), FTWPPWQAGI (SEQ ID NO:3), AMAGASTSA (SEQ ID NO:4), SDNEIHNPAV (SEQ ID NO:5), KYQEFFWDA (SEQ ID NO:6) or a combination thereof.

49. The method of claim 31, wherein the MHC/HLA molecule is a HLA class I molecule, HLA class II molecule, a non-classical MHC/HLA, a MHC/HLA-like molecule or a mixture thereof.

50. The method of claim 31, wherein the MHC/HLA molecule is a HLA A0201 molecule.

51. The method of claim 50, wherein the ligand is further defined as a polypeptide comprising the sequence RLLQTGIHV (SEQ ID NO:7), VIGDQYVKV (SEQ ID NO:8), YLESFCEDV (SEQ ID NO:9) or a combination thereof.

52. The method of claim 31, wherein the pool of ligands is a pool of peptides, a pool of protein fragments, a pool of glycolipids, a pool of glycosphingolipids, a pool of lipopeptides, a pool of lipids, a pool of glycans, a pool of modified peptides, a pool obtained from antigen-presenting cells, a pool comprised of fragments of cells, a pool comprised of peptide libraries, a pool of (poly)-peptides generated from recombinant DNA libraries, a pool of proteins and/or protein fragments from a specific pathogen or mixtures thereof.

53. The method of claim 31, further comprising a cytokine secretion assay, an intracellular cytokine staining, FACS or an ELISA.

54. The method of claim 53, wherein the cytokine secretion assay is an Elispot assay.

55. The method of claim 31, wherein the ligand is an antigen fragment.

56. The method of claim 55, wherein the antigen fragment is a fragment of a human immune deficiency virus (HIV), hepatitis A or B virus, hepatitis C virus (HCV), Rous sarcoma virus (RSV), Epstein Barr virus (EBV), Influenza virus, Rotavirus, Staphylococcus aureus, Chlamydia pneumoniae, Chlamydia trachomatis, Mycobacterium tuberculosis, Streptococcus pneumoniae, Bacillus antracis, Vibrio cholerae, Plasmodium sp., Aspergillus sp., Candida albicans, tumor, or autoimmune antigen.

57. The method of claim 31, wherein the ligand is a HCV-, HIV-, HAV-, HBV-, RSV-, EBV-, Influenza virus- or Rotavirus-peptide having a binding capacity for an MHC/HLA-molecule.

58. A method of isolating at least one T cell epitope that has a binding capacity to a MHC/HLA molecule and/or a complex comprising the epitope and the MHC/HLA molecule comprising:
providing a pool of ligands, the pool comprising at least one ligand that binds to a MHC/HLA molecule and at least one ligand that does not bind to a MHC/HLA molecule;
contacting a MHC/HLA molecule with the pool of ligands, thereby allowing binding of a ligand that has a binding capacity for the MHC/HLA molecule to the MHC/HLA molecule and allowing formation of at least one complex comprising the ligand and the MHC/HLA molecule;
separating the complex from ligands that do not bind to the MHC/HLA molecule; and
assaying the ligand and/or the complex in a T cell assay for T cell activation capacity.

59. A peptide and/or epitope isolated by the practice of the method of claim 31.

60. The peptide and/or epitope of claim 59, further defined as comprising the sequence KMQVIGDQYV (SEQ ID NO:2), FTWPPWQAGI (SEQ ID NO:3), AMAGASTSA (SEQ ID NO:4), SDNEIHNPAV (SEQ ID NO:5), KYQEFFWDA (SEQ ID NO:6), RLLQTGIHV (SEQ ID NO:7), VIGDQYVKV (SEQ ID NO:8), YLESFCEDV (SEQ ID NO:9), RPHERNGFTV (SEQ ID NO:10), TPRVTGGGAM (SEQ ID NO:12), DDVWTSGSDSDE (SEQ ID NO:11), a sequence of peptide No. 55-64, 109, 383, 384, 421, 449-454, 469, and/or 470 of table 3, or a combination thereof.

61. The peptide and/or epitope of claim 60, further defined as a T cell epitope comprising the sequence KMQVIGDQYV (SEQ ID NO:2), FTWPPWQAGI (SEQ ID NO:3), AMAGASTSA (SEQ ID NO:4), SDNEIHNPAV (SEQ ID NO:5), KYQEFFWDA (SEQ ID NO:6) or a combination thereof.

62. The peptide and/or epitope of claim 60, further defined as a HLA A0201 binding epitope with T cell activating capacity comprising the sequence RLLQTGIHV (SEQ ID NO:7), VIGDQYVKV (SEQ ID NO:8), YLESFCEDV (SEQ ID NO:9) or a combination thereof.

63. The peptide and/or epitope of claim 60, further defined as comprising the sequence of peptide No. 55-64, 109, 383, 384, 421, 449-454, 469 and/or 470 according to table 3.

64. The peptide and/or epitope of claim 59, further defined as comprising 1 to 30 naturally occurring amino acid residues.

65. The peptide and/or epitope of claim 64, further defined as comprising 2 to 10 naturally occurring amino acid residues.

66. The peptide and/or epitope of claim 65, further defined as comprising 2 to 6 naturally occurring amino acid residues.

67. The peptide and/or epitope of claim 64, wherein the naturally occurring amino acid residues are at the N-terminus, the C-terminus or at the N- and C-terminus of the peptide and/or epitope.

68. The peptide and/or epitope of claim 59, further defined as comprising a non-naturally occurring amino acid.

69. The peptide and/or epitope of claim 68, further defined as comprising 1 to 1000 non-naturally occurring amino acid residues.

70. The peptide and/or epitope of claim 69, further defined as comprising 2 to 100 non-naturally occurring amino acid residues.

71. The peptide and/or epitope of claim 70, further defined as comprising 2 to 20 non-naturally occurring amino acid residues.

72. The peptide and/or epitope of claim 68, wherein the non-naturally occurring amino acid residues are at the N-terminus, the C-terminus, or the N- and C-terminus of the peptide and/or epitope.

73. The peptide and/or epitope of claim 59, further defined as comprised in a vaccine.

74. A vaccine for treating or preventing cytomegalovirus (CMV) infections comprising a peptide and/or epitope of claim 59 in a pharmaceutically acceptable carrier.

75. The vaccine of claim 74, further defined as an HLA specific vaccine.

76. The vaccine of claim 74, further comprising an immunomodulating substance.

77. The vaccine of claim 76, wherein the immunomodulating substance is a polycationic substance, an immunomodulating nucleic acid, or a mixture thereof.

78. The vaccine of claim 77, wherein the immunomodulating substance is a polycationic polypeptide, an immunomodulating nucleic acid, or a mixture thereof.

79. The vaccine of claim 77, wherein the immunomodulating substance is a deoxyinosine and/or deoxyuracile containing oligodeoxynucleotide.

80. A method of vaccinating a subject to treat or prevent a cytomegalovirus (CMV) infection comprising providing to the subject a peptide and/or epitope of claim 59.

81. The method of clam 79, wherein the peptide or epitope is provided via administration of a peptide, peptide analogue, protein, naked DNA, RNA, viral vector, virus-like particle, recombinant/chimeric virus, recombinant bacteria, or dendritic cell that has been pulsed with protein/peptide/RNA or transfected with DNA encoding the peptide or epitope.

82. A T cell, a T cell clone, a T cell population, or T cell preparation that specifically recognizes a peptide and/or epitope of claim 59.

83. A method of identifying heteroclitic epitopes comprising using a T cell, a T cell clone, a T cell population, or T cell preparation that specifically recognizes a peptide and/or epitope of claim 59.

84. A method of preparing c composition for CMV therapy comprising using a T cell, a T cell clone, a T cell population, or T cell preparation that specifically recognizes a peptide and/or epitope of claim 59.

85. A method of activating T cells, comprising administering to the individual a peptide comprising the sequence RPHERNGFTV (SEQ ID NO:10), TPRVTGGGAM (SEQ ID NO:12), DDVWTSGSDSDE (SEQ ID NO:11), or a combination thereof.

86. The method of claim 85, further defined as a method of activating T cells in a B7-negative individual comprising administering to the individual a peptide comprising the sequence RPHERNGFTV (SEQ ID NO:10).

87. The method of claim 85, further defined as a method of activating T cells in a B7-negative individual comprising administering to the individual a peptide comprising the sequence TPRVTGGGAM (SEQ ID NO:12).

88. The method of claim 85, further defined as a method of activating T cells in a B35-negative individual comprising administering to the individual a peptide comprising the sequence DDVWTSGSDSDE (SEQ ID NO:11).
